(19) United States
(12) Patent Application Publication
(10) Pub. No.: US 2018/0044733 A1
(43) Pub. Date: Feb. 15, 2018

(54) Circulatory MicroRNAs (miRNAs) as Biomarkers for Diabetic Retinopathy (DR) and Age-Related Macular Degeneration

(71) Applicant: The Regents of the University of California, Oakland, CA (US)

(72) Inventors: Zeljka Smit-McBride, Davis, CA (US); Lawrence Morse, Davis, CA (US)

(21) Appl. No.: 15/308,559

(22) PCT Filed: May 4, 2015
(86) PCT No.: PCT/US15/29080

§ 371 Date: Nov. 2, 2016

Related U.S. Application Data
(60) Provisional application No. 61/988,764, filed on May 5, 2014.

(51) Int. Cl. C12Q 1/68

(52) U.S. Cl. CPC C12Q 1/6883; C12Q 2600/106; C12Q 2600/178

(57) ABSTRACT

The present disclosure provides methods that find use in determining a likelihood of having or developing diabetic retinopathy (DR) in a subject. Such methods generally include detection of one or more DR diagnostic microRNAs (miRNAs) as described herein. The detection of one or more (e.g., a combination) of such DR diagnostic miRNAs can be used to determine a likelihood of having or developing DR in a subject. Also provided are wet and dry age-related macular degeneration (AMD) diagnostic miRNAs and methods of using same.

### CROSS-REFERENCE

This invention claims the benefit of U.S. Provisional Application No. 61/988,764, filed May 5, 2014, which application is hereby incorporated by reference in its entirety herein.

### STATEMENT REGARDING FEDERALLY SPONSORED RESEARCH

This invention was made with government support under Grant Number R01EY021537 awarded by the National Institutes of Health. The government has certain rights in the invention.

### INTRODUCTION

Diabetic retinopathy (DR), diabetic macular edema and associated conditions, are the leading and growing causes of vision impairment and blindness in the United States and throughout the world. There is no cure and the current anti-angiogenic therapies, though effective at reducing vessel growth and permeability, do not address the underlying pathogenesis. Currently, only invasive treatments are available for these disorders—surgery and intravitreal injection of anti-inflammatory agents. Both alleviate issues only temporarily, and vision loss is frequently unavoidable. Thus, the need for new scientific and therapeutic approaches is clear and compelling.

Macular degeneration, or age-related macular degeneration (AMD), is a leading cause of vision loss among people age 50 and older. It causes damage to the macula, a small region near the center of the retina and the part of the eye needed for sharp, central vision. There are two types: wet and dry. Wet AMD occurs when abnormal blood vessels grow under the macula. These new blood vessels often leak blood and fluid. Wet AMD damages the macula quickly. Blurred vision is a common early symptom. Dry AMD occurs when the light-sensitive cells in the macula slowly break down, resulting in a gradual loss of central vision.

Recently, a novel type of RNA, microRNA (miRNAs), has been implicated in many human diseases. MiRNAs are a class of small non-coding RNAs that regulate gene expression at the posttranscriptional level by either degrading or blocking translation of messenger RNA targets. Besides their presence in tissues, miRNAs circulate in the bloodstream in a highly stable, extracellular form and are being developed as blood-based biomarkers for cancer and many other diseases.

Circulatory microRNAs have been shown to be differentially expressed in serum and plasma studies in diabetics, urine, retina and retinal endothelial cells (RECs) of streptozocin (STZ)-induced diabetic rats.

### SUMMARY

The present disclosure provides methods that find use in determining a likelihood of having or developing diabetic retinopathy (DR) in a subject. Such methods generally include detection of one or more DR diagnostic microRNAs (miRNAs) as described herein. The detection of one or more (e.g., a combination) of such DR diagnostic miRNAs can be used to determine a likelihood of having or developing DR in a subject.

The present disclosure also provides methods that find use in determining a likelihood of having or developing dry or wet age-related macular degeneration (AMD) in a subject. Such methods generally include detection of one or more dry or wet AMD diagnostic miRNAs as described herein. The detection of one or more (e.g., a combination) of such dry or wet AMD diagnostic miRNAs can be used to determine a likelihood of having or developing dry or wet AMD in a subject. In some embodiments, the present disclosure provides methods that find use in determining a likelihood of having or developing a resistance to anti-VEGF therapy in a subject suffering from age related macular degeneration (AMD).

The methods of the present disclosure also find use in facilitating treatment decisions for a subject. Also provided are devices, systems, and kits that may be used in practicing methods of the present disclosure.

In some embodiments, methods of determining a likelihood of having or developing DR in a subject are provided, wherein the method includes: determining an amount of at least one DR diagnostic miRNA in a biological sample from the subject; comparing the amount of the at least one DR diagnostic miRNA with a control amount of the at least one DR diagnostic miRNA; and generating a report indicating a likelihood of having or developing DR in the subject based on results of said comparing the amount of the at least one DR diagnostic miRNA with the control amount of the at least one DR diagnostic miRNA.

In some embodiments, methods of determining efficacy of a therapy for treatment of DR in a subject are provided, wherein the method includes: determining an amount of at least one DR diagnostic miRNA in a biological sample from the subject prior to administration of the therapy; administering the therapy; determining an amount of the least one DR diagnostic miRNA in a biological sample from the subject following administration of the therapy; comparing the amount of the at least one DR diagnostic miRNA in a biological sample prior to administration of the therapy with the amount of the at least one DR diagnostic miRNA in a biological sample following administration of the therapy; and generating a report indicating efficacy of the therapy based on results of said comparing.

In some embodiments, methods of determining a likelihood of having or developing dry or wet AMD in a subject are provided, wherein the method includes: determining an amount of at least one dry or wet AMD diagnostic miRNA in a biological sample from the subject; comparing the amount of the at least one dry or wet AMD diagnostic miRNA with a control amount of the at least one dry or wet AMD diagnostic miRNA; and generating a report indicating a likelihood of having or developing dry or wet AMD in the subject based on results of said comparing the amount of the at least one dry or wet AMD diagnostic miRNA with the control amount of the at least one dry or wet AMD diagnostic miRNA.

In some embodiments, methods of determining efficacy of a therapy for treatment of dry or wet AMD in a subject are provided, wherein the method includes: determining an amount of at least one dry or wet AMD diagnostic miRNA in a biological sample from the subject prior to administration of the therapy; administering the therapy; determining an amount of the least one dry or wet AMD diagnostic miRNA in a biological sample from the subject following administration of the therapy; comparing the amount of the at least one dry or wet AMD diagnostic miRNA in a biological sample prior to administration of the therapy with the amount of the at least one dry or wet AMD diagnostic miRNA in a biological sample following administration of the therapy; and generating a report indicating efficacy of the therapy based on results of said comparing.

In some embodiments, methods of determining a likelihood of having or developing a resistance to anti-VEGF therapy in a subject suffering from AMD are provided, wherein the method includes: determining an amount of at least one anti-VEGF therapy resistance diagnostic miRNA in a biological sample from the subject; comparing the amount of the at least one anti-VEGF therapy resistance diagnostic miRNA with a control amount of the at least one anti-VEGF therapy resistance diagnostic miRNA; and generating a report indicating a likelihood of having or developing anti-VEGF therapy resistance in the subject based on results of said comparing the amount of the at least one anti-VEGF therapy resistance diagnostic miRNA with the control amount of the at least one anti-VEGF therapy resistance diagnostic miRNA.

These and other features will be apparent to the ordinarily skilled artisan upon reviewing the present disclosure.

### BRIEF DESCRIPTION OF THE DRAWINGS

The invention may be best understood from the following detailed description when read in conjunction with the accompanying drawings. Included in the drawings are the following figures:

FIG. 1 shows bioanalyzer profiles of small RNA populations in ocular fluids. Top panel represent aqueous, and bottom vitreous. Each of the compartments showed the presence of microRNA (10-40 nt), and an abundance of small RNA in a larger size range (>40 nt).

FIG. 2 shows the relative amount of miRNAs found in aqueous and vitreous in control samples.

FIG. 3 shows the fold change in abundance of miRNAs miR-let-7a, miR-let-7b, miR-let-7c and miR-let-7d in particular diabetic groups compared to the control group.

FIG. 4 shows the fold change in abundance of miR-320a, miR-320b, miR-320c and mir-320e in particular diabetic groups compared to the control group.

FIG. 5 shows the Top Gene Network affected by ubiquitously expressed miRNA in aqueous and vitreous.

FIG. 6 shows a partial interactome of let-7 family of miRNAs.

FIG. 7 shows a partial interactome of miR-320 family of miRNAs.

FIG. 8 shows a table listing the source of aqueous samples used in the analysis. Abbreviations: RD retinal detachment; MH macular hole; ERM(S) epiretinal membranes; (N)IDDM (non-) insulin-dependent diabetes mellitus; CON normal; DR-Typ I/II diabetic retinopathy, Type I/II diabetes mellitus; DNOR no retinopathy, Type II diabetes; OD oculus dexter; OU oculus uterque; OS oculus sinister; PVR Posterior vitreous detachment; VH vitreous hemorrhage; TRD tractional retinal detachment; VMT vitreo-macular traction.

FIG. 9 shows a table listing the source of aqueous samples used in the analysis.

FIG. 10 shows another bioanalyzer profile of small RNA populations in ocular fluids.

FIG. 11 shows a heat map of miRNA expression patterns based on microarray miRNA analysis of serum samples derived from the indicated patient groups. Data points were grouped according to disease states.

FIG. 12 shows microarray expression levels of serum miRNAs that showed a statistically significant difference between control and wet AMD groups. Values are expressed in fluorescence intensity compared to a reference. All miRNAs listed showed statistical significance (p<0.01).

FIG. 13 shows microarray expression levels of serum miRNAs that showed a statistically significant difference between control and dry AMD groups. Values are expressed in fluorescence intensity compared to a reference. All miRNAs listed showed statistical significance (p<0.01).

FIG. 14 shows microarray expression levels of serum miRNAs that showed a statistically significant difference between control and resistant AMD groups. Values are expressed in fluorescence intensity compared to a reference. All miRNAs listed showed statistical significance (p<0.01).

FIG. 15 shows microarray expression levels of serum miRNA that are highly expressed, compared among the different control and patient groups. For each miRNA listed on the x-axis, the grouped bars from left to right are CON Ave, WET Ave, RES Ave, and DRY Ave.

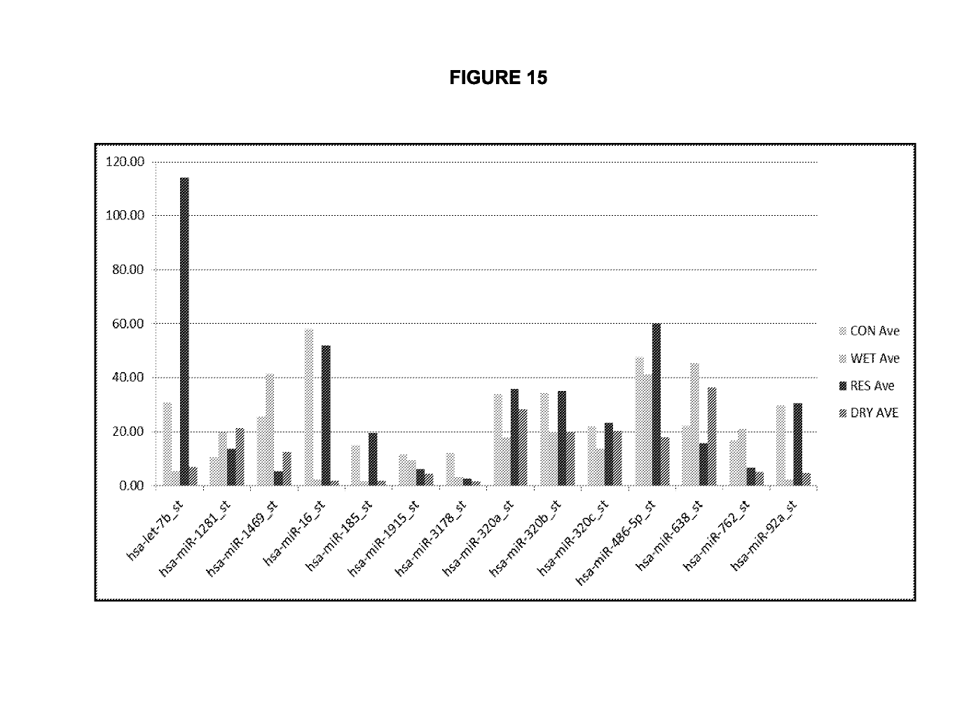

FIG. 16 shows qPCR expression levels of miR-486 in serum samples from control, dry AMD and wet AMD groups (n=8). Data points are expressed as fold change compared to controls. Values were normalized to three reference genes (miR-103a, miR-191, and miR-423) found in serum.

FIG. 17 shows qPCR expression levels of miR-let-7b in serum samples from control, wet AMD and resistant wet AMD groups (n=8).

FIG. 18 provides a graph showing fold change in expression of select let-7 miRNA family members in aqueous humor of patients [Diabetes Mellitus, Type 1 (DMI); Diabetes Mellitus, Type II, with proliferative retinopathy (DMII-PDR); Diabetes Mellitus, Type II, with non-proliferative retinopathy (DMII-NPDR); and Diabetes Mellitus, Type II, with no retinopathy (DMII-noDR)].

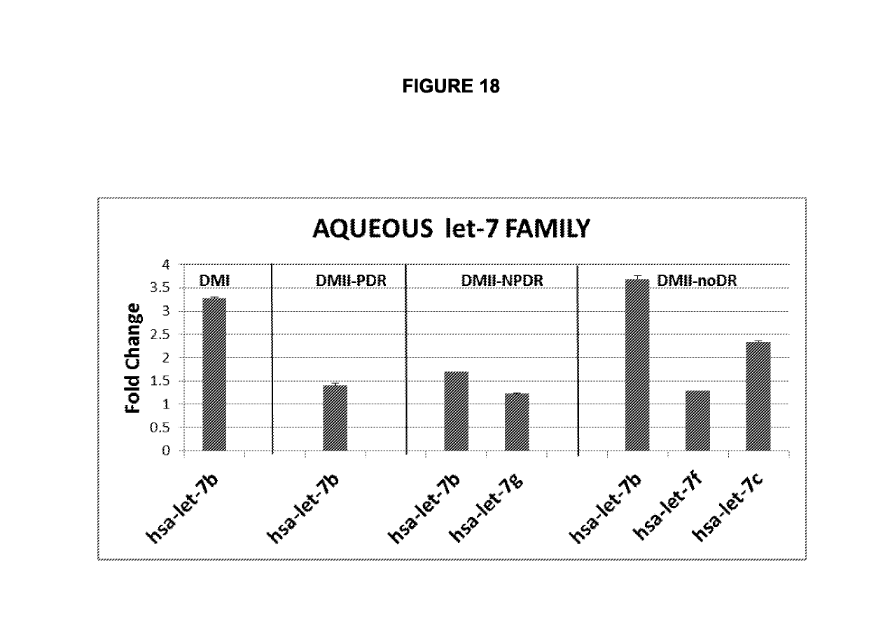

FIG. 19 provides a graph showing fold change in expression of select let-7 miRNA family members in vitreous humor of patients [Diabetes Mellitus, Type 1 (DMI); Diabetes Mellitus, Type II, with proliferative retinopathy (DMII-PDR); Diabetes Mellitus, Type II, with non-proliferative retinopathy (DMII-NPDR); and Diabetes Mellitus, Type II, with no retinopathy (DMII-noDR)].

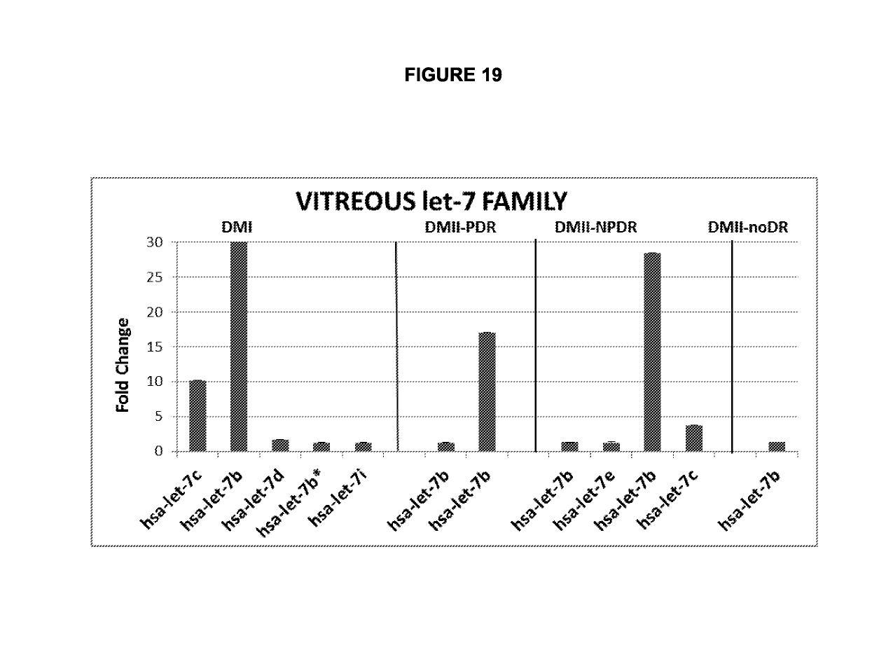

FIG. 20 provides a graph showing fold change in expression of select miR-320 family members in aqueous humor of patients [Diabetes Mellitus, Type II, with proliferative retinopathy (DMII-PDR); Diabetes Mellitus, Type II, with non-proliferative retinopathy (DMII-NPDR); and Diabetes Mellitus, Type II, with no retinopathy (DMII-noDR)].

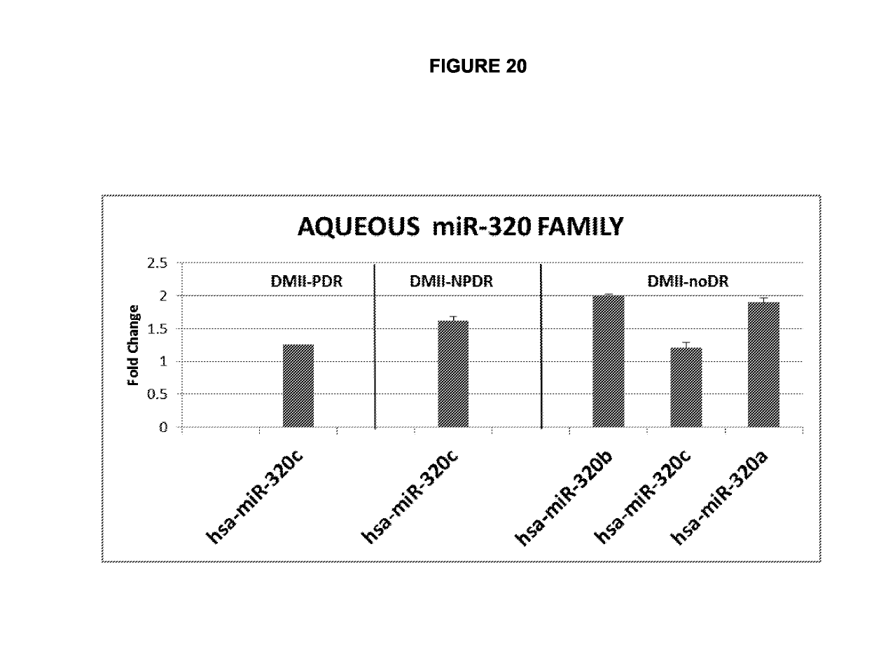

FIG. 21 provides a graph showing fold change in expression of select miR-320 family members in vitreous humor of patients [Diabetes Mellitus, Type 1 (DMI); Diabetes Mellitus, Type II, with proliferative retinopathy (DMII-PDR); and Diabetes Mellitus, Type II, with no retinopathy (DMII-noDR)].

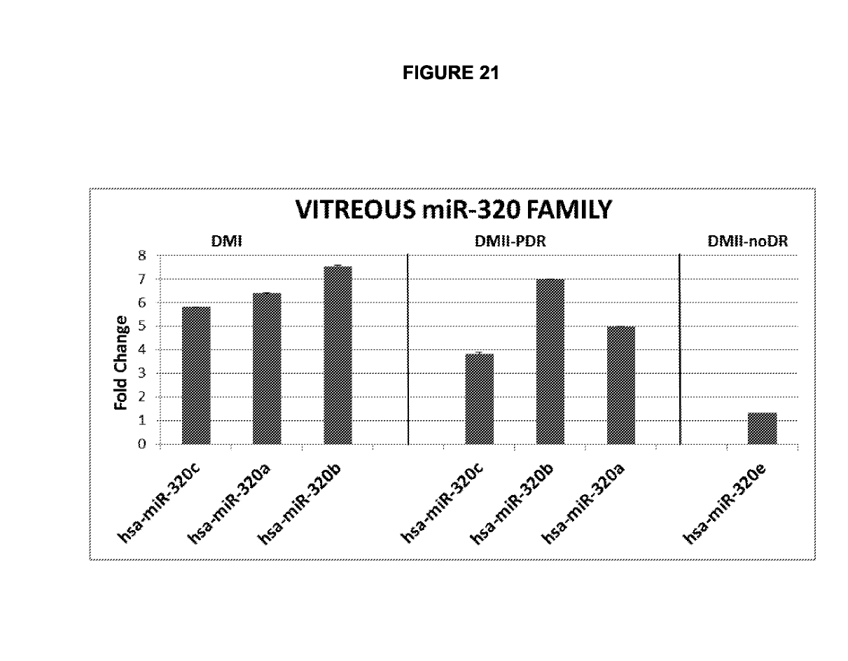

FIG. 22 provides a graph showing fold change in expression of select let-7 miRNA family members in plasma of patients [Diabetes Mellitus, Type 1 (DMI); Diabetes Mellitus, Type II, with proliferative retinopathy (DMII-PDR); and Diabetes Mellitus, Type II, with no retinopathy (DMII-noDR)].

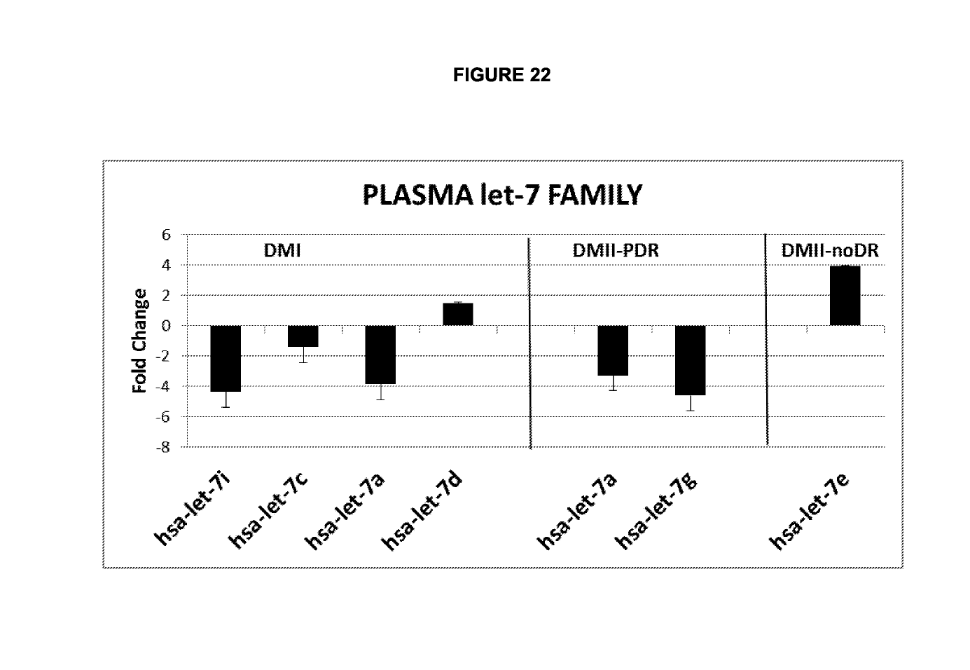

FIG. 23 provides a graph showing fold change in expression of select miR-320 family members in plasma of patients [Diabetes Mellitus, Type 1 (DMI); Diabetes Mellitus, Type II, with non-proliferative retinopathy (DMII-NPDR); and Diabetes Mellitus, Type II, with no retinopathy (DMII-noPDR)].

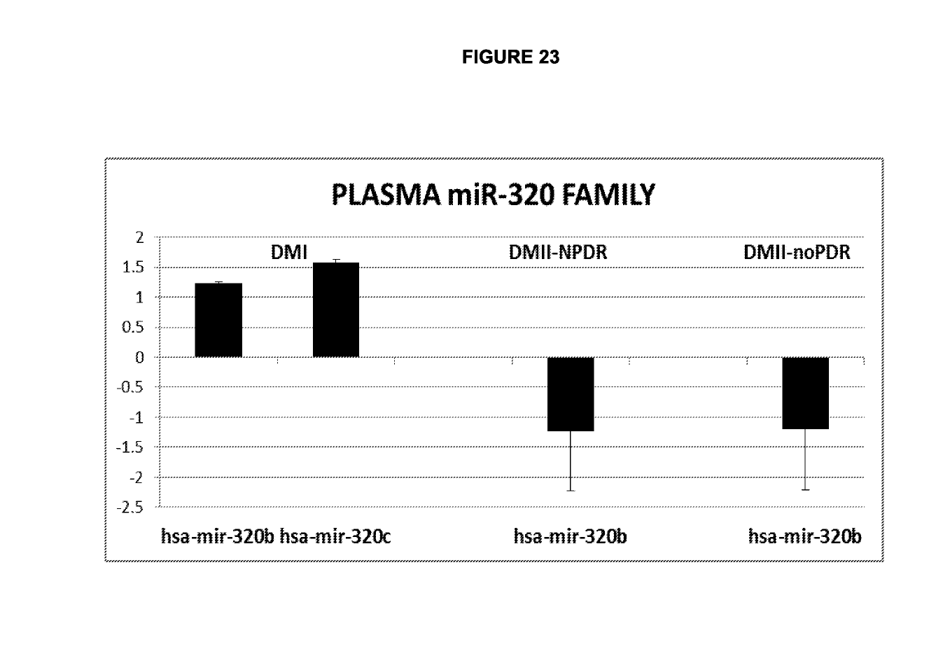

FIG. 24 provides a graph showing fold change in expression for select miRNAs in patients with Type I diabetes with retinopathy compared to control.

FIG. 25 provides a graph showing fold change in expression for select miRNAs in patients with Type II diabetes with retinopathy compared to control.

FIG. 26 provides a graph showing fold change in expression for select miRNAs in patients with Type II diabetes without retinopathy compared to control.

FIG. 27 provides a graph showing fold change in expression for select miRNAs in patients with Type I diabetes with retinopathy, Type II diabetes with retinopathy, and Type I diabetes without retinopathy compared to control.

FIG. 28 provides a graph showing expression of select let-7 miRNA family members in aqueous humor of various patient groups as determined by qPCR.

FIG. 29 provides a graph showing expression of select let-7 miRNA family members in vitreous humor of various patient groups as determined by qPCR.

FIG. 30 provides a graph showing expression of select let-7 miRNA family members in plasma of various patient groups as determined by qPCR.

FIG. 31 provides a graph showing expression of select miR-320 family members in aqueous humor of various patient groups as determined by qPCR.

FIG. 32 provides a graph showing expression of select miR-320 family members in vitreous humor of various patient groups as determined by qPCR.

FIG. 33 provides a graph showing expression of select miR-320 family members in serum of various patient groups as determined by qPCR.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a biological sample” includes a plurality of such biological samples and reference to “the processor” includes reference to one or more processors, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

A “biomarker” or “marker” as used herein generally refers to an organic biomolecule (e.g., a microRNA) which is differentially present in a sample taken from a subject of one phenotypic status (e.g., having a disease) as compared with another phenotypic status (e.g., not having the disease or having a different disease). A biomarker is differentially present between different phenotypic statuses if the mean or median level of the biomarker in a first phenotypic status relative to a second phenotypic status is calculated to represent statistically significant differences. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. As such, biomarkers can find use as markers for, for example, disease (diagnostics), therapeutic effectiveness of a drug (theranostics), and the like. Biomarkers are thus analytes in assays that facilitate diagnosis, theranostics, and the like.

The terms “microRNA” and “miRNA” are used in accord with their ordinary usage in the art. Generally speaking, miRNA are short (e.g., about 18-24 nucleotides in length), non-coding RNAs, which regulate gene expression post-transcriptionally by destabilizing messenger RNAs (mRNA) and/or inhibiting their translation. Canonical miRNAs derive from longer polymerase II transcripts, called pri-miRNAs. A complex consisting of the proteins DGCR8 and Drosha process the pri-miRNAs to pre-miRNAs, which are then exported to the cytoplasm and cleaved by the protein Dicer to mature miRNAs. Exceptions to this processing include non-canonical miRNAs that bypass DGCR8/Drosha, while still being processed by Dicer. Knockout models of Dgcr8 and Dicer have been developed that remove only canonical miRNAs or both canonical and non-canonical miRNAs, respectively.

The term “diabetic retinopathy (DR) diagnostic miRNA” or “DR diagnostic miRNA” refers to one or more of the miRNAs identified herein (or a precursor or derivative thereof) as being differentially present in a sample taken from a subject having DR as compared with a subject not having DR. A DR diagnostic miRNA is a biomarker.

The term “dry or wet age-related macular degeneration (AMD) diagnostic miRNA” or “dry or wet AMD diagnostic miRNA” refers to one or more of the miRNAs identified herein (or a precursor or derivative thereof) as being differentially present in a sample taken from a subject having AMD as compared with a subject not having AMD. An AMD diagnostic miRNA is a biomarker.

The term “anti-Vascular Endothelial Growth Factor (VEGF) therapy resistance diagnostic miRNA” or “anti-VEGF therapy resistance diagnostic miRNA” refers to one or more of the miRNAs identified herein (or a precursor or derivative thereof) as being differentially present in a sample taken from a subject resistant to VEGF therapy as compared with a subject not resistant to VEGF therapy. An anti-VEGF therapy resistance diagnostic miRNA is a biomarker.

The terms “individual,” “subject,” and “patient,” used interchangeably herein, refer to mammal, e.g., a human.

The term “healthy individual” in the context of the methods of the present disclosure refers to an individual who is unaffected by a detectable illness, particularly DR or AMD.

A “biological sample” encompasses a variety of sample types obtained from an individual. The definition encompasses biological fluids (e.g., blood (including blood fractions (e.g., serum, plasma)); and other liquid samples of biological origin (e.g., saliva, urine, bile fluid, vitreous humor, and aqueous humor), as well as solid tissue samples. “Blood sample” refers to a biological sample, which is obtained from blood of a subject, and includes whole blood and blood fractions (e.g., plasma or serum) suitable for analysis in the present methods. In general, separation of cellular components and non-cellular components in a blood sample (e.g., by centrifugation) without coagulation provides a blood plasma sample, while such separation of coagulated (clotted) blood provides a blood serum sample. Examples of biological samples of blood include peripheral blood or samples derived from peripheral blood. The definition also includes samples that have been manipulated after their procurement, such as by treatment with reagents, solubilization, or enrichment for certain components, such as one or more polypeptides to be assayed. For example, a biological sample (e.g., blood) can be enriched for a fraction containing an analyte(s) of interest.

“Isolated” refers to an entity of interest that is in an environment different from that in which the compound may naturally occur. “Isolated” is meant to include compounds that are within samples that are substantially enriched for the compound of interest and/or in which the compound of interest is partially or substantially purified.

By “purified” is meant a compound of interest (e.g., an RNA, DNA or polypeptide) has been separated from components that accompany it in nature. “Purified” can also be used to refer to a compound of interest separated from components that can accompany it during manufacture (e.g., in chemical synthesis). In some embodiments, a compound is substantially pure when it is at least 50% to 60%, by weight, free from organic molecules with which it is naturally associated or with which it is associated during manufacture. In some embodiments, the preparation is at least 75%, at least 90%, at least 95%, or at least 99%, by weight, of the compound of interest. A substantially pure compound can be obtained, for example, by extraction from a natural source (e.g., bacteria), by chemically synthesizing a compound, or by a combination of purification and chemical modification. A substantially pure compound can also be obtained by, for example, enriching a sample that contains the compound. A substantially pure compound can also be obtained by recombinant or chemical synthetic production. Purity can be measured by any appropriate method, e.g., chromatography, mass spectroscopy, high performance liquid chromatography analysis, etc.

As used herein, the terms “determining”, “assessing”, “assaying”, “measuring” and “detecting” refer to both quantitative and semi-quantitative determinations and as such, the term “determining” is used interchangeably herein with “assaying,” “measuring,” and the like. Where a quantitative determination is intended, the phrase “determining an amount” of an analyte and the like is used. Where either a quantitative and semi-quantitative determination is intended, the phrase “determining a level” of an analyte or “detecting” an analyte is used.

“Quantitative” assays in general provide information on the amount of an analyte in a sample relative to a reference (control), and are usually reported numerically, where a “zero” value can be assigned where the analyte is below the limit of detection. “Semi-quantitative” assays involve presentation of a numeric representation of the amount of the analyte in the specimen that is relative to a reference (e.g., a threshold, e.g., normal threshold or an abnormal threshold), where a “zero” value can be assigned where the analyte is below the limit of detection. In general, semi-quantitative results are compared against an accompanying reference interval to provide a qualitative interpretation of the result.

“Sensitivity” refers to the fraction of people with the disease or disease risk level (e.g., diabetic retinopathy) that a test correctly identifies as positive. “Specificity” refers to the fraction of people without the disease or disease risk level that the test correctly identifies as negative. The fractions with respect to sensitivity and/or specificity may be presented as a percentage. Where expressed as percentages, specificity can be calculated as by subtracting the sensitivity value for incorrect diagnosis from 100.

The term “primer” may refer to more than one primer and refers to an oligonucleotide, whether occurring naturally, or produced synthetically, which is capable of acting as a point of initiation of synthesis along a complementary strand when placed under conditions in which synthesis of a primer extension product which is complementary to a nucleic acid strand is catalyzed. Such conditions include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.), and at a suitable temperature. The primer is preferably single-stranded for maximum efficiency in amplification.

The complement of a nucleic acid sequence as used herein refers to an oligonucleotide which, when aligned with the nucleic acid sequence such that the 5′ end of one sequence is paired with the 3′ end of the other, is in “antiparallel association.” Complementarity need not be perfect; stable duplexes may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

The terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

### Methods of Determining a Likelihood of Having or Developing Diabetic Retinopathy (DR)

As summarized above, aspects of the present disclosure include methods for determining a likelihood of having or developing diabetic retinopathy (DR) in a subject. Such a determination of the likelihood of having or developing DR may include computing a likelihood of having or developing DR in the subject so as to differentiate the subject from a healthy individual. A determination of the likelihood of having or developing DR can involve differentiating the subject from an individual having DR.

In general, the methods involve determining an amount of at least one DR diagnostic miRNA in a biological sample from the subject; comparing the amount of the at least one DR diagnostic miRNA with a control amount of the at least one DR diagnostic miRNA; and generating a report indicating a likelihood of having or developing DR in the subject based on results of said comparing the amount of the at least one DR diagnostic miRNA with the control amount of the at least one DR diagnostic miRNA. The detection of one or more such DR diagnostic miRNAs can be used to determine a likelihood of having or developing DR in a subject. The methods of the present disclosure also find use in facilitating treatment decisions for a subject.

The DR diagnostic miRNAs used in the methods of the present disclosure, as well as the methods of detection and analysis, are described in more detail below.

DR Diagnostic miRNAs for Detection

In some embodiments, the methods of present disclosure involve detection of a DR diagnostic miRNA in a biological sample of a patient. Specifically, in some embodiments, the present methods involve detection of one or more of the miRNAs identified herein (or a precursor or derivative thereof) as being differentially present in a sample taken from a subject having DR as compared with a subject not having DR. Such miRNAs include, e.g., miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548.

In certain embodiments, the methods involve detection of or determination of an amount of one of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. In certain embodiments, the methods involve detection of or determination of an amount of any two of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. In certain embodiments, the methods involve detection of or determination of an amount of any three of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. In certain embodiments, the methods involve detection of or determination of an amount of any four of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. In certain embodiments, the methods involve detection of or determination of an amount of any five of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. In certain embodiments, the methods involve detection of or determination of an amount of any six of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. In certain embodiments, the methods involve detection of or determination of an amount of any seven of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. In certain embodiments, the methods involve detection of or determination of an amount of any eight of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. In certain embodiments, the methods involve detection of or determination of an amount of any nine of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. In certain embodiments, the methods involve detection of or determination of an amount of any ten of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. In certain embodiments, the methods involve detection of or determination of an amount of any eleven of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. In certain embodiments, the methods involve detection of or determination of an amount of any twelve of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. In certain embodiments, the methods involve detection of or determination of an amount of any thirteen of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. In certain embodiments, the methods involve detection of or determination of an amount of all of miR-let-7a, miR-let-7b, miR-let-7c, miR-let-7d, miR-320a, miR-320b, miR-320c, miR-320e, miR-26a, miR-4488, miR-638, miR-29a, miR-4662a, and miR-548. The methods can further involve detection of other biomarkers.

miR-let-7a

The DR diagnostic miRNA miR-let-7a, which may also be referred to as “hsa-let-7a” is a member of the let-7 gene family. Examples of the stem-loop sequence for hsa-let-7a include those comprising a nucleic acid sequence of miRBase Accession No. MI0000060, MI0000061, MI0000062, or naturally occurring variants thereof. For example, the nucleic acid stem-loop sequence of MI0000060 is as follows: UGGGAUGAGGUAGUAGGUUGUAUAGUUUUAGGGUCACACCCACCACUG GGAGAUAACUAUACAAUCUACUGUCUUUCCUA; the nucleic acid stem-loop sequence of MI0000061 is as follows: AGGUUGAGGUAGUAGGUUGUAUAGUUUAGAAUUACAUCAAGGGAGAUA ACUGUACAGCCUCCUAGCUUUCCU; and the nucleic acid stem-loop sequence of MI0000062 is as follows:

GGGUGAGGUAGUAGGUUGUAUAGUUUGGGGCUCUGCCCUGCUAUGGGA
UAACUAUACAAUCUACUGUCUUUCCU.

The stem-loop sequence may be processed to the mature sequence of miR-let-7a. Examples of the mature sequence include those comprising a nucleic acid sequence of miRBase Accession No. MIMAT0000062, MIMAT0004481, or MIMAT0010195. For example, the nucleic acid sequence of MIMAT0000062 is as follows: UGAGGUAGUAGGUUGUAUAGUU; the nucleic acid sequence of MIMAT0004481 is as follows: CUAUACAAUCUACUGUCUUUC; and the nucleic acid sequence of MIMAT0010195 is as follows:

CUGUACAGCCUCCUAGCUUUCC.

Detection of miR-let-7a encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-let-7a can involve detection using one or more probes and/or primers.

miR-let-7b

The DR diagnostic miRNA miR-let-7b, which may also be referred to as “hsa-let-7b” is a member of the let-7 gene family. Examples of the stem-loop sequence for hsa-let-7b include those comprising a nucleic acid sequence of miRBase Accession No. MI0000063 or naturally occurring variants thereof. For example, the nucleic acid stem-loop sequence of MI0000063 is as follows:

CGGGGUGAGGUAGUAGGUUGUGUGGUUUCAGGGCAGUGAUGUUGCCCC
UCGGAAGAUAACUAUACAACCUACUGCCUUCCCUG.

The stem-loop sequence may be processed to the mature sequence of miR-let-7b. Examples of the mature sequence include those comprising a nucleic acid sequence of miRBase Accession No. MIMAT0000063 or MIMAT0004482. For example, the nucleic acid sequence of MIMAT0000063 is as follows: UGAGGUAGUAGGUUGUGUGGUU; and the nucleic acid sequence of MIMAT0004482 is as follows: CUAUACAACCUACUGCCUUCCC.

Detection of miR-let-7b encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-let-7b can involve detection using one or more probes and/or primers.

miR-let-7c

The DR diagnostic miRNA miR-let-7c, which may also be referred to as “hsa-let-7c” is a member of the let-7 gene family. Examples of the stem-loop sequence for hsa-let-7c include those comprising a nucleic acid sequence of miRBase Accession No. MI0000064 or naturally occurring variants thereof. For example, the nucleic acid stem-loop sequence of MI0000064 is as follows:

GCAUCCGGGUUGAGGUAGUAGGUUGUAUGGUUUAGAGUUACACCCUGG
GAGUUAACUGUACAACCUUCUAGCUUUCCUUGGAGC.

The stem-loop sequence may be processed to the mature sequence of miR-let-7c. Examples of the mature sequence include those comprising a nucleic acid sequence of miRBase Accession No. MIMAT0000064 or MIMAT0026472. For example, the nucleic acid sequence of MIMAT0000064 is as follows: UGAGGUAGUAGGUUGUAUGGUU; and the nucleic acid sequence of IMAT0026472 is as follows: CUGUACAACCUUCUAGCUUUCC.

Detection of miR-let-7c encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-let-7c can involve detection using one or more probes and/or primers.

miR-let-7d

The DR diagnostic miRNA miR-let-7d, which may also be referred to as “hsa-let-7d” is a member of the let-7 gene family. Examples of the stem-loop sequence for hsa-let-7d include those comprising a nucleic acid sequence of miRBase Accession No. MI0000065 or naturally occurring variants thereof. For example, the nucleic acid stem-loop sequence of MI0000065 is as follows:

CCUAGGAAGAGGUAGUAGGUUGCAUAGUUUUAGGGCAGGGAUUUUGCC
CACAAGGAGGUAACUAUACGACCUGCUGCCUUUCUUAGG.

The stem-loop sequence may be processed to the mature sequence of miR-let-7d. Examples of the mature sequence include those comprising a nucleic acid sequence of miRBase Accession No. MIMAT0000065 or MIMAT0004484. For example, the nucleic acid sequence of MIMAT0000065 is as follows: AGAGGUAGUAGGUUGCAUAGUU; and the nucleic acid sequence of MIMAT0004484 is as follows: CUAUACGACCUGCUGCCUUUCU.

Detection of miR-let-7d encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-let-7d can involve detection using one or more probes and/or primers.

miR-320a

The DR diagnostic miRNA miR-320a, which may also be referred to as “hsa-mir-320a” is a member of the miR-320 gene family. Examples of the stem-loop sequence for hsa-mir-320a include those comprising a nucleic acid sequence of miRBase Accession No. MI0000542 or naturally occurring variants thereof. For example, the nucleic acid stem-loop sequence of MI0000542 is as follows:

GCUUCGCUCCCCUCCGCCUUCUCUUCCCGGUUCUUCCCGGAGUCGGGAA
AAGCUGGGUUGAGAGGGCGAAAAAGGAUGAGGU.

The stem-loop sequence may be processed to the mature sequence of miR-320a. Examples of the mature sequence include those comprising a nucleic acid sequence of miRBase Accession No. MIMAT0000510. For example, the nucleic acid sequence of MIMAT0000510 is as follows: AAAAGCUGGGUUGAGAGGGCGA.

Detection of miR-320a encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-320a can involve detection using one or more probes and/or primers.

miR-320b

The DR diagnostic miRNA miR-320b, which may also be referred to as “hsa-mir-320b” is a member of the miR-320 gene family. Examples of the stem-loop sequence for hsa-mir-320b include those comprising a nucleic acid sequence of miRBase Accession No. MI0003776, MI0003839, or naturally occurring variants thereof. For example, the nucleic acid stem-loop sequence of MI0003776 is as follows: AAUUAAUCCCUCUCUUUCUAGUUCUUCCUAGAGUGAGGAAAAGCUGGG UUGAGAGGGCAAACAAAUUAACUAAUUAAUU; and the nucleic acid stem-loop sequence of MI0003839 is as follows:

UGUUAUUUUUUGUCUUCUACCUAAGAAUUCUGUCUCUUAGGCUUUCUC
UUCCCAGAUUUCCCAAAGUUGGGAAAAGCUGGGUUGAGAGGGCAAAAG
GAAAAAAAAAGAAUUCUGUCUCUGACAUAAUUAGAUAGGGA.

The stem-loop sequence may be processed to the mature sequence of miR-320b. Examples of the mature sequence include those comprising a nucleic acid sequence of miRBase Accession No. MIMAT0005792. For example, the nucleic acid sequence of MIMAT0005792 is as follows: AAAAGCUGGGUUGAGAGGGCAA.

Detection of miR-320b encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-320b can involve detection using one or more probes and/or primers.

miR-320c

The DR diagnostic miRNA miR-320c, which may also be referred to as “hsa-mir-320c” is a member of the miR-320 gene family. Examples of the stem-loop sequence for hsa-mir-320c include those comprising a nucleic acid sequence of miRBase Accession No. MI0003778, MI0008191, or naturally occurring variants thereof. For example, the nucleic acid stem-loop sequence of MI0003778 is as follows: CUUCUCUUUCCAGUUCUUCCCAGAAUUGGGAAAAGCUGGGUUGAGAGG GU; and the nucleic acid stem-loop sequence of MI0008191 is as follows:

CUUCUCUUUCCAGUUCUUCCCAGAAUUGGGAAAAGCUGGGUUGAGAGG
GU.

The stem-loop sequence may be processed to the mature sequence of miR-320c. Examples of the mature sequence include those comprising a nucleic acid sequence of miRBase Accession No. MIMAT0005793. For example, the nucleic acid sequence of MIMAT0005793 is as follows: AAAAGCUGGGUUGAGAGGGU.

Detection of miR-320c encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-320c can involve detection using one or more probes and/or primers.

miR-320e

The DR diagnostic miRNA miR-320e, which may also be referred to as “hsa-mir-320e” is a member of the miR-320 gene family. Examples of the stem-loop sequence for hsa-mir-320e include those comprising a nucleic acid sequence of miRBase Accession No. MI0014234 or naturally occurring variants thereof. For example, the nucleic acid stem-loop sequence of MI0014234 is as follows:

GCCUUCUCUUCCCAGUUCUUCCUGGAGUCGGGGAAAAGCUGGGUUGAG
AAGGU.

The stem-loop sequence may be processed to the mature sequence of miR-320e. Examples of the mature sequence include those comprising a nucleic acid sequence of miRBase Accession No. MIMAT0015072. For example, the nucleic acid sequence of MIMAT0015072 is as follows: AAAGCUGGGUUGAGAAGG.

Detection of miR-320e encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-320e can involve detection using one or more probes and/or primers.

miR-26a

The DR diagnostic miRNA miR-26a, which may also be referred to as “hsa-mir-26a” is a member of the miR-26 gene family. Examples of the stem-loop sequence for hsa-mir-26a include those comprising a nucleic acid sequence of miRBase Accession No. MI0000083, MI0000750, or naturally occurring variants thereof. For example, the nucleic acid stem-loop sequence of MI0000083 is as follows: GUGGCCUCGUUCAAGUAAUCCAGGAUAGGCUGUGCAGGUCCCAAUGGG CCUAUUCUUGGUUACUUGCACGGGGACGC; and the nucleic acid stem-loop sequence of MI0000750 is as follows:

GGCUGUGGCUGGAUUCAAGUAAUCCAGGAUAGGCUGUUUCCAUCUGUG
AGGCCUAUUCUUGAUUACUUGUUUCUGGAGGCAGCU.

The stem-loop sequence may be processed to the mature sequence of miR-26a. Examples of the mature sequence include those comprising a nucleic acid sequence of miRBase Accession No. MIMAT0000082, MIMAT0004499 or MIMAT0004681. For example, the nucleic acid sequence of MIMAT0000082 is as follows: UUCAAGUAAUCCAGGAUAGGCU; the nucleic acid sequence of MIMAT0004499 is as follows: CCUAUUCUUGGUUACUUGCACG; and the nucleic acid sequence of MIMAT0004681 is as follows:

CCUAUUCUUGAUUACUUGUUUC.

Detection of miR-26a encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-26a can involve detection using one or more probes and/or primers.

miR-4488

The DR diagnostic miRNA miR-4488, which may also be referred to as “hsa-mir-4488” is a member of the miR-4488 gene family. Examples of the stem-loop sequence for hsa-mir-4488 include those comprising a nucleic acid sequence of miRBase Accession No. MI0016849 or naturally occurring variants thereof. For example, the nucleic acid stem-loop sequence of MI0016849 is as follows:

GGUAGGGGGCGGGCUCCGGCGCUGGGACCCCACUAGGGUGGCGCCUUG
GCCCCGCCCCGCCC.

The stem-loop sequence may be processed to the mature sequence of miR-4488. Examples of the mature sequence include those comprising a nucleic acid sequence of miRBase Accession No. MIMAT0019022. For example, the nucleic acid sequence of MIMAT0019022 is as follows: AGGGGGCGGGCUCCGGCG.

Detection of miR-4488 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-4488 can involve detection using one or more probes and/or primers.

miR-638

The DR diagnostic miRNA miR-638, which may also be referred to as “hsa-mir-638” is a member of the miR-638 gene family. Examples of the stem-loop sequence for hsa-mir-638 include those comprising a nucleic acid sequence of miRBase Accession No. MI0003653 or naturally occurring variants thereof. For example, the nucleic acid stem-loop sequence of MI0003653 is as follows:

GUGAGCGGGCGCGGCAGGGAUCGCGGGCGGGUGGCGGCCUAGGGCGCG
GAGGGCGGACCGGGAAUGGCGCGCCGUGCGCCGCCGGCGUAACUGCGGC
GCU.

The stem-loop sequence may be processed to the mature sequence of miR-638. Examples of the mature sequence include those comprising a nucleic acid sequence of miRBase Accession No. MIMAT0003308. For example, the nucleic acid sequence of MIMAT0003308 is as follows:

AGGGAUCGCGGGCGGGUGGCGGCCU.

Detection of miR-638 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-638 can involve detection using one or more probes and/or primers.

miR-29a

The DR diagnostic miRNA miR-29a, which may also be referred to as “hsa-mir-29a” is a member of the miR-29 gene family. Examples of the stem-loop sequence for hsa-mir-29a include those comprising a nucleic acid sequence of miRBase Accession No. MI0000087 or naturally occurring variants thereof. For example, the nucleic acid stem-loop sequence of MI0000087 is as follows:

AUGACUGAUUUCUUUUGGUGUUCAGAGUCAAUAUAAUUUUCUAGCACC
AUCUGAAAUCGGUUAU.

The stem-loop sequence may be processed to the mature sequence of miR-29a. Examples of the mature sequence include those comprising a nucleic acid sequence of miRBase Accession No. MIMAT0000086. For example, the nucleic acid sequence of MIMAT0000086 is as follows: UAGCACCAUCUGAAAUCGGUUA.

Detection of miR-29a encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-29a can involve detection using one or more probes and/or primers.

miR-4662a

The DR diagnostic miRNA miR-4662a, which may also be referred to as “hsa-mir-4662a” is a member of the miR-4662 gene family. Examples of the stem-loop sequence for hsa-mir-4662a include those comprising a nucleic acid sequence of miRBase Accession No. MI0017290 or naturally occurring variants thereof. For example, the nucleic acid stem-loop sequence of MI0017290 is as follows:

UCUAUUUAGCCAAUUGUCCAUCUUUAGCUAUUCUGAAUGCCUAAAGAU
AGACAAUUGGCUAAAUAGA.

The stem-loop sequence may be processed to the mature sequence of miR-4662a. Examples of the mature sequence include those comprising a nucleic acid sequence of miRBase Accession No. MIMAT0019731 or MIMAT0019732. For example, the nucleic acid sequence of MIMAT0019731 is as follows: UUAGCCAAUUGUCCAUCUUUAG; and the nucleic acid sequence of MIMAT0019732 is as follows: AAAGAUAGACAAUUGGCUAAAU.

Detection of miR-4662a encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-4662a can involve detection using one or more probes and/or primers.

miR-548

The DR diagnostic miRNA miR-548, which may also be referred to as “hsa-mir-548” is a member of the miR-548 gene family. Examples of the stem-loop sequence for hsa-mir-548 include those comprising a nucleic acid sequence of miRBase Accession No. MI0003630, MI0016813, MI0006374, MI0003596, MI0006395, MI0016851 or naturally occurring variants thereof. For example, the nucleic acid stem-loop sequence of MI0003630 is as follows: CAUUGGCAUCUAUUAGGUUGGUGCAAAAGUAAUUGCGGUUUUUGCCAU UACUUUCAGUAGCAAAAAUCUCAAUUACUUUUGCACCAACUUAAUACU U; the nucleic acid stem-loop sequence of MI0016813 is as follows: GUAUUAGGUUGGUGCAAAGGUAAUUGCAGUUUUUCCCAUUUAAAAUAU GGAAAAAAAAAUCACAAUUACUUUUGCAUCAACCUAAUAA; the nucleic acid stem-loop sequence of MI0006374 is as follows: AUUAGGUUGGUGCAAAAGUAAUCACAGUUUUUGACAUUACUUUCAAAG ACAAAAACUGUAAUUACUUUUGGACCAACCUAAUAG; the nucleic acid stem-loop sequence of MI0003596 is as follows: CAGACUAUAUAUUUAGGUUGGCGCAAAAGUAAUUGUGGUUUUGGCCUU UAUUUUCAAUGGCAAGAACCUCAGUUGCUUUUGUGCCAACCUAAUACU U; the nucleic acid stem-loop sequence of MI0006395 is as follows: AGUUAUUAGAUUAGUGCAAAAGUAAUUGCAGUUUUUGCAUUACGUUCU AUGGCAAAACUGUAAUUACUUUUGUACCAACAUAAUACUUC; and the nucleic acid stem-loop sequence of MI0016851 is as follows:

GGUCGGUGCAAAAGUAAUUGCUGUUUUUGCCAUUAAAAAUAAUGGCAU
UAAAAGUAAUGGCAAAAACGGCAAUGACUUUUGUACCAAUCUAAUAUC
U.

The stem-loop sequence may be processed to the mature sequence of miR-548. Examples of the mature sequence include those comprising a nucleic acid sequence of miRBase Accession No. MIMAT0003285, MIMAT0018989, MIMAT0026739, MIMAT0005895, MIMAT0003254, MIMAT0022722, MIMAT0005912, or MIMAT0019024. For example, the nucleic acid sequence of MIMAT0003285 is as follows: CAAAAAUCUCAAUUACUUUUGC; the nucleic acid sequence of MIMAT0018989 is as follows: AAAGGUAAUUGCAGUUUUUCCC; the nucleic acid sequence of MIMAT0026739 is as follows: UGCAAAAGUAAUCACAGUUUUU; the nucleic acid sequence of MIMAT0005895 is as follows: AAAAACUGUAAUUACUUUU; the nucleic acid sequence of MIMAT0003254 is as follows: CAAGAACCUCAGUUGCUUUUGU; the nucleic acid sequence of MIMAT0022722 is as follows: UGCAAAAGUAAUUGCAGUUUUUG; the nucleic acid sequence of MIMAT0005912 is as follows: AAAACUGUAAUUACUUUUGUAC; and the nucleic acid sequence of MIMAT0019024 is as follows:

AACGGCAAUGACUUUUGUACCA.

Detection of miR-548 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-548 can involve detection using one or more probes and/or primers.

### Methods of Determining a Likelihood of Having or Developing Dry or Wet AMD

As summarized above, aspects of the present disclosure include methods for determining a likelihood of having or developing dry or wet AMD in a subject. Such a determination of the likelihood of having or developing dry or wet AMD may include computing a likelihood of having or developing dry or wet AMD in the subject so as to differentiate the subject from a healthy individual. A determination of the likelihood of having or developing dry or wet AMD can involve differentiating the subject from an individual having dry or wet AMD.

In general, the methods involve determining an amount of at least one dry or wet AMD diagnostic miRNA in a biological sample from the subject; comparing the amount of the at least one dry or wet AMD diagnostic miRNA with a control amount of the at least one dry or wet AMD diagnostic miRNA; and generating a report indicating a likelihood of having or developing dry or wet AMD in the subject based on results of said comparing the amount of the at least one dry or wet AMD diagnostic miRNA with the control amount of the at least one dry or wet AMD diagnostic miRNA. The detection of one or more such dry or wet AMD diagnostic miRNAs can be used to determine a likelihood of having or developing dry or wet AMD in a subject. The methods of the present disclosure also find use in facilitating treatment decisions for a subject.

The dry or wet AMD diagnostic miRNAs used in the methods of the present disclosure, as well as the methods of detection and analysis, are described in more detail below.

Wet AMD Diagnostic miRNAs for Detection

In some embodiments, the methods of present disclosure involve detection of a wet AMD diagnostic miRNA in a biological sample of a patient. Specifically, in some embodiments, the present methods involve detection of one or more of the miRNAs identified herein (or a precursor or derivative thereof) as being differentially present in a sample taken from a subject having wet AMD as compared with a subject not having wet AMD. Such miRNAs include, e.g., miR-10b, miR-1306, miR-136, miR-183-star, miR-296-5p, miR-30c, miR-4258, miR-519d, miR-889, and miR-486.

In certain embodiments, the methods involve detection of or determination of an amount of one of miR-10b, miR-1306, miR-136, miR-183-star, miR-296-5p, miR-30c, miR-4258, miR-519d, miR-889, and miR-486. In certain embodiments, the methods involve detection of or determination of an amount of any two of miR-10b, miR-1306, miR-136, miR-183-star, miR-296-5p, miR-30c, miR-4258, miR-519d, miR-889, and miR-486. In certain embodiments, the methods involve detection of or determination of an amount of any three of miR-10b, miR-1306, miR-136, miR-183-star, miR-296-5p, miR-30c, miR-4258, miR-519d, miR-889, and miR-486. In certain embodiments, the methods involve detection of or determination of an amount of any four of miR-10b, miR-1306, miR-136, miR-183-star, miR-296-5p, miR-30c, miR-4258, miR-519d, miR-889, and miR-486. In certain embodiments, the methods involve detection of or determination of an amount of any five of miR-10b, miR-1306, miR-136, miR-183-star, miR-296-5p, miR-30c, miR-4258, miR-519d, miR-889, and miR-486. In certain embodiments, the methods involve detection of or determination of an amount of any six of miR-10b, miR-1306, miR-136, miR-183-star, miR-296-5p, miR-30c, miR-4258, miR-519d, miR-889, and miR-486. In certain embodiments, the methods involve detection of or determination of an amount of any seven of miR-10b, miR-1306, miR-136, miR-183-star, miR-296-5p, miR-30c, miR-4258, miR-519d, miR-889, and miR-486. In certain embodiments, the methods involve detection of or determination of an amount of any eight of miR-10b, miR-1306, miR-136, miR-183-star, miR-296-5p, miR-30c, miR-4258, miR-519d, miR-889, and miR-486. In certain embodiments, the methods involve detection of or determination of an amount of any nine of miR-10b, miR-1306, miR-136, miR-183-star, miR-296-5p, miR-30c, miR-4258, miR-519d, miR-889, and miR-486. In certain embodiments, the methods involve detection of or determination of an amount of all ten of miR-10b, miR-1306, miR-136, miR-183-star, miR-296-5p, miR-30c, miR-4258, miR-519d, miR-889, and miR-486. The methods can further involve detection of other biomarkers.

miR-10b

The wet AMD diagnostic miRNA miR-10b, which may also be referred to as “hsa-mir-10b” is a member of the miR-10 gene family. Examples of the stem-loop sequence for hsa-mir-10b include those comprising a nucleic acid sequence of miRBase Accession No. MI0000267 or naturally occurring variants thereof. For example, the nucleic acid stem-loop sequence of MI0000267 is as follows:

CCAGAGGUUGUAACGUUGUCUAUAUAUACCCUGUAGAACCGAAUUUGU
GUGGUAUCCGUAUAGUCACAGAUUCGAUUCUAGGGGAAUAUAUGGUCG
AUGCAAAAACUUCA.

The stem-loop sequence may be processed to the mature sequence of miR-10b. Examples of the mature sequence include those comprising a nucleic acid sequence of miRBase Accession No. MIMAT0000254 or MIMAT0004556. For example, the nucleic acid sequence of MIMAT0000254 is as follows: UACCCUGUAGAACCGAAUUUGUG; and the nucleic acid sequence of MIMAT0004556 is as follows: ACAGAUUCGAUUCUAGGGGAAU.

Detection of miR-10b encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-10b can involve detection using one or more probes and/or primers.

miR-1306

The wet AMD diagnostic miRNA miR-1306, which may also be referred to as “hsa-mir-1306” is a member of the miR-1306 gene family. Examples of the stem-loop sequence for hsa-mir-1306 include those comprising a nucleic acid sequence of miRBase Accession No. MI0006443 or naturally occurring variants thereof. For example, the nucleic acid stem-loop sequence of MI0006443 is as follows:

GUGAGCAGUCUCCACCACCUCCCCUGCAAACGUCCAGUGGUGCAGAGGU
AAUGGACGUUGGCUCUGGUGGUGAUGGACAGUCCGA.

The stem-loop sequence may be processed to the mature sequence of miR-1306. Examples of the mature sequence include those comprising a nucleic acid sequence of miRBase Accession No. MIMAT0022726 or MIMAT0005950. For example, the nucleic acid sequence of MIMAT0022726 is as follows: CCACCUCCCCUGCAAACGUCCA; and the nucleic acid sequence of MIMAT0005950 is as follows: ACGUUGGCUCUGGUGGUG.

Detection of miR-1306 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-1306 can involve detection using one or more probes and/or primers.

miR-136

The wet AMD diagnostic miRNA miR-136, which may also be referred to as “hsa-mir-136” is a member of the miR-136 gene family. Examples of the stem-loop sequence for hsa-mir-136 include those comprising a nucleic acid sequence of miRBase Accession No. MI0000475 or naturally occurring variants thereof. For example, the nucleic acid stem-loop sequence of MI0000475 is as follows:

UGAGCCCUCGGAGGACUCCAUUUGUUUUGAUGAUGGAUUCUUAUGCUC
CAUCAUCGUCUCAAAUGAGUCUUCAGAGGGUUCU.

The stem-loop sequence may be processed to the mature sequence of miR-136. Examples of the mature sequence include those comprising a nucleic acid sequence of miRBase Accession No. MIMAT0000448 or MIMAT0004606. For example, the nucleic acid sequence of MIMAT0000448 is as follows: ACUCCAUUUGUUUUGAUGAUGGA; and the nucleic acid sequence of MIMAT0004606 is as follows: CAUCAUCGUCUCAAAUGAGUCU.

Detection of miR-136 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-136 can involve detection using one or more probes and/or primers.

miR-183-star

The wet AMD diagnostic miRNA miR-183-star, which may also be referred to as “hsa-mir-183-star” is a member of the miR-183 gene family. Examples of the stem-loop sequence for hsa-mir-183 include those comprising a nucleic acid sequence of miRBase Accession No. MI0000273 or naturally occurring variants thereof. For example, the nucleic acid stem-loop sequence of MI0000273 is as follows:

CCGCAGAGUGUGACUCCUGUUCUGUGUAUGGCACUGGUAGAAUUCACU
GUGAACAGUCUCAGUCAGUGAAUUACCGAAGGGCCAUAAACAGAGCAG
AGACAGAUCCACGA.

The stem-loop sequence may be processed to the mature sequence of miR-183 and miR-183-star. Examples of the mature sequence include those comprising a nucleic acid sequence of miRBase Accession No. MIMAT0000261 or MIMAT0004560. For example, the nucleic acid sequence of MIMAT0000261 is as follows: UAUGGCACUGGUAGAAUUCACU; and the nucleic acid sequence of MIMAT0004560 is as follows: GUGAAUUACCGAAGGGCCAUAA.

Detection of miR-183-star encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-183-star can involve detection using one or more probes and/or primers.

miR-296-5p

The wet AMD diagnostic miRNA miR-296-5p, which may also be referred to as “hsa-mir-296-5p” is a member of the miR-296 gene family. Examples of the stem-loop sequence for hsa-mir-296 include those comprising a nucleic acid sequence of miRBase Accession No. MI0000747 or naturally occurring variants thereof. For example, the nucleic acid stem-loop sequence of MI0000747 is as follows:

AGGACCCUUCCAGAGGGCCCCCCCUCAAUCCUGUUGUGCCUAAUUCAGA
GGGUUGGGUGGAGGCUCUCCUGAAGGGCUCU.

The stem-loop sequence may be processed to the mature sequence of miR-296-5p. Examples of the mature sequence include those comprising a nucleic acid sequence of miRBase Accession No. MIMAT0000690. For example, the nucleic acid sequence of MIMAT0000690 is as follows: AGGGCCCCCCCUCAAUCCUGU.

Detection of miR-296-5p encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-296-5p can involve detection using one or more probes and/or primers.

miR-30c

The wet AMD diagnostic miRNA miR-30c, which may also be referred to as “hsa-mir-30c” is a member of the miR-30 gene family. Examples of the stem-loop sequence for hsa-mir-30c include those comprising a nucleic acid sequence of miRBase Accession No. MI0000736, MI0000254, or naturally occurring variants thereof. For example, the nucleic acid stem-loop sequence of MI0000736 is as follows: ACCAUGCUGUAGUGUGUGUAAACAUCCUACACUCUCAGCUGUGAGCUC AAGGUGGCUGGGAGAGGGUUGUUUACUCCUUCUGCCAUGGA; and the nucleic acid stem-loop sequence of MI0000254 is as follows:

AGAUACUGUAAACAUCCUACACUCUCAGCUGUGGAAAGUAAGAAAGCU
GGGAGAAGGCUGUUUACUCUUUCU.

The stem-loop sequence may be processed to the mature sequence of miR-30c. Examples of the mature sequence include those comprising a nucleic acid sequence of miRBase Accession No. MIMAT0000244, MIMAT0004674, or MIMAT0004550. For example, the nucleic acid sequence of MIMAT0000244 is as follows: UGUAAACAUCCUACACUCUCAGC; the nucleic acid sequence of MIMAT0004674 is as follows: CUGGGAGAGGGUUGUUUACUCC; and the nucleic acid sequence of MIMAT0004550 is as follows:

CUGGGAGAAGGCUGUUUACUCU.

Detection of miR-30c encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-30c can involve detection using one or more probes and/or primers.

miR-4258

The wet AMD diagnostic miRNA miR-4258 may also be referred to as “hsa-mir-4258.” Examples of the stem-loop sequence for miR-4258 include those comprising a nucleic acid sequence of miRBase Accession No. MI0015857 or naturally occurring variants thereof. For example, the nucleic acid stem-loop sequence of MI0015857 is as follows:

ACGCCCCCCGCCCCGCCACCGCCUUGGAGGCUGACCUCUUACUUUCGGU
CGGUCUUCUUCCCUGGGCUUGGUUUGGGGGCGGGGGAGUGUC.

The stem-loop sequence may be processed to the mature sequence of miR-4258. Examples of the mature sequence include those comprising a nucleic acid sequence of miRBase Accession No. MIMAT0016879. For example, the nucleic acid sequence of MIMAT0016879 is as follows: CCCCGCCACCGCCUUGG.

Detection of miR-4258 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-4258 can involve detection using one or more probes and/or primers.

miR-519d

The wet AMD diagnostic miRNA miR-519d may also be referred to as “hsa-mir-519d.” Examples of the stem-loop sequence for miR-519d include those comprising a nucleic acid sequence of miRBase Accession No. MI0003162 or naturally occurring variants thereof. For example, the nucleic acid stem-loop sequence of MI0003162 is as follows:

UCCCAUGCUGUGACCCUCCAAAGGGAAGCGCUUUCUGUUUGUUUUCUC
UUAAACAAAGUGCCUCCCUUUAGAGUGUUACCGUUUGGGA.

The stem-loop sequence may be processed to the mature sequence of miR-519d. Examples of the mature sequence include those comprising a nucleic acid sequence of miRBase Accession No. MIMAT0026610 or MIMAT0002853. For example, the nucleic acid sequence of MIMAT0026610 is as follows: CCUCCAAAGGGAAGCGCUUUCUGUU; and the nucleic acid sequence of MIMAT0002853 is as follows CAAAGUGCCUCCCUUUAGAGUG.

Detection of miR-519d encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-519d can involve detection using one or more probes and/or primers.

miR-889

The wet AMD diagnostic miRNA miR-889 may also be referred to as “hsa-mir-889.” Examples of the stem-loop sequence for miR-889 include those comprising a nucleic acid sequence of miRBase Accession No. MI0005540 or naturally occurring variants thereof. For example, the nucleic acid stem-loop sequence of MI0005540 is as follows:

GUGCUUAAAGAAUGGCUGUCCGUAGUAUGGUCUCUAUAUUUAUGAUGA
UUAAUAUCGGACAACCAUUGUUUUAGUAUCC.

The stem-loop sequence may be processed to the mature sequence of miR-889. Examples of the mature sequence include those comprising a nucleic acid sequence of miRBase Accession No. MIMAT0026719 or MIMAT0004921. For example, the nucleic acid sequence of MIMAT0026719 is as follows: AAUGGCUGUCCGUAGUAUGGUC; and the nucleic acid sequence of MIMAT0004921 is as follows: UUAAUAUCGGACAACCAUUGU.

Detection of miR-889 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-889 can involve detection using one or more probes and/or primers.

miR-486

The wet AMD diagnostic miRNA miR-486, which may also be referred to as “hsa-miR-486” is a member of the miR-486 gene family. Examples of the stem-loop sequence for hsa-mir-486 include those comprising a nucleic acid sequence of miRBase Accession No. MI0002470 or naturally occurring variants thereof. For example, the nucleic acid stem-loop sequence of MI0002470 is as follows:

GCAUCCUGUACUGAGCUGCCCCGAGGCCCUUCAUGCUGCCCAGCUCGGG
GCAGCUCAGUACAGGAUAC.

The stem-loop sequence may be processed to the mature sequence of miR-486. Examples of the mature sequence include those comprising a nucleic acid sequence of miRBase Accession No. MIMAT0002177 or MIMAT0004762. For example, the nucleic acid sequence of MIMAT0002177 is as follows: UCCUGUACUGAGCUGCCCCGAG; and the nucleic acid sequence of MIMAT0004762 is as follows: CGGGGCAGCUCAGUACAGGAU.

Detection of miR-486 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-486 can involve detection using one or more probes and/or primers.

Dry AMD Diagnostic miRNAs for Detection

In some embodiments, the methods of present disclosure involve detection of a dry AMD diagnostic miRNA in a biological sample of a patient. Specifically, in some embodiments, the present methods involve detection of one or more of the miRNAs identified herein (or a precursor or derivative thereof) as being differentially present in a sample taken from a subject having dry AMD as compared with a subject not having dry AMD. Such miRNAs include, e.g., miR-1224-3p, miR-1226, miR-29c, miR-3121, miR-3197, miR-32, miR-363-star, miR-639, miR-661, miR-708-star, and miR-486.

In certain embodiments, the methods involve detection of or determination of an amount of one of miR-1224-3p, miR-1226, miR-29c, miR-3121, miR-3197, miR-32, miR-363-star, miR-639, miR-661, miR-708-star, and miR-486. In certain embodiments, the methods involve detection of or determination of an amount of any two of miR-1224-3p, miR-1226, miR-29c, miR-3121, miR-3197, miR-32, miR-363-star, miR-639, miR-661, miR-708-star, and miR-486. In certain embodiments, the methods involve detection of or determination of an amount of any three of miR-1224-3p, miR-1226, miR-29c, miR-3121, miR-3197, miR-32, miR-363-star, miR-639, miR-661, miR-708-star, and miR-486. In certain embodiments, the methods involve detection of or determination of an amount of any four of miR-1224-3p, miR-1226, miR-29c, miR-3121, miR-3197, miR-32, miR-363-star, miR-639, miR-661, miR-708-star, and miR-486. In certain embodiments, the methods involve detection of or determination of an amount of any five of miR-1224-3p, miR-1226, miR-29c, miR-3121, miR-3197, miR-32, miR-363-star, miR-639, miR-661, miR-708-star, and miR-486. In certain embodiments, the methods involve detection of or determination of an amount of any six of miR-1224-3p, miR-1226, miR-29c, miR-3121, miR-3197, miR-32, miR-363-star, miR-639, miR-661, miR-708-star, and miR-486. In certain embodiments, the methods involve detection of or determination of an amount of any seven of miR-1224-3p, miR-1226, miR-29c, miR-3121, miR-3197, miR-32, miR-363-star, miR-639, miR-661, miR-708-star, and miR-486. In certain embodiments, the methods involve detection of or determination of an amount of any eight of miR-1224-3p, miR-1226, miR-29c, miR-3121, miR-3197, miR-32, miR-363-star, miR-639, miR-661, miR-708-star, and miR-486. In certain embodiments, the methods involve detection of or determination of an amount of any nine of miR-1224-3p, miR-1226, miR-29c, miR-3121, miR-3197, miR-32, miR-363-star, miR-639, miR-661, miR-708-star, and miR-486. In certain embodiments, the methods involve detection of or determination of an amount of any ten of miR-1224-3p, miR-1226, miR-29c, miR-3121, miR-3197, miR-32, miR-363-star, miR-639, miR-661, miR-708-star, and miR-486. In certain embodiments, the methods involve detection of or determination of an amount of all eleven of miR-1224-3p, miR-1226, miR-29c, miR-3121, miR-3197, miR-32, miR-363-star, miR-639, miR-661, miR-708-star, and miR-486. The methods can further involve detection of other biomarkers.

miR-1224-3p

The dry AMD diagnostic miRNA miR-1224-3p is also referred to as “hsa-mir-1224-3p”. Examples of the stem-loop sequence for hsa-mir-1224-3p include those comprising a nucleic acid sequence of miRBase Accession No. MI0003764 or naturally occurring variants thereof. For example, the nucleic acid stem-loop sequence of MI0003764 is as follows:

GUGAGGACUCGGGAGGUGGAGGGUGGUGCCGCCGGGGCCGGGCGCUGU
UUCAGCUCGCUUCUCCCCCCACCUCCUCUCUCCUCAG.

The stem-loop sequence may be processed to the mature sequence of miR-1224-3p. Examples of the mature sequence include those comprising a nucleic acid sequence of miRBase Accession No. MIMAT0005459. For example, the nucleic acid sequence of MIMAT0005459 is as follows: CCCCACCUCCUCUCUCCUCAG.

Detection of miR-1224-3p encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-1224-3p can involve detection using one or more probes and/or primers.

miR-1226

The dry AMD diagnostic miRNA miR-1226 is also referred to as “hsa-mir-1226”. Examples of the stem-loop sequence for hsa-mir-1226 include those comprising a nucleic acid sequence of miRBase Accession No. MI0006313 or naturally occurring variants thereof. For example, the nucleic acid stem-loop sequence of MI0006313 is as follows:

GUGAGGGCAUGCAGGCCUGGAUGGGGCAGCUGGGAUGGUCCAAAAGGG
UGGCCUCACCAGCCCUGUGUUCCCUAG.

The stem-loop sequence may be processed to the mature sequence of miR-1226. Examples of the mature sequence include those comprising a nucleic acid sequence of miRBase Accession No. MIMAT0005576 or MIMAT0005577. For example, the nucleic acid sequence of MIMAT0005576 is as follows: GUGAGGGCAUGCAGGCCUGGAUGGGG; and the nucleic acid sequence of MIMAT0005577 is as follows: UCACCAGCCCUGUGUUCCCUAG.

Detection of miR-1226 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-1226 can involve detection using one or more probes and/or primers.

miR-29c

The dry AMD diagnostic miRNA miR-29c is also referred to as “hsa-mir-29c”. Examples of the stem-loop sequence for hsa-mir-29c include those comprising a nucleic acid sequence of miRBase Accession No. MI0000735 or naturally occurring variants thereof. For example, the nucleic acid stem-loop sequence of MI0000735 is as follows:

AUCUCUUACACAGGCUGACCGAUUUCUCCUGGUGUUCAGAGUCUGUUU
UUGUCUAGCACCAUUUGAAAUCGGUUAUGAUGUAGGGGGA.

The stem-loop sequence may be processed to the mature sequence of miR-29c. Examples of the mature sequence include those comprising a nucleic acid sequence of miRBase Accession No. MIMAT0004673 or MIMAT0000681. For example, the nucleic acid sequence of MIMAT0004673 is as follows: UGACCGAUUUCUCCUGGUGUUC; and the nucleic acid sequence of MIMAT0000681 is as follows: UAGCACCAUUUGAAAUCGGUUA.

Detection of miR-29c encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-29c can involve detection using one or more probes and/or primers.

miR-3121

The dry AMD diagnostic miRNA miR-3121 is also referred to as “hsa-mir-3121”. Examples of the stem-loop sequence for hsa-mir-3121 include those comprising a nucleic acid sequence of miRBase Accession No. MI0014137 or naturally occurring variants thereof. For example, the nucleic acid stem-loop sequence of MI0014137 is as follows:

AAAUGGUUAUGUCCUUUGCCUAUUCUAUUUAAGACACCCUGUACCUUA
AAUAGAGUAGGCAAAGGACAGAAACAUUU.

The stem-loop sequence may be processed to the mature sequence of miR-3121. Examples of the mature sequence include those comprising a nucleic acid sequence of miRBase Accession No. MIMAT0019199 or MIMAT0014983. For example, the nucleic acid sequence of MIMAT0019199 is as follows: UCCUUUGCCUAUUCUAUUUAAG; and the nucleic acid sequence of MIMAT0014983 is as follows: UAAAUAGAGUAGGCAAAGGACA.

Detection of miR-3121 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-3121 can involve detection using one or more probes and/or primers.

miR-3197

The dry AMD diagnostic miRNA miR-3197 is also referred to as “hsa-mir-3197”. Examples of the stem-loop sequence for hsa-mir-3197 include those comprising a nucleic acid sequence of miRBase Accession No. MI0014245 or naturally occurring variants thereof. For example, the nucleic acid stem-loop sequence of MI0014245 is as follows:

GGCGAGGGGAGGCGCAGGCUCGGAAAGGCGCGCGAGGCUCCAGGCUCC
UUCCCGAUCCACCGCUCUCCUCGCU.

The stem-loop sequence may be processed to the mature sequence of miR-3197. Examples of the mature sequence include those comprising a nucleic acid sequence of miRBase Accession No. MIMAT0015082. For example, the nucleic acid sequence of MIMAT0015082 is as follows: GGAGGCGCAGGCUCGGAAAGGCG.

Detection of miR-3197 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-3197 can involve detection using one or more probes and/or primers.

miR-32

The dry AMD diagnostic miRNA miR-32 is also referred to as “hsa-mir-32”. Examples of the stem-loop sequence for hsa-mir-32 include those comprising a nucleic acid sequence of miRBase Accession No. MI0000090 or naturally occurring variants thereof. For example, the nucleic acid stem-loop sequence of MI0000090 is as follows:

GGAGAUAUUGCACAUUACUAAGUUGCAUGUUGUCACGGCCUCAAUGCA
AUUUAGUGUGUGUGAUAUUUUC.

The stem-loop sequence may be processed to the mature sequence of miR-32. Examples of the mature sequence include those comprising a nucleic acid sequence of miRBase Accession No. MIMAT0000090 or MIMAT0004505. For example, the nucleic acid sequence of MIMAT0000090 is as follows: UAUUGCACAUUACUAAGUUGCA; and the nucleic acid sequence of MIMAT0004505 is as follows: CAAUUUAGUGUGUGUGAUAUUU.

Detection of miR-32 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-32 can involve detection using one or more probes and/or primers.

miR-363-star

The dry AMD diagnostic miRNA miR-363-star is also referred to as “hsa-mir-363”. Examples of the stem-loop sequence for hsa-mir-363 include those comprising a nucleic acid sequence of miRBase Accession No. MI0000764 or naturally occurring variants thereof. For example, the nucleic acid stem-loop sequence of MI0000764 is as follows:

UGUUGUCGGGUGGAUCACGAUGCAAUUUUGAUGAGUAUCAUAGGAGAA
AAAUUGCACGGUAUCCAUCUGUAAACC.

The stem-loop sequence may be processed to the mature sequence of miR-363 or miR-363-star. Examples of the mature sequence include those comprising a nucleic acid sequence of miRBase Accession No. MIMAT0003385 or MIMAT0000707. For example, the nucleic acid sequence of MIMAT0003385 is as follows: CGGGUGGAUCACGAUGCAAUUU; and the nucleic acid sequence of MIMAT0000707 is as follows: AAUUGCACGGUAUCCAUCUGUA.

Detection of miR-363-star encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-363-star can involve detection using one or more probes and/or primers.

miR-639

The dry AMD diagnostic miRNA miR-639 is also referred to as “hsa-mir-639”. Examples of the stem-loop sequence for hsa-mir-639 include those comprising a nucleic acid sequence of miRBase Accession No. MI0003654 or naturally occurring variants thereof. For example, the nucleic acid stem-loop sequence of MI0003654 is as follows:

UGGCCGACGGGGCGCGCGCGGCCUGGAGGGGCGGGGCGGACGCAGAGCC
GCGUUUAGUCUAUCGCUGCGGUUGCGAGCGCUGUAGGGAGCCUGUGCU
G.

The stem-loop sequence may be processed to the mature sequence of miR-639. Examples of the mature sequence include those comprising a nucleic acid sequence of miRBase Accession No. MIMAT0003309. For example, the nucleic acid sequence of MIMAT0003309 is as follows: AUCGCUGCGGUUGCGAGCGCUGU.

Detection of miR-639 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-639 can involve detection using one or more probes and/or primers.

miR-661

The dry AMD diagnostic miRNA miR-661 is also referred to as “hsa-mir-661”. Examples of the stem-loop sequence for hsa-mir-661 include those comprising a nucleic acid sequence of miRBase Accession No. MI0003669 or naturally occurring variants thereof. For example, the nucleic acid stem-loop sequence of MI0003669 is as follows:

GGAGAGGCUGUGCUGUGGGGCAGGCGCAGGCCUGAGCCCUGGUUUCGG
GCUGCCUGGGUCUCUGGCCUGCGCGUGACUUUGGGGUGGCU.

The stem-loop sequence may be processed to the mature sequence of miR-661. Examples of the mature sequence include those comprising a nucleic acid sequence of miRBase Accession No. MIMAT0003324. For example, the nucleic acid sequence of MIMAT0003324 is as follows:

UGCCUGGGUCUCUGGCCUGCGCGU.

Detection of miR-661 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-661 can involve detection using one or more probes and/or primers.

miR-708-star

The dry AMD diagnostic miRNA miR-708-star is also referred to as “hsa-mir-708”. Examples of the stem-loop sequence for hsa-mir-708 include those comprising a nucleic acid sequence of miRBase Accession No. MI0005543 or naturally occurring variants thereof. For example, the nucleic acid stem-loop sequence of MI0005543 is as follows:

AACUGCCCUCAAGGAGCUUACAAUCUAGCUGGGGGUAAAUGACUUGCA
CAUGAACACAACUAGACUGUGAGCUUCUAGAGGGCAGGGA.

The stem-loop sequence may be processed to the mature sequence of miR-708 or miR-708-star. Examples of the mature sequence include those comprising a nucleic acid sequence of miRBase Accession No. MIMAT0004926 or MIMAT0004927. For example, the nucleic acid sequence of MIMAT0004926 is as follows: AAGGAGCUUACAAUCUAGCUGGG; and the nucleic acid sequence of MIMAT0004927 is as follows: CAACUAGACUGUGAGCUUCUAG.

Detection of miR-708-star encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-708-star can involve detection using one or more probes and/or primers.

miR-486

The dry AMD diagnostic miRNA miR-486, which may also be referred to as “hsa-miR-486” is a member of the miR-486 gene family. Examples of the stem-loop sequence for hsa-mir-486 include those comprising a nucleic acid sequence of miRBase Accession No. MI0002470 or naturally occurring variants thereof. For example, the nucleic acid stem-loop sequence of MI0002470 is as follows:

GCAUCCUGUACUGAGCUGCCCCGAGGCCCUUCAUGCUGCCCAGCUCGGGG
CAGCUCAGUACAGGAUAC.

The stem-loop sequence may be processed to the mature sequence of miR-486. Examples of the mature sequence include those comprising a nucleic acid sequence of miRBase Accession No. MIMAT0002177 or MIMAT0004762. For example, the nucleic acid sequence of MIMAT0002177 is as follows: UCCUGUACUGAGCUGCCCCGAG; and the nucleic acid sequence of MIMAT0004762 is as follows: CGGGGCAGCUCAGUACAGGAU.

Detection of miR-486 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-486 can involve detection using one or more probes and/or primers.

### Methods of Determining a Likelihood of Having or Developing Resistance to Anti-VEGF Therapy

As summarized above, aspects of the present disclosure include methods for determining a likelihood of having or developing a resistance to anti-VEGF therapy in a subject suffering from age related macular degeneration (AMD). Such a determination of the likelihood of having or developing a resistance to anti-VEGF therapy may include computing a likelihood of having or developing a resistance to anti-VEGF therapy in the subject so as to differentiate the subject from a healthy individual or an individual suffering from AMD who does not have a resistance to anti-VEGF therapy. A determination of the likelihood of having or developing a resistance to anti-VEGF therapy in a subject can involve differentiating the subject from an individual having a resistance to anti-VEGF therapy.

In general, the methods involve determining an amount of at least one anti-VEGF therapy resistance diagnostic miRNA in a biological sample from the subject; comparing the amount of the at least one anti-VEGF therapy resistance diagnostic miRNA with a control amount of the at least one anti-VEGF therapy resistance diagnostic miRNA; and generating a report indicating a likelihood of having or developing anti-VEGF therapy resistance in the subject based on results of said comparing the amount of the at least one anti-VEGF therapy resistance diagnostic miRNA with the control amount of the at least one anti-VEGF therapy resistance diagnostic miRNA. The detection of one or more such anti-VEGF therapy resistance diagnostic miRNAs can be used to determine a likelihood of having or developing a resistance to anti-VEGF treatment in a subject. The methods of the present disclosure also find use in facilitating treatment decisions for a subject.

The anti-VEGF therapy resistance diagnostic miRNAs used in the methods of the present disclosure, as well as the methods of detection and analysis, are described in more detail below.

Anti-VEGF Therapy Resistance Diagnostic miRNAs for Detection

In some embodiments, the methods of present disclosure involve detection of an anti-VEGF therapy resistance diagnostic miRNA in a biological sample of a patient. Specifically, in some embodiments, the present methods involve detection of one or more of the miRNAs identified herein (or a precursor or derivative thereof) as being differentially present in a sample taken from a subject resistant to anti-VEGF therapy as compared with a subject not resistant to anti-VEGF therapy. Such miRNAs include, e.g., miR-1273, miR-302f, miR-30c, miR-3161, miR-3197, miR-483-3p, miR-550-star, miR-573, miR-577, miR-let-7b and miR-486.

In certain embodiments, the methods involve detection of or determination of an amount of one of miR-1273, miR-302f, miR-30c, miR-3161, miR-3197, miR-483-3p, miR-550-star, miR-573, miR-577, miR-let-7b and miR-486. In certain embodiments, the methods involve detection of or determination of an amount of any two of miR-1273, miR-302f, miR-30c, miR-3161, miR-3197, miR-483-3p, miR-550-star, miR-573, miR-577, miR-let-7b and miR-486. In certain embodiments, the methods involve detection of or determination of an amount of any three of miR-1273, miR-302f, miR-30c, miR-3161, miR-3197, miR-483-3p, miR-550-star, miR-573, miR-577, miR-let-7b and miR-486. In certain embodiments, the methods involve detection of or determination of an amount of any four of miR-1273, miR-302f, miR-30c, miR-3161, miR-3197, miR-483-3p, miR-550-star, miR-573, miR-577, miR-let-7b and miR-486. In certain embodiments, the methods involve detection of or determination of an amount of any five of miR-1273, miR-302f, miR-30c, miR-3161, miR-3197, miR-483-3p, miR-550-star, miR-573, miR-577, miR-let-7b and miR-486. In certain embodiments, the methods involve detection of or determination of an amount of any six of miR-1273, miR-302f, miR-30c, miR-3161, miR-3197, miR-483-3p, miR-550-star, miR-573, miR-577, miR-let-7b and miR-486. In certain embodiments, the methods involve detection of or determination of an amount of any seven of miR-1273, miR-302f, miR-30c, miR-3161, miR-3197, miR-483-3p, miR-550-star, miR-573, miR-577, miR-let-7b and miR-486. In certain embodiments, the methods involve detection of or determination of an amount of any eight of miR-1273, miR-302f, miR-30c, miR-3161, miR-3197, miR-483-3p, miR-550-star, miR-573, miR-577, miR-let-7b and miR-486. In certain embodiments, the methods involve detection of or determination of an amount of any nine of miR-1273, miR-302f, miR-30c, miR-3161, miR-3197, miR-483-3p, miR-550-star, miR-573, miR-577, miR-let-7b and miR-486. In certain embodiments, the methods involve detection of or determination of an amount of any ten of miR-1273, miR-302f, miR-30c, miR-3161, miR-3197, miR-483-3p, miR-550-star, miR-573, miR-577, miR-let-7b and miR-486. In certain embodiments, the methods involve detection of or determination of an amount of all eleven of miR-1273, miR-302f, miR-30c, miR-3161, miR-3197, miR-483-3p, miR-550-star, miR-573, miR-577, miR-let-7b and miR-486. The methods can further involve detection of other biomarkers.

miR-1273

The anti-VEGF therapy resistance diagnostic miRNA miR-1273 is also referred to as “hsa-mir-1273”. Examples of the stem-loop sequence for hsa-mir-1273 include those comprising a nucleic acid sequence of miRBase Accession No. MI0006409 or naturally occurring variants thereof. For example, the nucleic acid stem-loop sequence of MI0006409 is as follows:

UGAGGCAGGAGAAUUGCUUGAACCCGGGUGGUGGAGGUUGCAGUGAGCCA
AGAUUGCGCCACUGCACUCCAGCCUGGGCGACAAAGCAAGACUCUUUCUU
GGA.

The stem-loop sequence may be processed to the mature sequence of miR-1273. Examples of the mature sequence include those comprising a nucleic acid sequence of miRBase Accession No. MIMAT0005926. For example, the nucleic acid sequence of MIMAT0005926 is as follows:

GGGCGACAAAGCAAGACUCUUUCUU.

Detection of miR-1273 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-1273 can involve detection using one or more probes and/or primers.

miR-302f

The anti-VEGF therapy resistance diagnostic miRNA miR-302f is also referred to as “hsa-mir-302f”. Examples of the stem-loop sequence for hsa-mir-302f include those comprising a nucleic acid sequence of miRBase Accession No. MI0006418 or naturally occurring variants thereof. For example, the nucleic acid stem-loop sequence of MI0006418 is as follows:

UCUGUGUAAACCUGGCAAUUUUCACUUAAUUGCUUCCAUGUUUAUAAAAG
A.

The stem-loop sequence may be processed to the mature sequence of miR-302f Examples of the mature sequence include those comprising a nucleic acid sequence of miRBase Accession No. MIMAT0005932. For example, the nucleic acid sequence of MIMAT0005932 is as follows: UAAUUGCUUCCAUGUUU.

Detection of miR-302f encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-302f can involve detection using one or more probes and/or primers.

miR-30c

The anti-VEGF therapy resistance diagnostic miRNA miR-30c is also referred to as “hsa-mir-30c”. Examples of the stem-loop sequence for hsa-mir-30c include those comprising a nucleic acid sequence of miRBase Accession No. MI0000254 or naturally occurring variants thereof. For example, the nucleic acid stem-loop sequence of MI0000254 is as follows:

AGAUACUGUAAACAUCCUACACUCUCAGCUGUGGAAAGUAAGAAAGCUGG
GAGAAGGCUGUUUACUCUUUCU.

The stem-loop sequence may be processed to the mature sequence of miR-30c. Examples of the mature sequence include those comprising a nucleic acid sequence of miRBase Accession No. MIMAT0000244 or MIMAT0004550. For example, the nucleic acid sequence of MIMAT0000244 is as follows: UGUAAACAUCCUACACUCUCAGC; and the nucleic acid sequence of MIMAT0004550 is as follows: CUGGGAGAAGGCUGUUUACUCU.

Detection of miR-30c encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-30c can involve detection using one or more probes and/or primers.

miR-3161

The anti-VEGF therapy resistance diagnostic miRNA miR-3161 is also referred to as “hsa-mir-3161”. Examples of the stem-loop sequence for hsa-mir-3161 include those comprising a nucleic acid sequence of miRBase Accession No. MI0014191 or naturally occurring variants thereof. For example, the nucleic acid stem-loop sequence of MI0014191 is as follows:

CCUCGAGAGCUGAUAAGAACAGAGGCCCAGAUUGAAGUUGAAUAGUGCUG
GGCCUUUGUUUUUACCAAGUUCCCUGG.

The stem-loop sequence may be processed to the mature sequence of miR-3161. Examples of the mature sequence include those comprising a nucleic acid sequence of miRBase Accession No. MIMAT0015035. For example, the nucleic acid sequence of MIMAT0015035 is as follows: CUGAUAAGAACAGAGGCCCAGAU.

Detection of miR-3161 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-3161 can involve detection using one or more probes and/or primers.

miR-3197

The anti-VEGF therapy resistance diagnostic miRNA miR-3197 is also referred to as “hsa-mir-3197”. Examples of the stem-loop sequence for hsa-mir-3197 include those comprising a nucleic acid sequence of miRBase Accession No. MI0014245 or naturally occurring variants thereof. For example, the nucleic acid stem-loop sequence of MI0014245 is as follows:

GGCGAGGGGAGGCGCAGGCUCGGAAAGGCGCGCGAGGCUCCAGGCUCCUU
CCCGAUCCACCGCUCUCCUCGCU.

The stem-loop sequence may be processed to the mature sequence of miR-3197. Examples of the mature sequence include those comprising a nucleic acid sequence of miRBase Accession No. MIMAT0015082. For example, the nucleic acid sequence of MIMAT0015082 is as follows: GGAGGCGCAGGCUCGGAAAGGCG.

Detection of miR-3197 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-3197 can involve detection using one or more probes and/or primers.

miR-483-3p

The anti-VEGF therapy resistance diagnostic miRNA miR-483-3p is also referred to as “hsa-mir-483-3p”. Examples of the stem-loop sequence for hsa-mir-483 include those comprising a nucleic acid sequence of miRBase Accession No. MI0002467 or naturally occurring variants thereof. For example, the nucleic acid stem-loop sequence of MI0002467 is as follows:

GAGGGGGAAGACGGGAGGAAAGAAGGGAGUGGUUCCAUCACGCCUCCUCA
CUCCUCUCCUCCCGUCUUCUCCUCUC.

The stem-loop sequence may be processed to the mature sequence of miR-483-3p. Examples of the mature sequence include those comprising a nucleic acid sequence of miRBase Accession No. MIMAT0002173. For example, the nucleic acid sequence of MIMAT0002173 is as follows: UCACUCCUCUCCUCCCGUCUU.

Detection of miR-483-3p encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-483-3p can involve detection using one or more probes and/or primers.

miR-550-star

The anti-VEGF therapy resistance diagnostic miRNA miR-550-star is also referred to as “hsa-mir-550-star”. Examples of the stem-loop sequence for hsa-mir-550 include those comprising a nucleic acid sequence of miRBase Accession No. MI0003600, MI0003601, MI0003762, or naturally occurring variants thereof. For example, the nucleic acid stem-loop sequence of MI0003600 is as follows: UGAUGCUUUGCUGGCUGGUGCAGUGCCUGAGGGAGUAAGAGCCCUGUU GUUGUAAGAUAGUGUCUUACUCCCUCAGGCACAUCUCCAACAAGUCUC U, the nucleic acid stem-loop sequence of MI0003601 is as follows: UGAUGCUUUGCUGGCUGGUGCAGUGCCUGAGGGAGUAAGAGCCCUGUU GUUGUCAGAUAGUGUCUUACUCCCUCAGGCACAUCUCCAGCGAGUCUCU, and the nucleic acid stem-loop sequence of MI0003762 is as follows:

GAUGCUUUGCUGGCUGGUGCAGUGCCUGAGGGAGUAAGAGUCCUGUUGUU
GUAAGAUAGUGUCUUACUCCCUCAGGCACAUCUCCAACAAGUCUC.

The stem-loop sequence may be processed to the mature sequence of miR-550-star. Examples of the mature sequence include those comprising a nucleic acid sequence of miRBase Accession No. MIMAT0003257. For example, the nucleic acid sequence of MIMAT0003257 is as follows:

UGUCUUACUCCCUCAGGCACAU.

Detection of miR-550-star encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-550-star can involve detection using one or more probes and/or primers.

miR-573

The anti-VEGF therapy resistance diagnostic miRNA miR-573 is also referred to as “hsa-miR-573”. Examples of the stem-loop sequence for hsa-mir-573 include those comprising a nucleic acid sequence of miRBase Accession No. MI0003580 or naturally occurring variants thereof. For example, the nucleic acid stem-loop sequence of MI0003580 is as follows:

UUUAGCGGUUUCUCCCUGAAGUGAUGUGUAACUGAUCAGGAUCUACUCAU
GUCGUCUUUGGUAAAGUUAUGUCGCUUGUCAGGGUGAGGAGAGUUUUUG.

The stem-loop sequence may be processed to the mature sequence of miR-573. Examples of the mature sequence include those comprising a nucleic acid sequence of miRBase Accession No. MIMAT0003238. For example, the nucleic acid sequence of MIMAT0003238 is as follows:

CUGAAGUGAUGUGUAACUGAUCAG.

Detection of miR-573 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-573 can involve detection using one or more probes and/or primers.

miR-577

The anti-VEGF therapy resistance diagnostic miRNA miR-577 is also referred to as “hsa-miR-577”. Examples of the stem-loop sequence for hsa-mir-577 include those comprising a nucleic acid sequence of miRBase Accession No. MI0003584 or naturally occurring variants thereof. For example, the nucleic acid stem-loop sequence of MI0003584 is as follows:

UGGGGGAGUGAAGAGUAGAUAAAAUAUUGGUACCUGAUGAAUCUGAGGCC
AGGUUUCAAUACUUUAUCUGCUCUUCAUUUCCCCAUAUCUACUUAC.

The stem-loop sequence may be processed to the mature sequence of miR-577. Examples of the mature sequence include those comprising a nucleic acid sequence of miRBase Accession No. MIMAT0003242. For example, the nucleic acid sequence of MIMAT0003242 is as follows: UAGAUAAAAUAUUGGUACCUG.

Detection of miR-577 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-577 can involve detection using one or more probes and/or primers.

miR-let-7b

The anti-VEGF therapy resistance diagnostic miRNA miR-let-7b, which may also be referred to as “hsa-let-7b” is a member of the let-7 gene family. Examples of the stem-loop sequence for hsa-let-7b include those comprising a nucleic acid sequence of miRBase Accession No. MI0000063 or naturally occurring variants thereof. For example, the nucleic acid stem-loop sequence of MI0000063 is as follows:

CGGGGUGAGGUAGUAGGUUGUGUGGUUUCAGGGCAGUGAUGUUGCCCCUC
GGAAGAUAACUAUACAACCUACUGCCUUCCCUG.

The stem-loop sequence may be processed to the mature sequence of miR-let-7b. Examples of the mature sequence include those comprising a nucleic acid sequence of miRBase Accession No. MIMAT0000063 or MIMAT0004482. For example, the nucleic acid sequence of MIMAT0000063 is as follows: UGAGGUAGUAGGUUGUGUGGUU; and the nucleic acid sequence of MIMAT0004482 is as follows: CUAUACAACCUACUGCCUUCCC.

Detection of miR-let-7b encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-let-7b can involve detection using one or more probes and/or primers.

miR-486

The anti-VEGF therapy resistance diagnostic miRNA miR-486, which may also be referred to as “hsa-miR-486” is a member of the miR-486 gene family. Examples of the stem-loop sequence for hsa-mir-486 include those comprising a nucleic acid sequence of miRBase Accession No. MI0002470 or naturally occurring variants thereof. For example, the nucleic acid stem-loop sequence of MI0002470 is as follows:

GCAUCCUGUACUGAGCUGCCCCGAGGCCCUUCAUGCUGCCCAGCUCGGGG
CAGCUCAGUACAGGAUAC.

The stem-loop sequence may be processed to the mature sequence of miR-486. Examples of the mature sequence include those comprising a nucleic acid sequence of miRBase Accession No. MIMAT0002177 or MIMAT0004762. For example, the nucleic acid sequence of MIMAT0002177 is as follows: UCCUGUACUGAGCUGCCCCGAG; and the nucleic acid sequence of MIMAT0004762 is as follows: CGGGGCAGCUCAGUACAGGAU.

Detection of miR-486 encompasses detection of the mature miRNA, as well as detection of naturally occurring variants and fragments thereof found in a biological sample, and detection of a precursor molecule of the aforementioned miRNAs, such as the corresponding pri-miRNAs or pre-miRNAs. Detection of miR-486 can involve detection using one or more probes and/or primers.

### Subjects

The methods of the present disclosure can be used to determine a likelihood of having or developing diabetic retinopathy (DR) in a subject, a likelihood of having or developing dry or wet age-related macular degeneration (AMD) in a subject, or a likelihood of having or developing a resistance to anti-VEGF therapy in a subject. The subject can be any subject having, suspected of having, or at risk of, DR or AMD. Subjects include patients undergoing therapy, e.g., undergoing therapy to treat suspected or diagnosed DR or AMD.

Subjects to be tested using a method of the present disclosure include individuals who present with or have presented with one or more symptoms of DR or AMD. Symptoms of DR include blurred, double, or distorted vision; difficulty reading; floaters or spots in one's vision; partial or total loss of vision or a shadow or veil across one's field of vision; pain, pressure, or constant redness of the eye. Symptoms of AMD include dim, fuzzy, or less sharp central vision; difficulty reading, difficulty seeing faces clearly; objects appear distorted or smaller than they actually are; a new blank or blind spot develops in the central field of vision; straight lines begin to appear wavy or curved.

### Biological Samples

Suitable biological samples useful in the methods of the present disclosure include biological fluids (e.g., a blood sample, e.g., whole blood, blood fraction (e.g., serum, plasma), vitreous humor, aqueous humor), and other liquid samples of biological origin, as well as solid tissue samples. Where the biological sample is a blood sample, the blood sample can be obtained from fresh blood or stored blood (e.g. in a blood bank). The biological sample can be a blood sample expressly obtained for an assay of the present disclosure or a blood sample obtained for another purpose which can be subsampled for an assay of the present disclosure. Where the biological sample is vitreous humor or aqueous humor, the sample can be obtained from fresh aqueous or vitreous humor or stored aqueous or vitreous humor. The biological sample can be a blood sample or sample of the aqueous or vitreous humor expressly obtained for an assay of the present disclosure or a sample obtained for another purpose which can be subsampled for an assay of the present disclosure.

Samples can be manipulated after procurement, such as by treatment with reagents, solubilization, and/or enrichment for certain components for an analyte(s) to be assayed. Samples can be pretreated as necessary by dilution in an appropriate buffer solution, concentrated if desired, or fractionated by any number of methods including but not limited to ultracentrifugation, fractionation by fast performance liquid chromatography (FPLC), or precipitation. For example, in certain aspects the sample is subfractionated into, e.g., vesicular and non-vesicular components (e.g. naked ribonucleoproteins) before subsequent analysis. Suitable means of sub-fractioning a sample are known in the art and described in, e.g., Duttagupta R, et al. (2011) PLoS ONE 6(6): e20769; the disclosure of which is incorporated herein by reference. Any of a number of standard aqueous buffer solutions, employing one of a variety of buffers, such as phosphate, Tris, or the like, at physiological pH can be used. In general, after isolation, samples (such as blood samples) are stored at −80° C. until assaying.

### Assay Formats and Detection Methods

Biomarkers for analysis in connection with the methods of the present disclosure can be detected using a variety of methods, with methods suitable for quantitative and semi-quantitative assays being of particular interest. Examples of detection methods include, but are not limited to, various assays involving reverse transcription of RNA and nucleic acid amplification (e.g., PCR, quantitative real time PCR, nucleic acid microarrays, sequencing, bead arrays, and the like).

For example, isolated miRNA from a biological sample can be used in hybridization or amplification assays that include, but are not limited to, Southern or Northern analyses, polymerase chain reaction (PCR) analyses and probe arrays. One method for the detection of miRNA levels involves contacting the isolated miRNA with a nucleic acid molecule (probe) that can hybridize to biomarker-encoding nucleic acid. The nucleic acid probe can be for example, a full-length cDNA, or a portion thereof, such as an oligonucleotide of at least 7, 15, 30, 50, 100, 250 or 500 nucleotides in length and sufficient to specifically hybridize under stringent conditions to biomarker-encoding nucleic acid.

In one embodiment, the miRNA from a biological sample is immobilized on a solid surface and contacted with a probe. In an alternative embodiment, the probe(s) are immobilized on a solid surface and the miRNA isolated from the biological sample is contacted with the probe(s), e.g., as in an array format.

Methods of detecting levels of biomarker expression in a sample can involve any suitable method of nucleic acid amplification, e.g., by RT-PCR, ligase chain reaction, or any other nucleic acid amplification method, followed by the detection of the amplified molecules using techniques well known to those of skill in the art. In one example, biomarker expression is assessed by quantitative fluorogenic RT-PCR (e.g., such as using TaqMan™, SYBR Green, and the like). Such methods typically utilize pairs of oligonucleotide primers that are specific for a biomarker-encoding nucleic acid. Methods for designing oligonucleotide primers specific for a known sequence are well known in the art.

In certain embodiments, the method employs a detection approach that involves multiplex qRT-PCR, such as the microfluidic-based multiplex qRT-PCR method as described in Moltzahn, et al. (2010) Cancer Res 71:550-560; the disclosure of which is incorporated herein by reference. Detection methods of interest further include, but are not limited to, those described in Mitchell P S, et al. (2008) PNAS 105(30):10513-10518 and U.S. Patent Publication Nos. 2011/0275534 and 2012/0264638; the disclosures of which are incorporated herein by reference.

### Determination of a Likelihood of Having or Developing DR, AMD or Resistance to Anti-VEGF Treatment

The methods of the present disclosure include methods for determining a likelihood of having or developing DR, AMD or resistance to anti-VEGF therapy in a subject. Such methods generally involve determining an amount of at least one diagnostic miRNA (i.e., using an assay or detection method as described herein). The amount of the diagnostic miRNA(s) may be compared with a control amount, and the results of the comparison may be used to indicate the likelihood of having or developing DR, AMD or resistance to anti-VEGF therapy in the subject.

The control amount of the diagnostic miRNA may be the amount of the diagnostic miRNA in a healthy individual or a population of healthy individuals. The control amount may, in some aspects, be the amount of the diagnostic miRNA from a different individual or group of individuals, such as a control population that does not have a resistance to anti-VEGF therapy.

In some embodiments, the control amount is an amount of the diagnostic miRNA from the same subject, measured at a prior time point. For instance, the prior time point may be a time point that is prior to the subject exhibiting clinical presentations of DR or AMD and/or at an earlier stage of the disease.

In addition to the amount of one or more diagnostic miRNA(s), methods of the present disclosure may involve the use of one or more additional risk factors to determine a likelihood of having or developing DR or AMD. Risk factors for DR include poor glucose control, high blood pressure, nephropathy, pregnancy, smoking, obesity, and elevated cholesterol levels. Risk factors for AMD include age (over 50 years old), family history of AMD, race, smoking, obesity, diet, cardiovascular disease and elevated cholesterol levels.

The amounts of diagnostic miRNA(s) and/or one or more additional risk factors may be combined to provide an assessment of the likelihood of having or developing DR or AMD in a subject, or having or developing a resistance to anti-VEGF therapy.

### Reports

The methods of the present disclosure can include generating a report indicating the results of the method and providing guidance as to how the results might be applied to the care of the subject. A “report,” as described herein, refers generally to an electronic document or file (e.g., pdf file, monitor display), as well as a tangible document (e.g., paper report). A subject report can be completely or partially electronically generated, e.g., presented on an electronic display (e.g., computer monitor).

The method results in the report can include, for example, one or more of the amount of the diagnostic miRNA assayed. The level can be reported as a quantitative score (e.g., a concentration, e.g., pg/ml serum) and/or a semi-quantitative score (e.g., a score reflecting an amount of a biomarker relative to a control level or a selected threshold level). The method results can optionally include assay results for a control biomarker.

Reports can include information such as a predicted risk that the patient has or will develop DR, AMD or a resistance to anti-VEGF therapy.

Reports can include guidance to a clinician as to a treatment recommendation for the subject based on the likelihood of having or developing DR, AMD or a resistance to anti-VEGF therapy in a subject. For example, reports can include a recommendation regarding further evaluation and/or avoiding expensive and invasive evaluations and/or a recommendations regarding therapeutic intervention (e.g., administering a drug, recommending surgical intervention, etc.), modifying a treatment regimen (e.g., adjusting a drug dose (e.g., increasing or decreasing a dose), adjusting a dosage regimen (e.g., increasing or decreasing dose frequency and/or amount), and the like.

A report can further include one or more of: 1) patient information (e.g., name, medical information (e.g., age, gender, symptoms (e.g., symptoms that may be relevant to diagnosis DR or AMD), etc.), 2) information about the biological sample (e.g., type, when obtained); 3) information regarding where and how the assay was performed (e.g., testing facility, assay format); 4) service provider information; and/or 5) an interpretive report, which can provide a narrative providing an at least partial interpretation of the results so as to facilitate a diagnosis by a clinician.

Accordingly, the methods disclosed herein can further include a step of generating or outputting a report providing the method results and, optionally, other information such as treatment guidance as described herein. The report can be provided in the form of an electronic medium (e.g., an electronic display on a computer monitor), or in the form of a tangible medium (e.g., a report printed on paper or other tangible medium). An assessment as to the likelihood can be referred to as “risk report” or, simply, a “diagnostic result”. The person or entity that prepares a report (“report generator”) may also perform steps such as sample gathering, sample processing, and the like. Alternatively, an entity other than the report generator can perform steps such as sample gathering, sample processing, and the like. A report can be provided to a user. A “user” can be, for example, a health professional (e.g., a clinician, a laboratory technician, a physician, etc.).

### Computer-Implemented Methods, Systems and Devices

The methods of the present disclosure can be computer-implemented, such that method steps (e.g., assaying, comparing, calculating, and the like) are automated in whole or in part. Accordingly, the present disclosure provides methods, computer systems, devices and the like in connection with computer-implemented methods of determining a likelihood of having or developing DR, AMD or a resistance to anti-VEGF therapy in a subject.

For example, the method steps, including obtaining values for the diagnostic miRNA(s), comparing diagnostic miRNA amount(s) to a control amount, generating a report, and the like, can be completely or partially performed by a computer program product. Values obtained can be stored electronically, e.g., in a database, and can be subjected to an algorithm executed by a programmed computer.

For example, the methods of the present disclosure can involve inputting the amount of a diagnostic miRNA into a computer programmed to execute an algorithm to perform the comparing step described herein, and generate a report as described herein, e.g., by displaying or printing a report to an output device at a location local or remote to the computer.

The present invention thus provides a computer program product including a computer readable storage medium having a computer program stored on it. The program can, when read by a computer, execute relevant calculations based on values obtained from analysis of one or more biological sample from an individual. The computer program product has stored therein a computer program for performing the calculation(s).

The present disclosure provides systems for executing the program described above, which system generally includes: a) a central computing environment; b) an input device, operatively connected to the computing environment, to receive patient data, wherein the patient data can include, for example, biomarker level or other value obtained from an assay using a biological sample from the patient, as described above; c) an output device, connected to the computing environment, to provide information to a user (e.g., medical personnel); and d) an algorithm executed by the central computing environment (e.g., a processor), where the algorithm is executed based on the data received by the input device, and wherein the algorithm calculates a value, which value is indicative of the likelihood of the subject having or developing DR, AMD, or a resistance to anti-VEGF therapy, as described herein.

### Computer Systems

The present disclosure also provides computer systems for determining a likelihood of having or developing DR, AMD, or resistance to anti-VEGF therapy in a subject. The computer systems include a processor and memory operably coupled to the processor, where the memory programs the processor to receive assay data including an amount of at least one diagnostic miRNA in a biological sample from a subject; compare the amount received with a control amount; and calculate a likelihood of having or developing DR, AMD, or resistance to anti-VEGF therapy in the subject, based on results of said comparing the determined amounts obtained from the analyzing unit with the control amount. In certain aspects, the system calculates a likelihood of having or developing DR, AMD, or resistance to anti-VEGF therapy in the subject based on one or more additional risk factors, such as the subject's age, etc.

Computer systems may include a processing system, which generally comprises at least one processor or processing unit or plurality of processors, memory, at least one input device and at least one output device, coupled together via a bus or group of buses. In certain embodiments, an input device and output device can be the same device. The memory can be any form of memory device, for example, volatile or non-volatile memory, solid state storage devices, magnetic devices, etc. The processor can comprise more than one distinct processing device, for example to handle different functions within the processing system.

An input device receives input data and can comprise, for example, a keyboard, a pointer device such as a pen-like device or a mouse, audio receiving device for voice controlled activation such as a microphone, data receiver or antenna such as a modem or wireless data adaptor, data acquisition card, etc. Input data can come from different sources, for example keyboard instructions in conjunction with data received via a network.

Output devices produce or generate output data and can comprise, for example, a display device or monitor in which case output data is visual, a printer in which case output data is printed, a port for example a USB port, a peripheral component adaptor, a data transmitter or antenna such as a modem or wireless network adaptor, etc. Output data can be distinct and derived from different output devices, for example a visual display on a monitor in conjunction with data transmitted to a network. A user can view data output, or an interpretation of the data output, on, for example, a monitor or using a printer. The storage device can be any form of data or information storage means, for example, volatile or non-volatile memory, solid state storage devices, magnetic devices, etc.

In use, the processing system may be adapted to allow data or information to be stored in and/or retrieved from, via wired or wireless communication means, at least one database. The interface may allow wired and/or wireless communication between the processing unit and peripheral components that may serve a specialized purpose. In general, the processor can receive instructions as input data via input device and can display processed results or other output to a user by utilizing output device. More than one input device and/or output device can be provided. A processing system may be any suitable form of terminal, server, specialized hardware, or the like.

A processing system may be a part of a networked communications system. A processing system can connect to a network, for example the Internet or a WAN. Input data and output data can be communicated to other devices via the network. The transfer of information and/or data over the network can be achieved using wired communications means or wireless communications means. A server can facilitate the transfer of data between the network and one or more databases. A server and one or more databases provide an example of an information source.

Thus, a processing computing system environment may operate in a networked environment using logical connections to one or more remote computers. The remote computer may be a personal computer, a server, a router, a network PC, a peer device, or other common network node, and typically includes many or all of the elements described above.

Certain embodiments may be described with reference to acts and symbolic representations of operations that are performed by one or more computing devices. As such, it will be understood that such acts and operations, which are at times referred to as being computer-executed, include the manipulation by the processor of the computer of electrical signals representing data in a structured form. This manipulation transforms the data or maintains them at locations in the memory system of the computer, which reconfigures or otherwise alters the operation of the computer in a manner understood by those skilled in the art. The data structures in which data is maintained are physical locations of the memory that have particular properties defined by the format of the data. However, while an embodiment is being described in the foregoing context, it is not meant to be limiting as those of skill in the art will appreciate that the acts and operations described hereinafter may also be implemented in hardware.

Embodiments may be implemented with numerous other general-purpose or special-purpose computing devices and computing system environments or configurations. Examples of well-known computing systems, environments, and configurations that may be suitable for use with an embodiment include, but are not limited to, personal computers, handheld or laptop devices, personal digital assistants, multiprocessor systems, microprocessor-based systems, programmable consumer electronics, network, minicomputers, server computers, web server computers, mainframe computers, and distributed computing environments that include any of the above systems or devices.

Embodiments may be described in a general context of computer-executable instructions, such as program modules, being executed by a computer. Generally, program modules include routines, programs, objects, components, data structures, etc., that perform particular tasks or implement particular abstract data types. An embodiment may also be practiced in a distributed computing environment where tasks are performed by remote processing devices that are linked through a communications network. In a distributed computing environment, program modules may be located in both local and remote computer storage media including memory storage devices.

### Computer Program Products

The present disclosure provides computer program products that, when executed on a programmable computer such as that described above, can carry out the methods of the present disclosure. As discussed above, the subject matter described herein may be embodied in systems, apparatus, methods, and/or articles depending on the desired configuration. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device (e.g. video camera, microphone, joystick, keyboard, and/or mouse), and at least one output device (e.g. display monitor, printer, etc.).

Computer programs (also known as programs, software, software applications, applications, components, or code) include instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, etc.) used to provide machine instructions and/or data to a programmable processor, including a machine-readable medium that receives machine instructions as a machine-readable signal.

It will be apparent from this description that aspects of the present invention may be embodied, at least in part, in software, hardware, firmware, or any combination thereof. Thus, the techniques described herein are not limited to any specific combination of hardware circuitry and/or software, or to any particular source for the instructions executed by a computer or other data processing system. Rather, these techniques may be carried out in a computer system or other data processing system in response to one or more processors, such as a microprocessor, executing sequences of instructions stored in memory or other computer-readable medium including any type of ROM, RAM, cache memory, network memory, floppy disks, hard drive disk (HDD), solid-state devices (SSD), optical disk, CD-ROM, and magnetic-optical disk, EPROMs, EEPROMs, flash memory, or any other type of media suitable for storing instructions in electronic format.

In addition, the processor(s) may be, or may include, one or more programmable general-purpose or special-purpose microprocessors, digital signal processors (DSPs), programmable controllers, application specific integrated circuits (ASICs), programmable logic devices (PLDs), trusted platform modules (TPMs), or the like, or a combination of such devices. In alternative embodiments, special-purpose hardware such as logic circuits or other hardwired circuitry may be used in combination with software instructions to implement the techniques described herein.

### Examples of Applications of Method Results

The methods of the present disclosure can provide results which can then be applied to facilitate decisions as to the care of the subject. Examples are provided below.

### Assay-Guided Therapy and Monitoring of Therapy

The methods of the present disclosure can help a clinician in making a treatment decision for the subject, e.g., where the results of the method suggest the subject may or may not benefit from invasive therapeutic intervention for treatment of DR or AMD. For example, based on the method results, a therapy can be selected for the subject based on the likelihood he has or is at risk of having DR, AMD or a resistance to anti-VEGF therapy. Clinical signs, symptoms and other factors such as family history can also be considered to facilitate selecting a therapy.

The method results can guide a clinician as to whether or not any therapy for treatment of DR or AMD should be administered, e.g., administration of an anti-VEGF therapy.

The methods of the present disclosure can facilitate monitoring therapy of a subject undergoing treatment for DR or AMD. For example, where the subject is already receiving a therapy, the method can provide a method of monitoring therapy. In this case, the method results can guide a clinician in adjusting therapy (e.g., whether or not to continue therapy (e.g., so as to avoid relapse), increase or decrease dose, change therapy regimen (e.g., from monotherapy to combination therapy, or from non-surgical therapy to surgical therapy) where the patient is not receiving adequate therapeutic benefit (e.g., the patient is not responding to therapy), and the like). Such methods of monitoring therapy are useful in guiding further treatment decisions, such as whether continued administration of a drug regimen is indicated, or whether the patient should receive a surgical procedure. The methods of monitoring therapy of the present disclosure may be used in combination with other methods for assessing whether a subject responds to therapy (is a “responder”) or is not exhibiting a sufficient therapeutically beneficial response (is as “non-responder”).

### Identifying Subjects for Clinical Trial Populations

The methods of the present disclosure find use in identifying subjects suitable for inclusion or exclusion in a clinical trial based on the likelihood of having or developing DR or AMD. For example, the methods of the present disclosure can be used to identify subjects suitable for inclusion in a clinical trial. In another example, the methods of the present disclosure can be used to identify subjects with a likelihood of having or developing DR or AMD so as to exclude such subjects from a clinical trial. Accordingly, such methods can facilitate identification of drugs or other therapies for treatment of DR or AMD.

### Kits

Kits of the present disclosure can include a detection agent(s) for one or more, two or more, three or more, four or more, five or more, six or more, seven or more, eight or more, nine or more, or 10 or more diagnostic miRNAs. As used herein, a “detection reagent” refers to a binding partner for a biomarker that is suitable for use in detection of a biomarker, and is optionally detectably labeled. Detection agent(s) for one or more control miRNA(s) can also be included. Kits can include one or more devices, computer systems, devices and the like, including such devices and computer systems as described herein.

Kits can include instructions for using the components of the kit to practice a method of the present disclosure. The instructions are generally recorded on a suitable recording medium, such as paper, plastic, electronic storage, medium, and the like. For example, the instructions may be present in the kits as a package insert, in the labeling of the container of the kit or components thereof (e.g., associated with the packaging or sub-packaging), etc. In other embodiments, the instructions are present as an electronic storage data file present on a suitable computer readable storage medium, e.g. compact disc-read only memory (CD-ROM), digital versatile disk (DVD), diskette, etc. In other examples, the instructions provided do not contain many or all assay details, but rather provide direction as to a remote source for obtaining detailed instructions, e.g. via the internet.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-106 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of determining a likelihood of having or developing diabetic retinopathy (DR) in a subject, the method comprising:

determining an amount of at least one DR diagnostic miRNA in a biological sample from the subject;

comparing the amount of the at least one DR diagnostic miRNA with a control amount of the at least one DR diagnostic miRNA; and

generating a report indicating a likelihood of having or developing DR in the subject based on results of said comparing the amount of the at least one DR diagnostic miRNA with the control amount of the at least one DR diagnostic miRNA.
2. The method according to claim 1, wherein the biological sample is aqueous humor or vitreous humor.
3. The method according to 2, wherein the biological sample is aqueous humor and the DR diagnostic miRNA is miR-let-7b and/or miR-let-7d.
4. The method according to 2, wherein the biological sample is vitreous humor and the DR diagnostic miRNA is miR-let-7a, miR-let-7b, miR-let-7c and/or miR-let-7d.
5. The method according to 2, wherein the biological sample is aqueous humor and the DR diagnostic miRNA is miR-320c.
6. The method according to 2, wherein the biological sample is vitreous humor and the DR diagnostic miRNA is miR-320a, miR-320b and/or miR-320c.
7. The method according to 2, wherein the biological sample is aqueous humor and the DR diagnostic miRNA is miR-26a.
8. The method according to 2, wherein the biological sample is aqueous humor and the DR diagnostic miRNA is miR-4488.
9. The method according to 2, wherein the biological sample is vitreous humor and the DR diagnostic miRNA is miR-4488.
10. The method according to 2, wherein the biological sample is aqueous humor and the DR diagnostic miRNA is miR-638.
11. The method according to 2, wherein the biological sample is aqueous humor and the DR diagnostic miRNA is miR-29a.
12. The method according to 2, wherein the biological sample is vitreous humor and the DR diagnostic miRNA is miR-29a.
13. The method according to any one of 1-12, comprising obtaining the biological sample from the subject.
14. The method according to any one of 1-13, wherein determining the amount of the at least one DR diagnostic miRNA comprises performing quantitative real-time PCR.
15. The method according to 14, wherein the quantitative real-time PCR is multiplexed.
16. The method according to any one of 1-15, wherein the report comprises guidance to a clinician as to a treatment recommendation for the subject based on the likelihood of having or developing DR in the subject.
17. The method according to 16, wherein the treatment recommendation is control of blood glucose, blood pressure and/or blood cholesterol.
18. The method according to 16, wherein the treatment recommendation is administration of an anti-VEGF antibody.
19. The method according to 16, wherein the treatment recommendation is scatter laser treatment.
20. The method according to 16, wherein the treatment recommendation is focal laser treatment.
21. The method according to any one of 1-20, comprising inputting the amount of the at least one DR diagnostic miRNA into a computer comprising a processor programmed to perform the comparing step, wherein said inputting generates a result for a report.
22. The method according to 21, wherein said generating a report is performed by the computer.
23. The method according to 21 or 22, wherein the report is displayed to an output device at a location remote to the computer.
24. A method of determining efficacy of a therapy for treatment of diabetic retinopathy (DR) in a subject, the method comprising:

determining an amount of at least one DR diagnostic miRNA in a biological sample from the subject prior to administration of the therapy;

administering the therapy;

determining an amount of the least one DR diagnostic miRNA in a biological sample from the subject following administration of the therapy;

comparing the amount of the at least one DR diagnostic miRNA in a biological sample prior to administration of the therapy with the amount of the at least one DR diagnostic miRNA in a biological sample following administration of the therapy; and generating a report indicating efficacy of the therapy based on results of said comparing.
25. The method according to 24, wherein the determining an amount of at least one DR diagnostic miRNA in a biological sample from the subject prior to administration of the therapy comprises determining an amount of at least one DR diagnostic miRNA in a biological sample from the subject prior to administration of the therapy relative to a control amount of the at least one DR diagnostic miRNA.
26. The method according to 24 or 25, wherein the determining an amount of at least one DR diagnostic miRNA in a biological sample from the subject following administration of the therapy comprises determining an amount of at least one DR diagnostic miRNA in a biological sample from the subject following administration of the therapy relative to a control amount of the at least one DR diagnostic miRNA.
27. The method according to any one of 24-26, wherein the biological sample is aqueous humor or vitreous humor.
28. The method according to 27, wherein the biological sample is aqueous humor and the DR diagnostic miRNA is miR-let-7b and/or miR-let-7d.
29. The method according to 27, wherein the biological sample is vitreous humor and the DR diagnostic miRNA is miR-let-7a, miR-let-7b, miR-let-7c and/or miR-let-7d.
30. The method according to 27, wherein the biological sample is aqueous humor and the DR diagnostic miRNA is miR-320c.
31. The method according to 27, wherein the biological sample is vitreous humor and the DR diagnostic miRNA is miR-320a, miR-320b and/or miR-320c.
32. The method according to 27, wherein the biological sample is aqueous humor and the DR diagnostic miRNA is miR-26a.
33. The method according to 27, wherein the biological sample is aqueous humor and the DR diagnostic miRNA is miR-4488.
34. The method according to 27, wherein the biological sample is vitreous humor and the DR diagnostic miRNA is miR-4488.
35. The method according to 27, wherein the biological sample is aqueous humor and the DR diagnostic miRNA is miR-638.
36. The method according to 27, wherein the biological sample is aqueous humor and the DR diagnostic miRNA is miR-29a.
37. The method according to 27, wherein the biological sample is vitreous humor and the DR diagnostic miRNA is miR-29a.
38. The method according to any one of 24-37, comprising obtaining the biological sample from the subject.
39. The method according to any one of 24-38, wherein determining the amount of the at least one DR diagnostic miRNA comprises performing quantitative real-time PCR.
40. The method according to 39, wherein the quantitative real-time PCR is multiplexed.
41. The method according to any one of 24-40, wherein the report comprises guidance to a clinician as to a treatment recommendation for the subject based on the efficacy of therapy.
42. The method according to 41, wherein the treatment recommendation is continued administration of the therapy.
43. The method according to 41, wherein the treatment recommendation is termination of administration of the therapy.
44. The method according to 41, wherein the treatment recommendation is a dosage adjustment in connection with the therapy.
45. The method according to any one of 24-44, comprising inputting the amount of the at least one DR diagnostic miRNA in a biological sample from the subject prior to administration of the therapy and the amount of the least one DR diagnostic miRNA in a biological sample from the subject following administration of the therapy into a computer comprising a processor programmed to perform the comparing step, wherein said inputting generates a result for a report.
46. The method according to 45, wherein said generating a report is performed by the computer.
47. The method according to 45 or 46, wherein the report is displayed to an output device at a location remote to the computer.
48. A device for determining a likelihood of having or developing diabetic retinopathy (DR) in a subject, the device comprising:

an analyzing unit comprising a detection agent for at least one DR diagnostic miRNA molecule, wherein the analyzing unit determines an amount of the at least one DR diagnostic miRNA molecule in a biological sample detected by the detection agent; and

an evaluation unit comprising a processor programmed to:
compare the determined amount obtained from the analyzing unit with a control amount of the at least one DR diagnostic miRNA; and
calculate a likelihood of having or developing DR in a subject, based on results of said comparing the determined amount obtained from the analyzing unit with the control amount.
49. The device according to 48, comprising a display comprising a user interface, the display in electronic communication with the processor of the evaluation unit.
50. A computer system, the system comprising:

a processor; and

memory operably coupled to the processor, wherein the memory programs the processor to:
receive assay data including a determined amount of at least one diabetic retinopathy (DR) diagnostic miRNA in a biological sample from a subject;
compare the amount received with a control amount of the at least one DR diagnostic miRNA; and
calculate a likelihood of having or developing DR in a subject, based on results of said comparing the determined amounts with the control amount.
51. The system according 50, comprising a display comprising a user interface, the display in electronic communication with the processor.
52. A kit for determining a likelihood of having or developing diabetic retinopathy (DR) in a subject, the kit comprising:

a detection agent for at least one DR diagnostic miRNA molecule; and

a device according to 48 or 49.
53. A method of determining a likelihood of having or developing dry or wet age-related macular degeneration (AMD) in a subject, the method comprising:

determining an amount of at least one dry or wet AMD diagnostic miRNA in a biological sample from the subject;

comparing the amount of the at least one dry or wet AMD diagnostic miRNA with a control amount of the at least one dry or wet AMD diagnostic miRNA; and

generating a report indicating a likelihood of having or developing dry or wet AMD in the subject based on results of said comparing the amount of the at least one dry or wet AMD diagnostic miRNA with the control amount of the at least one dry or wet AMD diagnostic miRNA.
54. The method according to 53, wherein the biological sample is serum.
55. The method according to 53 or 54, wherein the at least one dry or wet AMD diagnostic miRNA is at least one wet AMD diagnostic miRNA.
56. The method according to 55, wherein the at least one wet AMD diagnostic miRNA is selected from the group consisting of: miR-10b, miR-1306, miR-136, miR-183-star, miR-296-5p, miR-30c, miR-4258, miR-519d, miR-889, and combinations thereof.
57. The method according to 53 or 54, wherein the at least one dry or wet AMD diagnostic miRNA is at least one dry AMD diagnostic miRNA.
58. The method according to 57, wherein the at least one dry AMD diagnostic miRNA is selected from the group consisting of: miR-1224-3p, miR-1226, miR-29c, miR-3121, miR-3197, miR-32, miR-363-star, miR-639, miR-661, miR-708-star, and combinations thereof
59. The method according to any one of 53-58, comprising obtaining the biological sample from the subject.
60. The method according to any one of 53-59, wherein determining the amount of the at least one dry or wet AMD diagnostic miRNA comprises performing quantitative real-time PCR.
61. The method according to 60, wherein the quantitative real-time PCR is multiplexed.
62. The method according to any one of 53-61, wherein the report comprises guidance to a clinician as to a treatment recommendation for the subject based on the likelihood of having or developing dry or wet AMD in the subject.
63. The method according to 62, wherein the treatment recommendation is administration of an anti-VEGF antibody.
64. The method according to any one of 53-63, comprising inputting the amount of the at least one dry or wet AMD diagnostic miRNA into a computer comprising a processor programmed to perform the comparing step, wherein said inputting generates a result for a report.
65. The method according to 64, wherein said generating a report is performed by the computer.
66. The method according to 64 or 65, wherein the report is displayed to an output device at a location remote to the computer.
67. A method of determining efficacy of a therapy for treatment of dry or wet age-related macular degeneration (AMD) in a subject, the method comprising:

determining an amount of at least one dry or wet AMD diagnostic miRNA in a biological sample from the subject prior to administration of the therapy;

administering the therapy;

determining an amount of the least one dry or wet AMD diagnostic miRNA in a biological sample from the subject following administration of the therapy;

comparing the amount of the at least one dry or wet AMD diagnostic miRNA in a biological sample prior to administration of the therapy with the amount of the at least one dry or wet AMD diagnostic miRNA in a biological sample following administration of the therapy; and

generating a report indicating efficacy of the therapy based on results of said comparing.
68. The method according to 67, wherein the determining an amount of at least one dry or wet AMD diagnostic miRNA in a biological sample from the subject prior to administration of the therapy comprises determining an amount of at least one dry or wet AMD diagnostic miRNA in a biological sample from the subject prior to administration of the therapy relative to a control amount of the at least one dry or wet AMD diagnostic miRNA.
69. The method according to 67 or 68, wherein the determining an amount of at least one dry or wet AMD diagnostic miRNA in a biological sample from the subject following administration of the therapy comprises determining an amount of at least one dry or wet AMD diagnostic miRNA in a biological sample from the subject following administration of the therapy relative to a control amount of the at least one dry or wet AMD diagnostic miRNA.
70. The method according to any one of 67-69, wherein the biological sample is serum.
71. The method according to any one of 67-70, wherein the at least one dry or wet AMD diagnostic miRNA is at least one wet AMD diagnostic miRNA.
72. The method according to 71, wherein the at least one wet AMD diagnostic miRNA is selected from the group consisting of: miR-10b, miR-1306, miR-136, miR-183-star, miR-296-5p, miR-30c, miR-4258, miR-519d, miR-889, and combinations thereof.
73. The method according to any one of 67-70, wherein the at least one dry or wet AMD diagnostic miRNA is at least one dry AMD diagnostic miRNA.
74. The method according to 73, wherein the at least one dry AMD diagnostic miRNA is selected from the group consisting of: miR-1224-3p, miR-1226, miR-29c, miR-3121, miR-3197, miR-32, miR-363-star, miR-639, miR-661, miR-708-star, and combinations thereof
75. The method according to any one of 67-74, comprising obtaining the biological sample from the subject.
76. The method according to any one of 67-75, wherein determining the amount of the at least one dry or wet AMD diagnostic miRNA comprises performing quantitative real-time PCR.
77. The method according to 76, wherein the quantitative real-time PCR is multiplexed.
78. The method according to any one of 67-77, wherein the report comprises guidance to a clinician as to a treatment recommendation for the subject based on the efficacy of therapy.
79. The method according to 78, wherein the treatment recommendation is continued administration of the therapy.
80. The method according to 78, wherein the treatment recommendation is termination of administration of the therapy.
81. The method according to 78, wherein the treatment recommendation is a dosage adjustment in connection with the therapy.
82. The method according to any one of 67-81, comprising inputting the amount of the at least one dry or wet AMD diagnostic miRNA in a biological sample from the subject prior to administration of the therapy and the amount of the least one dry or wet AMD diagnostic miRNA in a biological sample from the subject following administration of the therapy into a computer comprising a processor programmed to perform the comparing step, wherein said inputting generates a result for a report.
83. The method according to 82, wherein said generating a report is performed by the computer.
84. The method according to 82 or 83, wherein the report is displayed to an output device at a location remote to the computer.
85. A device for determining a likelihood of having or developing dry or wet age-related macular degeneration (AMD) in a subject, the device comprising:

an analyzing unit comprising a detection agent for at least one dry or wet AMD diagnostic miRNA molecule, wherein the analyzing unit determines an amount of the at least one dry or wet AMD diagnostic miRNA molecule in a biological sample detected by the detection agent; and

an evaluation unit comprising a processor programmed to:
compare the determined amount obtained from the analyzing unit with a control amount of the at least one dry or wet AMD diagnostic miRNA; and
calculate a likelihood of having or developing DR in a subject, based on results of said comparing the determined amount obtained from the analyzing unit with the control amount.
86. The device according to 85, comprising a display comprising a user interface, the display in electronic communication with the processor of the evaluation unit.
87. A computer system, the system comprising:

a processor; and

memory operably coupled to the processor, wherein the memory programs the processor to:
receive assay data including a determined amount of at least one dry or wet age-related macular degeneration (AMD) diagnostic miRNA in a biological sample from a subject;
compare the amount received with a control amount of the at least one dry or wet AMD diagnostic miRNA; and
calculate a likelihood of having or developing dry or wet AMD in a subject, based on results of said comparing the determined amounts with the control amount.
88. The system according 87, comprising a display comprising a user interface, the display in electronic communication with the processor.
89. A kit for determining a likelihood of having or developing dry or wet age-related macular degeneration (AMD) in a subject, the kit comprising:

a detection agent for at least one dry or wet AMD diagnostic miRNA molecule; and

a device according to 85 or 86.
90. A method of determining a likelihood of having or developing a resistance to anti-VEGF therapy in a subject suffering from age related macular degeneration (AMD), the method comprising:

determining an amount of at least one anti-VEGF therapy resistance diagnostic miRNA in a biological sample from the subject;

comparing the amount of the at least one anti-VEGF therapy resistance diagnostic miRNA with a control amount of the at least one anti-VEGF therapy resistance diagnostic miRNA; and

generating a report indicating a likelihood of having or developing anti-VEGF therapy resistance in the subject based on results of said comparing the amount of the at least one anti-VEGF therapy resistance diagnostic miRNA with the control amount of the at least one anti-VEGF therapy resistance diagnostic miRNA.
91. The method according to 90, wherein the biological sample is serum.
92. The method according to 90 or 91, wherein the at least one anti-VEGF therapy resistance diagnostic miRNA is selected from the group consisting of: miR-1273, miR-302f, miR-30c, miR-3161, miR-3197, miR-483-3p, miR-550-star, miR-573, miR-577, and combinations thereof
93. The method according to any one of 90-92, comprising obtaining the biological sample from the subject.
94. The method according to any one of 90-93, wherein determining the amount of the at least one anti-VEGF therapy resistance AMD diagnostic miRNA comprises performing quantitative real-time PCR.
95. The method according to 94, wherein the quantitative real-time PCR is multiplexed.
96. The method according to any one of 90-95, wherein the report comprises guidance to a clinician as to a treatment recommendation for the subject based on the likelihood of having or developing anti-VEGF therapy resistance in the subject.
97. The method according to 96, wherein the treatment recommendation is administration of an anti-VEGF antibody.
98. The method according to 96, wherein the treatment recommendation is termination of treatment with an anti-VEGF antibody.
99. The method according to any one of 90-98, comprising inputting the amount of the at least one anti-VEGF therapy resistance diagnostic miRNA into a computer comprising a processor programmed to perform the comparing step, wherein said inputting generates a result for a report.
100. The method according to 99, wherein said generating a report is performed by the computer.
101. The method according to 99 or 100, wherein the report is displayed to an output device at a location remote to the computer.
102. A device for determining a likelihood of having or developing a resistance to anti-VEGF therapy in a subject suffering from age related macular degeneration (AMD), the device comprising:

an analyzing unit comprising a detection agent for at least one anti-VEGF therapy resistance diagnostic miRNA molecule, wherein the analyzing unit determines an amount of the at least one anti-VEGF therapy resistance diagnostic miRNA molecule in a biological sample detected by the detection agent; and

an evaluation unit comprising a processor programmed to:
compare the determined amount obtained from the analyzing unit with a control amount of the at least one anti-VEGF therapy resistance diagnostic miRNA; and
calculate a likelihood of having or developing anti-VEGF therapy resistance in a subject, based on results of said comparing the determined amount obtained from the analyzing unit with the control amount.
103. The device according to 102, comprising a display comprising a user interface, the display in electronic communication with the processor of the evaluation unit.
104. A computer system, the system comprising:

a processor; and

memory operably coupled to the processor, wherein the memory programs the processor to:
receive assay data including a determined amount of at least one anti-VEGF therapy resistance diagnostic miRNA in a biological sample from a subject suffering from age-related macular degeneration (AMD);
compare the amount received with a control amount of the at least one anti-VEGF therapy resistance diagnostic miRNA; and
calculate a likelihood of having or developing anti-VEGF therapy resistance in a subject, based on results of said comparing the determined amounts with the control amount.
105. The system according 104, comprising a display comprising a user interface, the display in electronic communication with the processor.
106. A kit for determining a likelihood of having or developing a resistance to anti-VEGF therapy in a subject suffering from age related macular degeneration (AMD), the kit comprising:

a detection agent for at least one anti-VEGF therapy resistance diagnostic miRNA molecule; and

a device according to 102 or 103.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for.

### Example 1: Circulating microRNAs in Ocular Fluids as a Putative Novel Diagnostic Biomarkers for Diabetic Retinopathy

### Materials and Methods

### Experimental Design

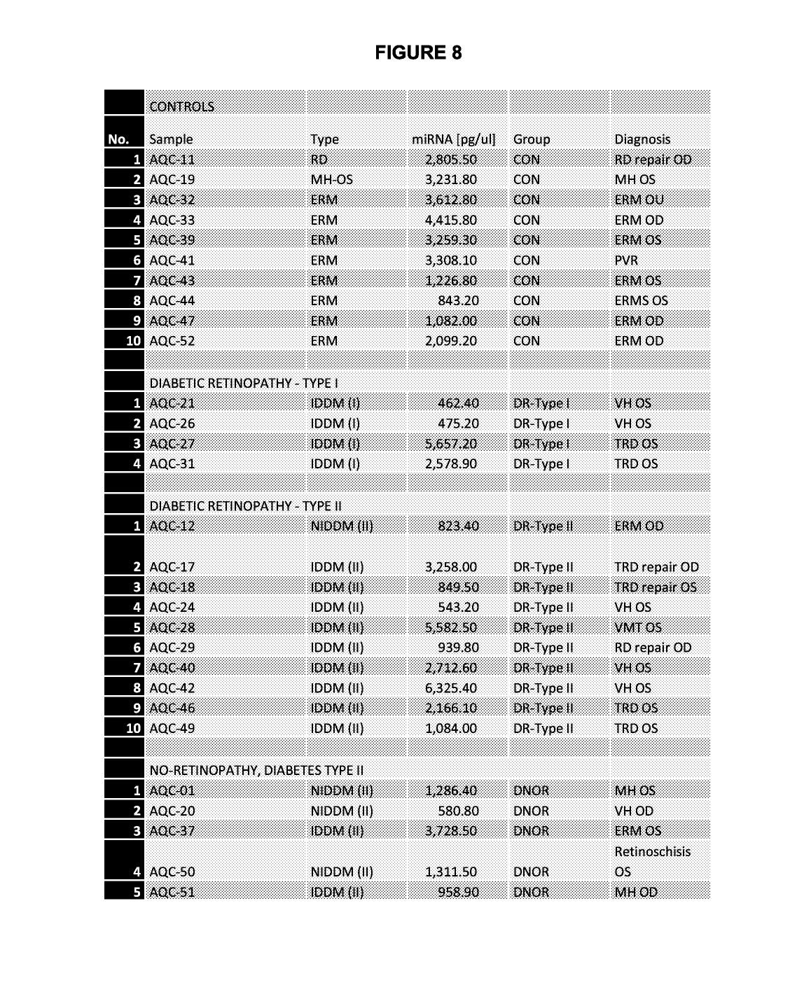

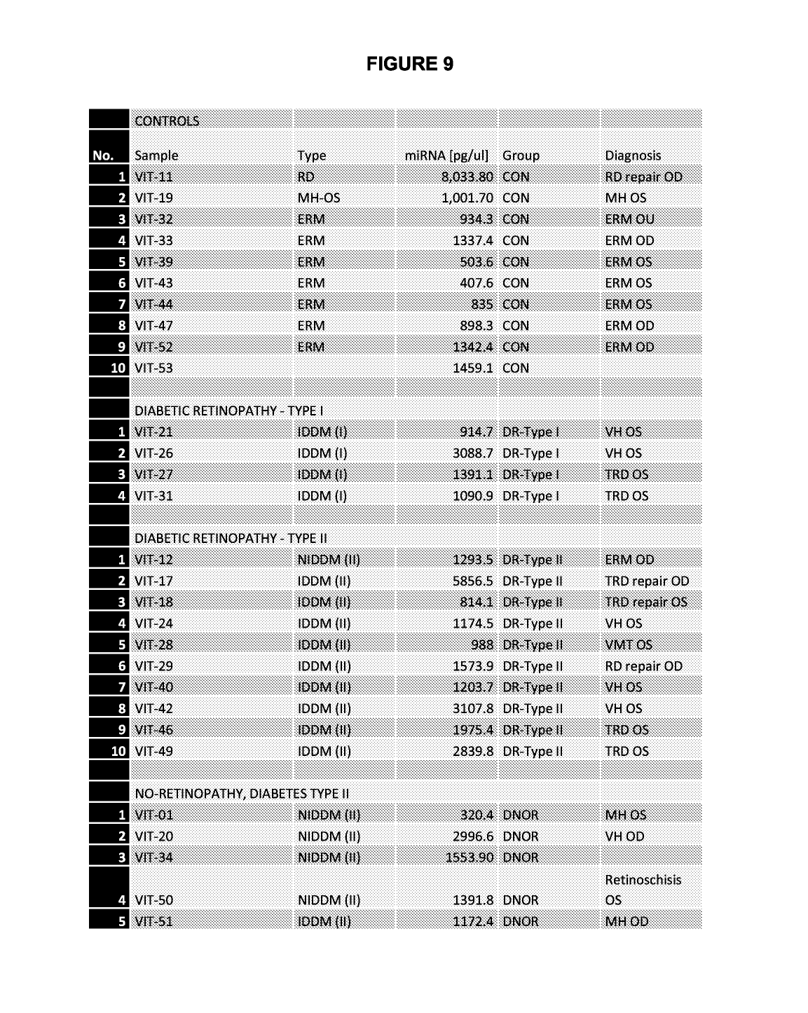

Patients were divided into 4 groups: Group 1: Normals (CON), Group 2: No diabetic retinopathy, Type 2 diabetes mellitus (NDR-II), Group 3: diabetic retinopathy, Type 2 diabetes mellitus (DR-II), Group 4: diabetic retinopathy, Type 1 diabetes mellitus (DR-I). 10 patients were analyzed for CON and DR-II, and 5 patients for NDR-II and 5 for DR-I, which makes 30 patients total (FIGS. 8 and 9). For each patient a sample of aqueous and a sample of vitreous were collected, which represents 60 samples total.

### Human Samples

The samples were from the Clinical “Study of Ocular Fluid, Serum and Urine for Biomarkers of Eye Disease in Patients.” This was an IRB approved, a single-site, investigator-initiated clinical study in the Department of Ophthalmology, UCD. These samples had representation of both genders and various race groups. Age ranged from 30-80 yr old. To assure the anonymity and protection of human subjects the samples were from this point on identified by acquisition number and a record of chronological age, gender, and a description of the case. Inclusion criteria: Patients who were undergoing vitrectomy surgery for retinal disorders (FIGS. 8 and 9). Exclusion criteria: Prior vitreous or retina detachment surgery, prior history of uveitis, endophthalmitis, prior intraocular injections with steroids or anti-VEGF agents, prior cataract surgery less than 6 months ago, history of prior vitreous hemorrhage, penetrating trauma, ruptured globe repair, intraocular tumor, systemic disease including cancer, diabetes, connective tissue disease, current use of systemic steroids or immune modulating agents.

### Sample Collection, Isolation of MicroRNAs, and BioAnalyzer Quantification

Aqueous and vitreous humor samples were collected from DR patients and from control patients during the standard-of-the-care eye surgery. Samples of 100-200u1 were collected, aliquoted, frozen on dry ice, stored at −80° C. and miRNA was isolated using Exiqons' modification of Qiagen's microRNeasy kit. MiRNAs were quantified on BioAnalyzer with Small RNA microfluidics Chip. Blood samples were also collected.

### MicroRNA Affymetrix Microarray Probe Labeling

Probe labeling was done from 10 ng of aqueous and 20 ng of vitreous microRNA samples from the total of 30 aqueous and 30 vitreous samples using FlashTag (Affymetrix) procedure. Labeled probes were hybridized to Affymetrix microRNA Array 3.0 in UC Davis Genome Center Microarray Core Facility using standard procedure (Affymetrix, Santa Clara, Calif.). Total data set included 60 Affymetrix microarrays. Upon scanning of the hybridized and washed Chips, data was obtained in a set of Affymetrix data files (cel, an, pdf).

### Data Normalization

The data sets were analyzed with GeneSpringGX 11 (Agilent, Santa Clara, Calif.) after creating custom Affymetrix miRNA 3.0 technology. Analysis was done separately for each ocular fluid. After importing .cel and .arr files, RMA summarization algorithm was applied with chip-chip normalization using baseline to median of all samples. Probe sets were filtered to their raw signal intensity values, with the criterion that 100% samples in any 1 out of 4 conditions must have values between the 20-100 percentile in the data.

Statistical Analysis: One way ANOVA was used to identify statistically significant genes at the significance level of p≦0.01. The Benjamini-Hochberg post-hoc correction method was too conservative for microarray results with relatively small number of biological replicas for a human study, such as this. No probe sets met this significance level of testing. Therefore, a different set of stringency requirements was applied to reduce the risk of false positives. To identify biologically relevant gene expression changes for each of the time point/treatment conditions, Volcano plot analysis was performed, using a combination of unpaired t-test and asymptotic p-value computation. The Volcano plot is a scatter plot of the fold-change versus the p-value (in −log 10p scale). It is commonly used to simultaneously depict the p-value and the fold-change for gene selection cutoff schemes in microarray data. It may be viewed as a summary of ‘statistical’ significance and ‘biological’ significance over a large number of genes. The standard approach of using a p-value (p≦0.05) as the primary criterion followed by fold change (−1.5≧FC≧1.5) as the secondary criterion was employed to select differentially expressed genes. This approach ensures control of false-positive error and preserves the desired biological significance. Upon first analysis the criterion was relaxed to (−1.2≧FC≧1.2) to be able to capture trends of all the family members of miRNA of interest. Analysis was done for each ocular fluid separately. Each of the groups (DR-Type 2, DR-Type 1 and NDR-Type 2) was compared against control group.

Pathway Analysis with IPA

Pathway and gene network analysis of microRNAs and their target genes was performed using Ingenuity Pathway Analysis (IPA), a web-based software application that enables one to analyze, integrate, and understand the significance of the data, in the context of larger biological systems. IPA is backed by the Ingenuity® Knowledge Database of highly structured, detailed biological findings manually curated by Ph. D. level scientists. MicroRNA Target Filter combines filtering tools and microRNA-mRNA content to provide insight into the biological effects of miRNAs.

### Results

### Bioanalyzer Profiles of Small RNA Populations in Ocular Fluids

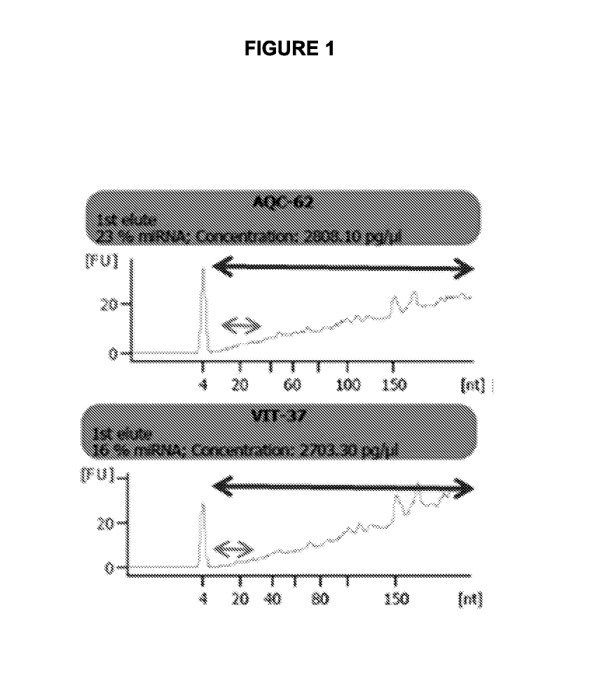

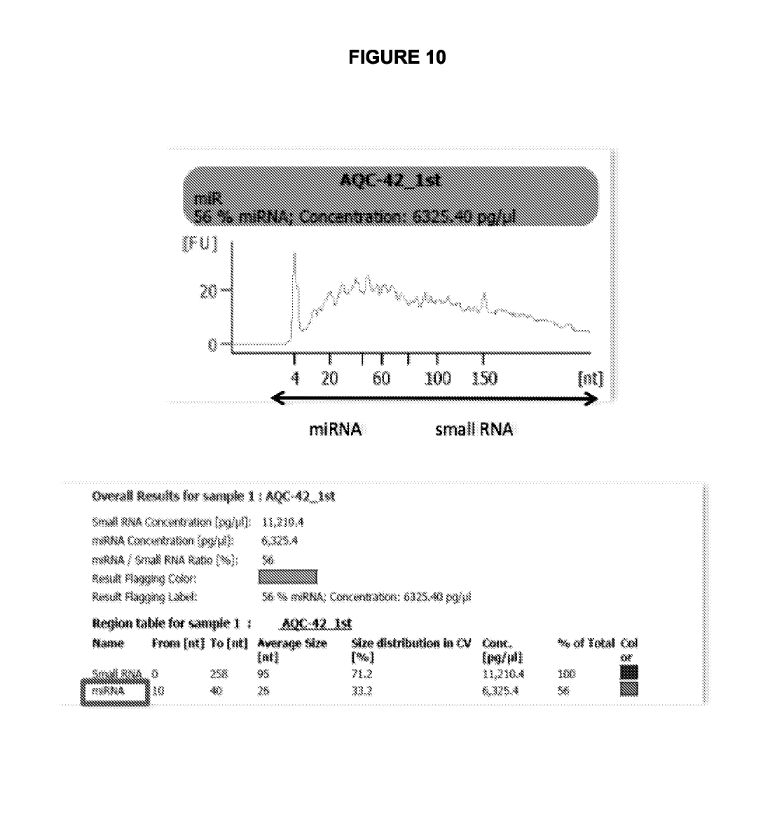

RNA isolation was done from ocular fluids using Exiqon's (modified Qiagens microRNeasy) protocol and QC analysis on BioAnalyzer. RNA was isolated from 100 μl of aqueous and 200 μl vitreous humors. RNA profiles show abundance of small RNA [12,436 pg/μl] representing the population from 0-231 bp (FIG. 1, short arrow; FIG. 10), while microRNAs [2,808.1 pg/μl] are a subset of this profile and represents the population from 10-40 bp (FIG. 1, long arrow; FIG. 10).

Table 1 represents a typical yield of the microRNA and small RNA that was obtained from 100 μl of aqueous, eluted in 30 μl; or from 200 μl of vitreous, eluted in 50 μl of RNase-free water. Quality check and quantification was obtained through analysis on the BioAnalyzer SmallRNA Chips. Generally, the range of microRNA was between 462-5657 [pg/μl] or total yield between 14-170 ng/100 μl for aqueous humor, while the range was from 320-8033 [pg/μl] or total yield of 16-401 ng/200u1 for vitreous humor (FIGS. 8 and 9).

TABLE 1
Typical yield of miRNA and small RNA from aqueous

| Aqueous | From [nt] | To [nt] | Ave size [nt] | Conc. [pg/ul] | % Total |
|---|---|---|---|---|---|
| Small RNA | 0 | 231 | 133 | 12,436.8 | 100 |
| miRNA | 10 | 40 | 28 | 2,808.1 | 23 |

The Most Abundant miRNAs in Aqueous and Vitreous

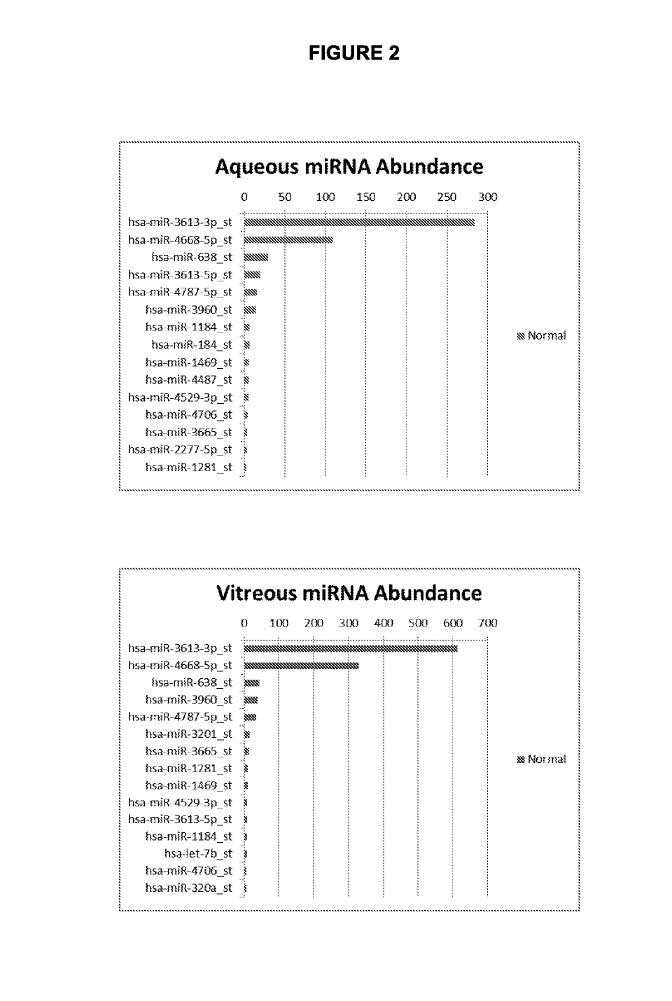

FIG. 2 shows the top 15 most abundant miRNA in aqueous and top 15 most abundant miRNA in vitreous found in control samples. The most abundant microRNA species in these two ocular chambers were very similar. The 3 most abundant microRNAs for both aqueous and vitreous were miR-3613-3p, miR-4668-5p and miR-638. Some of the unique miRNAs at this abundance level were miR-184 for aqueous, and miR-3201, miR-let-7b and miR-320a for vitreous.

Differentially Expressed miRNAs in Aqueous and Vitreous Humors in Diabetics Compared to Controls

Results for each of the 3 groups of diabetic retinopathy vs. control, and for each tissue examined were tabulated (Tables 2-7). Data analysis was done using Volcano plot, with significance p<0.05. Only select differentially expressed miRNA species are shown, but the full lists of the expressed molecules are provided in Tables 8-11. The highest differentially expressed miRNA family in most of the diabetic aqueous and vitreous was let-7 family (Tables 2-5, and 7). Various family members of miR-320 group were also differentially expressed in both aqueous and vitreous (Tables 2, 3 and 5-7). Another potential biomarker upregulated in aqueous of both DR-II and NDR-II patients was miR-26a (Tables 2 and 3). miR-4488 was upregulated in aqueous of both DR-II and NDR-II, and in vitreous of DR-II (Tables 2, 3 and 5), while miR-4662a was downregulated in aqueous and vitreous of only NDR-II (Table 6). miR-638 was strongly downregulated in aqueous of both DR-I and NDR-II (Tables 3 and 4), while miR-29a was mildly upregulated in both aqueous in vitreous of DR-I (Tables 4 and 7). The miR-548 group was also differentially expressed in different stages and types of diabetic retinopathy compared to controls (Tables 4, 5 and 7).

TABLE 2
Aqueous Humor: DR-II vs. CON, Volcano plot,
p < 0.05, FC > 1.2

| No. | Probe Set ID | FC | p |
|---|---|---|---|
| 1 | hsa-miR-let-7b_st | 2.78 | 0.005 |
| 2 | hsa-miR-2861_st | 1.37 | 0.045 |
| 3 | hsa-miR-4488_st | 1.35 | 0.045 |
| 4 | hsa-miR-320c_st | 1.33 | 0.040 |
| 5 | hsa-miR-26a_st | 1.28 | 0.001 |
| 6 | hsa-miR-4697-3p_st | 1.27 | 0.003 |
| 7 | hsa-miR-296-3p_st | 1.27 | 0.001 |
| 8 | hsa-miR-4274_st | 1.24 | 0.005 |
| 9 | hsa-miR-208a_st | 1.23 | 0.000 |
| 10 | hsa-miR-1185_st | 1.22 | 0.004 |
| 11 | hsa-miR-628-5p_st | 1.21 | 0.004 |
| 12 | hp_hsa-mir-4679-2_x_st | 1.21 | 0.004 |
| 13 | hsa-miR-3942-5p_st | 1.20 | 0.031 |
| 14 | hsa-miR-4707-3p_st | −1.21 | 0.015 |
| 15 | hp_hsa-mir-1910_st | −1.21 | 0.001 |

TABLE 3
Aqueous Humor: NDR-II vs. CON, Volcano plot,
p < 0.05, FC > 1.2

| No. | Probe Set ID | FC | p |
|---|---|---|---|
| 1 | hsa-miR-let-7b_st | 3.57 | 0.001 |
| 2 | hsa-miR-3665_st | 2.86 | 0.003 |
| 3 | hsa-miR-4497_st | 2.51 | 0.004 |
| 4 | hsa-miR-let-7c_st | 2.45 | 0.001 |
| 5 | hsa-miR-320b_st | 2.26 | 0.007 |
| 6 | hsa-miR-4466_st | 2.11 | 0.005 |
| 7 | hsa-miR-4488_st | 1.77 | 0.026 |
| 8 | hsa-miR-320a_st | 1.76 | 0.032 |
| 9 | hsa-miR-1915_st | 1.65 | 0.037 |
| 10 | hsa-miR-3196_st | 1.57 | 0.019 |
| 11 | hsa-miR-3656_st | 1.53 | 0.013 |
| 12 | hsa-miR-320c_st | 1.39 | 0.016 |
| 13 | hsa-miR-26a_st | 1.32 | 0.005 |
| 14 | hsa-miR-4662a-3p_st | −1.32 | 0.029 |
| 15 | hsa-miR-638_st | −2.06 | 0.003 |

TABLE 4
Aqueous Humor: DR-I vs. CON, Volcano plot, p < 0.05, FC > 1.2

| No. | Probe Set ID | FC | p |
|---|---|---|---|
| 1 | hsa-miR-184_st | 3.97 | 0.035 |
| 2 | hsa-miR-let-7b_st | 3.34 | 0.001 |
| 3 | hsa-miR-3202_st | 1.35 | 0.003 |
| 4 | hsa-miR-29a_st | 1.32 | 0.001 |
| 5 | hsa-miR-3942-5p_st | 1.31 | 0.019 |
| 6 | hsa-miR-1267_st | 1.30 | 0.002 |
| 7 | hsa-miR-4665-5p_st | 1.27 | 0.010 |
| 8 | hsa-miR-548c-3p_st | 1.26 | 0.005 |
| 9 | hsa-miR-let-7d-star_st | 1.26 | 0.038 |
| 10 | hsa-miR-26b-star_st | 1.26 | 0.008 |
| 11 | hsa-miR-1973_st | 1.25 | 0.022 |
| 12 | hp_hsa-mir-519d_x_st | −1.26 | 0.011 |
| 13 | hp_hsa-mir-3153_x_st | −1.26 | 0.027 |
| 14 | hp_hsa-mir-4530_st | −1.27 | 0.046 |
| 15 | hsa-miR-638_st | −2.39 | 0.019 |

TABLE 5
Vitreous humor: DR-II vs. CON, Volcano plot, p < 0.05, FC > 1.2

| No. | Probe Set ID | FC (abs) | p |
|---|---|---|---|
| 1 | hsa-miR-let-7b_st | 6.26 | 0.006 |
| 2 | hsa-miR-320a_st | 3.57 | 0.017 |
| 3 | hsa-miR-320b_st | 3.26 | 0.027 |
| 4 | hsa-miR-320c_st | 2.83 | 0.018 |
| 5 | hsa-miR-let-7c_st | 2.36 | 0.031 |
| 6 | hsa-miR-200c_st | 2.22 | 0.043 |
| 7 | hsa-miR-762_st | 2.14 | 0.002 |
| 8 | hsa-miR-4488_st | 1.99 | 0.002 |
| 9 | hp_hsa-mir-124-3_s_st | 1.77 | 0.015 |
| 10 | hsa-miR-4516_st | 1.76 | 0.038 |
| 11 | hsa-miR-4695-5p_st | 1.58 | 0.000 |
| 12 | hsa-miR-let-7a_st | 1.20 | 0.015 |
| 13 | hsa-miR-766_st | −1.37 | 0.001 |
| 14 | hsa-miR-4445-star_st | −1.39 | 0.027 |
| 15 | hp_hsa-mir-548ai_st | −1.85 | 0.042 |

TABLE 6
Vitreous humor: NDR-II vs CON, Volcano plot,
p < 0.05, FC > 1.2

| No. | Probe Set ID | FC (abs) | p |
|---|---|---|---|
| 1 | hsa-miR-3613-5p_st | 6.18 | 0.016 |
| 2 | hsa-miR-423-5p_st | 1.50 | 0.003 |
| 3 | hp_hsa-mir-4679-2_x_st | 1.38 | 0.016 |
| 4 | hp_hsa-mir-1254-2_x_st | 1.36 | 0.002 |
| 5 | hp_hsa-mir-559_st | 1.35 | 0.006 |
| 6 | hsa-miR-3162-3p_st | 1.35 | 0.029 |
| 7 | hsa-miR-760_st | 1.33 | 0.004 |
| 8 | hp_hsa-mir-9-1_st | 1.25 | 0.005 |
| 9 | hsa-miR-320e_st | 1.25 | 0.027 |
| 10 | hsa-miR-3668_st | 1.25 | 0.001 |
| 11 | hsa-miR-4707-3p_st | −1.26 | 0.016 |
| 12 | hsa-miR-4662a-3p_st | −1.27 | 0.010 |
| 13 | hsa-miR-4696_st | −1.28 | 0.022 |
| 14 | hp_hsa-mir-941-3_s_st | −1.33 | 0.002 |
| 15 | hsa-miR-3201_st | −2.55 | 0.041 |

TABLE 7
Vitreous humor: DR-I vs CON, Volcano plot, p < 0.05, FC > 1.2

| No. | Probe Set ID | FC | p |
|---|---|---|---|
| 1 | hsa-miR-let-7b_st | 17.09 | 0.004 |
| 2 | hsa-miR-92a_st | 6.47 | 0.004 |
| 3 | hsa-miR-320c_st | 5.16 | 0.003 |
| 4 | hsa-miR-185_st | 4.99 | 0.001 |
| 5 | hsa-miR-320a_st | 4.97 | 0.008 |
| 6 | hsa-miR-320b_st | 4.88 | 0.015 |
| 7 | hsa-miR-16_st | 4.71 | 0.020 |
| 8 | hsa-miR-let-7c_st | 4.44 | 0.004 |
| 9 | hsa-miR-4488_st | 2.66 | 0.002 |
| 10 | hsa-miR-let-7d_st | 2.23 | 0.012 |
| 11 | hsa-miR-423-5p_st | 2.14 | 0.001 |
| 12 | hsa-miR-20a_st | 2.12 | 0.024 |
| 13 | hsa-miR-29a_st | 1.23 | 0.011 |
| 14 | hsa-miR-let-7b-star_st | 1.23 | 0.028 |
| 15 | hp_hsa-mir-548f-1_x_st | −1.29 | 0.004 |
| 16 | hsa-miR-3910_st | −1.42 | 0.017 |
| 17 | hsa-miR-3128_st | −1.49 | 0.014 |
| 18 | hp_hsa-mir-520h_s_st | −1.68 | 0.008 |
| 19 | hp_hsa-mir-548ai_x_st | −2.92 | 0.016 |
| 20 | hsa-miR-4668-5p_st | −3.49 | 0.039 |
| 21 | hp_hsa-mir-548ai_st | −3.82 | 0.010 |
| 22 | hsa-miR-3201_st | −4.82 | 0.001 |

TABLE 8
NDR-II vs. CON, Volcano plot, p < 0.05,
FC > 1.2 (complete results)

| Probe Set ID | Fold Change | p < 0.05 |
|---|---|---|
| hsa-miR-let-7b_st | 3.57 | 0.003 |
| hsa-miR-3665_st | 2.86 | 0.004 |
| hsa-miR-4497_st | 2.51 | 0.001 |
| hsa-miR-let-7c_st | 2.45 | 0.007 |
| hsa-miR-320b_st | 2.26 | 0.005 |
| hsa-miR-4466_st | 2.11 | 0.026 |
| hsa-miR-4488_st | 1.77 | 0.032 |
| hsa-miR-320a_st | 1.76 | 0.037 |
| hsa-miR-1915_st | 1.65 | 0.019 |
| hsa-miR-3196_st | 1.57 | 0.013 |
| hsa-miR-3656_st | 1.53 | 0.029 |
| hsa-miR-2861_st | 1.46 | 0.035 |
| hsa-miR-3178_st | 1.43 | 0.002 |
| hsa-miR-320c_st | 1.39 | 0.016 |
| hsa-miR-149-star_st | 1.38 | 0.020 |
| hsa-miR-4739_st | 1.37 | 0.001 |
| hsa-miR-214_st | 1.35 | 0.002 |
| hsa-miR-330-5p_st | 1.33 | 0.004 |
| hsa-miR-26a_st | 1.32 | 0.005 |
| hsa-miR-4754_st | 1.29 | 0.010 |
| hsa-miR-940_st | 1.29 | 0.001 |
| hsa-miR-4795-5p_st | 1.28 | 0.007 |
| hsa-miR-1200_st | 1.28 | 0.019 |
| hsa-miR-4727-5p_st | 1.26 | 0.006 |
| hsa-miR-576-3p_st | 1.25 | 0.024 |
| hsa-miR-4296_st | 1.25 | 0.006 |
| hsa-miR-4432_st | 1.25 | 0.009 |
| hsa-miR-920_st | 1.24 | 0.008 |
| hsa-miR-520g_st | 1.24 | 0.008 |
| hsa-miR-3189-3p_st | 1.23 | 0.041 |
| hsa-miR-let-7f-1-star_st | 1.23 | 0.021 |
| hsa-miR-3668_st | 1.23 | 0.010 |
| hsa-miR-122-star_st | 1.23 | 0.020 |
| hsa-miR-374c_st | 1.22 | 0.016 |
| hsa-miR-298_st | 1.22 | 0.005 |
| hsa-miR-4682_st | 1.22 | 0.012 |
| hsa-miR-4670-3p_st | 1.22 | 0.008 |
| hsa-miR-4693-3p_st | 1.22 | 0.025 |
| hsa-miR-502-3p_st | 1.22 | 0.015 |
| hsa-miR-2355-3p_st | 1.21 | 0.026 |
| hsa-miR-3918_st | 1.20 | 0.006 |
| hsa-miR-2277-3p_st | 1.20 | 0.024 |
| hsa-miR-574-5p_st | −1.20 | 0.027 |
| hsa-miR-323-3p_st | −1.21 | 0.020 |
| hsa-miR-1227_st | −1.21 | 0.037 |
| hsa-miR-548al_st | −1.21 | 0.030 |
| hsa-miR-4650-3p_st | −1.21 | 0.039 |
| hsa-miR-607_st | −1.21 | 0.035 |
| hsa-miR-3913-3p_st | −1.22 | 0.011 |
| hsa-miR-3591-5p_st | −1.22 | 0.017 |
| hsa-miR-3180-5p_st | −1.22 | 0.006 |
| hsa-miR-548b-3p_st | −1.22 | 0.019 |
| hsa-miR-3937_st | −1.23 | 0.031 |
| hsa-miR-3156-3p_st | −1.23 | 0.029 |
| hsa-miR-483-3p_st | −1.24 | 0.034 |
| hsa-miR-4469_st | −1.26 | 0.008 |
| hsa-miR-4258_st | −1.28 | 0.016 |
| hsa-miR-4719_st | −1.29 | 0.002 |
| hsa-miR-4662a-3p_st | −1.32 | 0.003 |
| hsa-miR-638_st | −2.06 | 0.024 |

TABLE 9
Vitreous humor: DR-I vs CON, Volcano plot, p <
0.05, FC > 1.2 (complete results)

| Probe Set ID | Fold Change | p < 0.05 |
|---|---|---|
| hsa-miR-let-7b_st | 17.09 | 0.004 |
| hsa-miR-92a_st | 6.47 | 0.004 |
| hsa-miR-320c_st | 5.16 | 0.003 |
| hsa-miR-185_st | 4.99 | 0.001 |
| hsa-miR-320a_st | 4.97 | 0.008 |
| hsa-miR-320b_st | 4.88 | 0.015 |
| hsa-miR-16_st | 4.71 | 0.020 |
| hsa-miR-let-7c_st | 4.44 | 0.004 |
| hsa-miR-4488_st | 2.66 | 0.002 |
| hsa-miR-let-7d_st | 2.23 | 0.012 |
| hsa-miR-423-5p_st | 2.14 | 0.001 |
| hsa-miR-20a_st | 2.12 | 0.024 |
| hsa-miR-221_st | 1.82 | 0.002 |
| hsa-miR-425_st | 1.78 | 0.029 |
| hsa-miR-20b_st | 1.74 | 0.022 |
| hsa-miR-4429_st | 1.62 | 0.024 |
| hsa-miR-4440_st | 1.55 | 0.029 |
| hsa-miR-4793-3p_st | 1.55 | 0.041 |
| hsa-miR-19b_st | 1.53 | 0.028 |
| hsa-miR-4695-5p_st | 1.50 | 0.002 |
| hsa-miR-574-3p_st | 1.47 | 0.046 |
| hsa-miR-15a_st | 1.46 | 0.026 |
| hsa-miR-22_st | 1.42 | 0.027 |
| hsa-miR-502-3p_st | 1.42 | 0.025 |
| hsa-miR-126_st | 1.41 | 0.032 |
| hsa-miR-4484_st | 1.40 | 0.044 |
| hsa-miR-4499_st | 1.37 | 0.004 |
| hsa-miR-339-3p_st | 1.36 | 0.041 |
| hsa-miR-1268b_st | 1.35 | 0.009 |
| hsa-miR-4803_st | 1.33 | 0.003 |
| hsa-miR-4421_st | 1.33 | 0.001 |
| hsa-miR-449b-star_st | 1.32 | 0.002 |
| hsa-miR-34c-5p_st | 1.32 | 0.004 |
| hsa-miR-1471_st | 1.31 | 0.003 |
| hsa-miR-514_st | 1.31 | 0.005 |
| hsa-miR-4521_st | 1.30 | 0.008 |
| hsa-miR-302d_st | 1.30 | 0.003 |
| hsa-miR-1224-5p_st | 1.30 | 0.007 |
| hsa-miR-4685-5p_st | 1.29 | 0.014 |
| hsa-miR-151-3p_st | 1.27 | 0.044 |
| hsa-miR-381_st | 1.26 | 0.008 |
| hsa-miR-411-star_st | 1.26 | 0.006 |
| hsa-miR-4431_st | 1.25 | 0.016 |
| hsa-miR-195-star_st | 1.25 | 0.005 |
| hsa-miR-3688-3p_st | 1.25 | 0.048 |
| hsa-miR-33a_st | 1.25 | 0.021 |
| hsa-miR-3158-5p_st | 1.24 | 0.007 |
| hsa-miR-620_st | 1.23 | 0.006 |
| hsa-miR-29a_st | 1.23 | 0.011 |
| hsa-miR-3197_st | 1.23 | 0.013 |
| hsa-miR-2277-3p_st | 1.23 | 0.018 |
| hsa-miR-let-7b-star_st | 1.23 | 0.028 |
| hsa-miR-3189-5p_st | 1.23 | 0.017 |
| hsa-miR-4795-3p_st | 1.21 | 0.008 |
| hsa-miR-3166_st | 1.21 | 0.016 |
| hsa-miR-219-5p_st | 1.20 | 0.010 |
| hsa-miR-3179_st | 1.20 | 0.006 |
| hsa-miR-545_st | 1.20 | 0.004 |
| hsa-miR-3679-3p_st | −1.22 | 0.007 |
| hsa-miR-103b_st | −1.24 | 0.038 |
| hsa-miR-4269_st | −1.24 | 0.031 |
| hsa-miR-4699-3p_st | −1.25 | 0.049 |
| hsa-miR-4485_st | −1.25 | 0.010 |
| hsa-miR-1181_st | −1.25 | 0.030 |
| hsa-miR-298_st | −1.29 | 0.006 |
| hsa-miR-1234_st | −1.30 | 0.014 |
| hsa-miR-1255b_st | −1.34 | 0.045 |
| hsa-miR-3910_st | −1.42 | 0.017 |
| hsa-miR-3128_st | −1.49 | 0.014 |
| hsa-miR-4668-5p_st | −3.49 | 0.039 |
| hsa-miR-3201_st | −4.82 | 0.001 |

TABLE 10
Vitreous humor: DR-II vs. CON, Volcano plot, p <
0.05, FC > 1.2 (complete results)

| Probe Set ID | Fold Change | p < 0.05 |
|---|---|---|
| hsa-miR-let-7a_st | 1.20 | 0.015 |
| hsa-miR-let-7b_st | 6.26 | 0.006 |
| hsa-miR-let-7c_st | 2.36 | 0.031 |
| hsa-miR-1275_st | 1.27 | 0.005 |
| hsa-miR-200c_st | 2.22 | 0.043 |
| hsa-miR-320a_st | 3.57 | 0.017 |
| hsa-miR-320b_st | 3.26 | 0.027 |
| hsa-miR-320c_st | 2.83 | 0.018 |
| hsa-miR-3689b-star_st | 1.21 | 0.005 |
| hsa-miR-3940-5p_st | 1.45 | 0.007 |
| hsa-miR-423-5p_st | 1.20 | 0.022 |
| hsa-miR-4463_st | 1.42 | 0.026 |
| hsa-miR-4488_st | 1.99 | 0.002 |
| hsa-miR-4516_st | 1.76 | 0.038 |
| hsa-miR-4683_st | 1.20 | 0.012 |
| hsa-miR-4695-5p_st | 1.58 | 0.000 |
| hsa-miR-4801_st | 1.23 | 0.010 |
| hsa-miR-574-3p_st | 1.46 | 0.025 |
| hsa-miR-762_st | 2.14 | 0.002 |
| hsa-miR-1273g_st | −1.21 | 0.018 |
| hsa-miR-4284_st | −1.25 | 0.002 |
| hsa-miR-4434_st | −1.20 | 0.024 |
| hsa-miR-4445-star_st | −1.39 | 0.027 |
| hsa-miR-4756-3p_st | −1.21 | 0.008 |
| hsa-miR-766_st | −1.37 | 0.001 |

TABLE 11
Vitreous humor: NDR-II vs CON, Volcano plot,
p < 0.05, FC > 1.2

| Probe Set ID | Fold Change | p < 0.05 |
|---|---|---|
| hsa-miR-3613-5p_st | 6.18 | 0.016 |
| hsa-miR-423-5p_st | 1.50 | 0.003 |
| hsa-miR-3162-3p_st | 1.35 | 0.029 |
| hsa-miR-760_st | 1.33 | 0.004 |
| hsa-miR-4440_st | 1.31 | 0.019 |
| hsa-miR-4758-5p_st | 1.26 | 0.021 |
| hsa-miR-3194-3p_st | 1.25 | 0.004 |
| hsa-miR-320e_st | 1.25 | 0.027 |
| hsa-miR-3668_st | 1.25 | 0.001 |
| hsa-miR-126_st | 1.22 | 0.025 |
| hsa-miR-4668-3p_st | 1.21 | 0.033 |
| hsa-miR-4494_st | 1.21 | 0.041 |
| hsa-miR-4433_st | 1.21 | 0.049 |
| hsa-miR-3689b-star_st | 1.20 | 0.017 |
| hsa-miR-3121-3p_st | −1.20 | 0.028 |
| hsa-miR-4727-3p_st | −1.21 | 0.050 |
| hsa-miR-4756-3p_st | −1.22 | 0.038 |
| hsa-miR-548g_st | −1.22 | 0.023 |
| hsa-miR-1233_st | −1.22 | 0.019 |
| hsa-miR-487b_st | −1.22 | 0.041 |
| hsa-miR-4505_st | −1.22 | 0.028 |
| hsa-miR-4261_st | −1.24 | 0.041 |
| hsa-miR-3679-3p_st | −1.25 | 0.002 |
| hsa-miR-4661-5p_st | −1.25 | 0.029 |
| hsa-miR-4707-3p_st | −1.26 | 0.016 |
| hsa-miR-4662a-3p_st | −1.27 | 0.010 |
| hsa-miR-4696_st | −1.28 | 0.022 |
| hsa-miR-3201_st | −2.55 | 0.041 |

Candidate miRNA Biomarkers Upregulated in Both Aqueous and Vitreous

Let-7 Expression in Ocular Fluids.

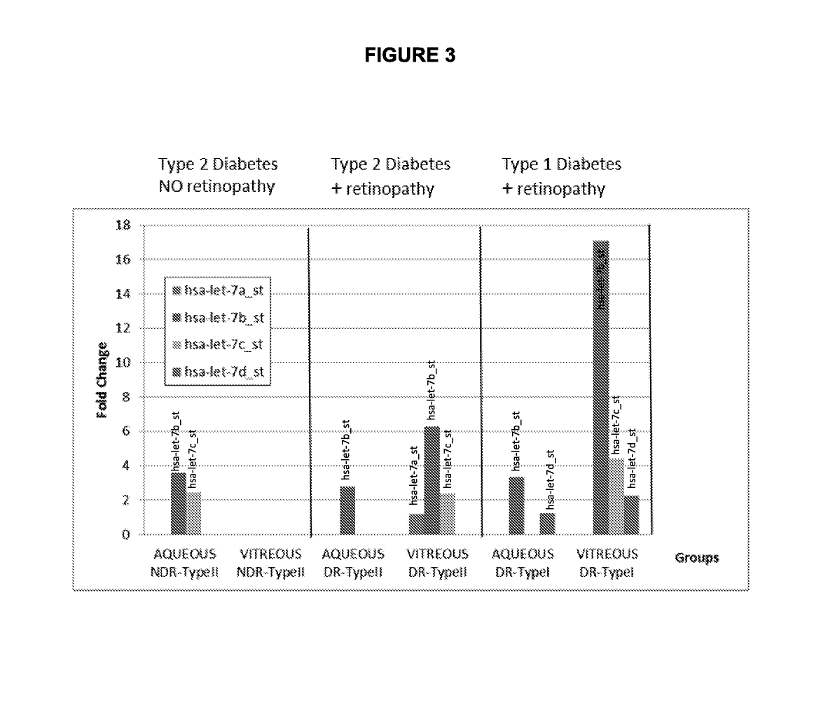

Fold Change of miR-let-7a, miR-let-7b, miR-let-7c and miR-let-7d observed in a particular diabetic group compared to the control group is shown in FIG. 3. Overexpression of miR-let-7b and miR-let-7c in aqueous was observed in Type 2 diabetes patients with no retinopathy (FIG. 3, NDR-Type II). Overexpression of miR-let-7b in aqueous and miR-let-7a, miR-let-7b and miR-let-7c in vitreous was observed in Type 2 diabetes patients with retinopathy (FIG. 3, DR-Type II). Overexpression of miR-let-7b and miR-let-7d in aqueous and miR-let-7b, miR-let-7c and miR-let-7d in vitreous was observed in Type 1 diabetes patients with retinopathy (FIG. 3, DR-Type I).

miR-320 Expression in Ocular Fluids.

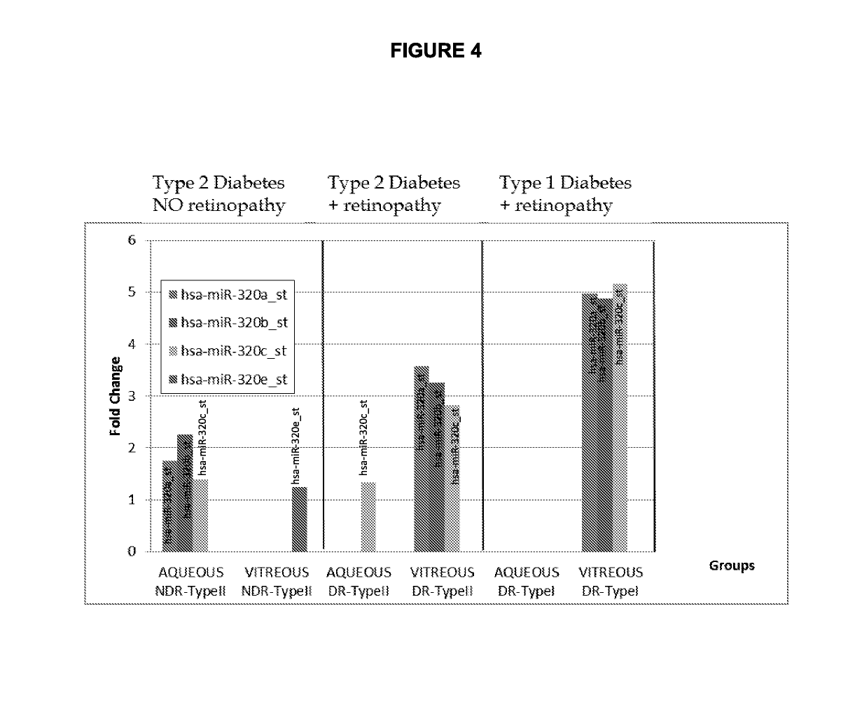

MiR-320 family members were present in both chambers in two groups with Type II diabetes, with or without retinopathy, but only in vitreous of Type I diabetic patients (FIG. 4). In the aqueous of NDR samples there was overexpression of miR-320a, b and c, while in the vitreous of NDR-Type II sample there was overexpression of miR-320e. In the DR-Type II samples, overexpression of miR-320c was observed in aqueous, while miR-320a, b and c were prevalent in vitreous. In DR-Type I samples, only vitreous showed overexpression of miR-320a, b and c, while aqueous did not show overexpression of any of this family members.

### IPA Pathway Analysis

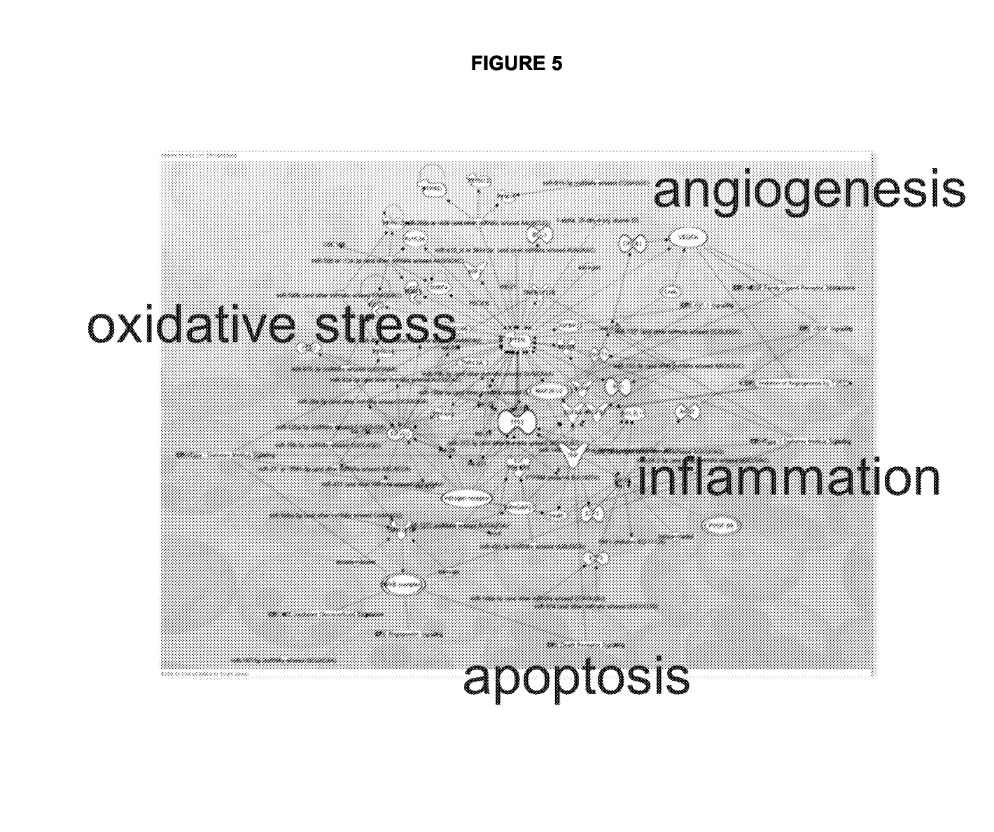

Ingenuity Pathways Analysis was done using 30 highest level expressing miRNAs from aqueous and vitreous, most of them being common for both chambers (FIG. 5). Circulating miRNA that are ubiquitously present in ocular chambers target primarily genes regulated by p53 and TGF-beta. These genes are major regulators of oxidative stress, angiogenesis, inflammation and apoptosis.

### Pathway Analysis of Let-7

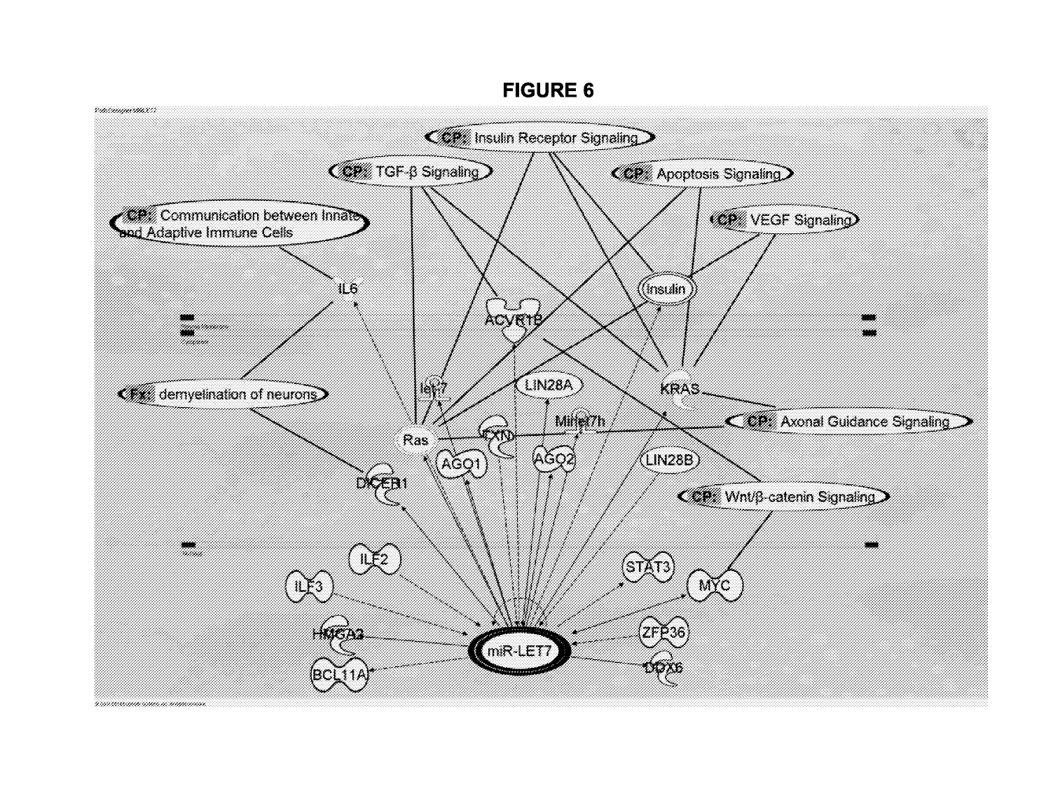

FIG. 6 illustrates how dysregulation of even one miRNA family can have widespread consequences for many different cellular functions. Current estimates indicate that more than one third of the cellular transcriptome is regulated by miRNAs. Represented in FIG. 6 is a partial interactome of the let-7 family of miRNAs and some of the target genes and biological pathways that they regulate: TGF-β, Insulin Receptor, Apoptosis and VEGF Receptor Signaling.

Pathway Analysis of miR-320

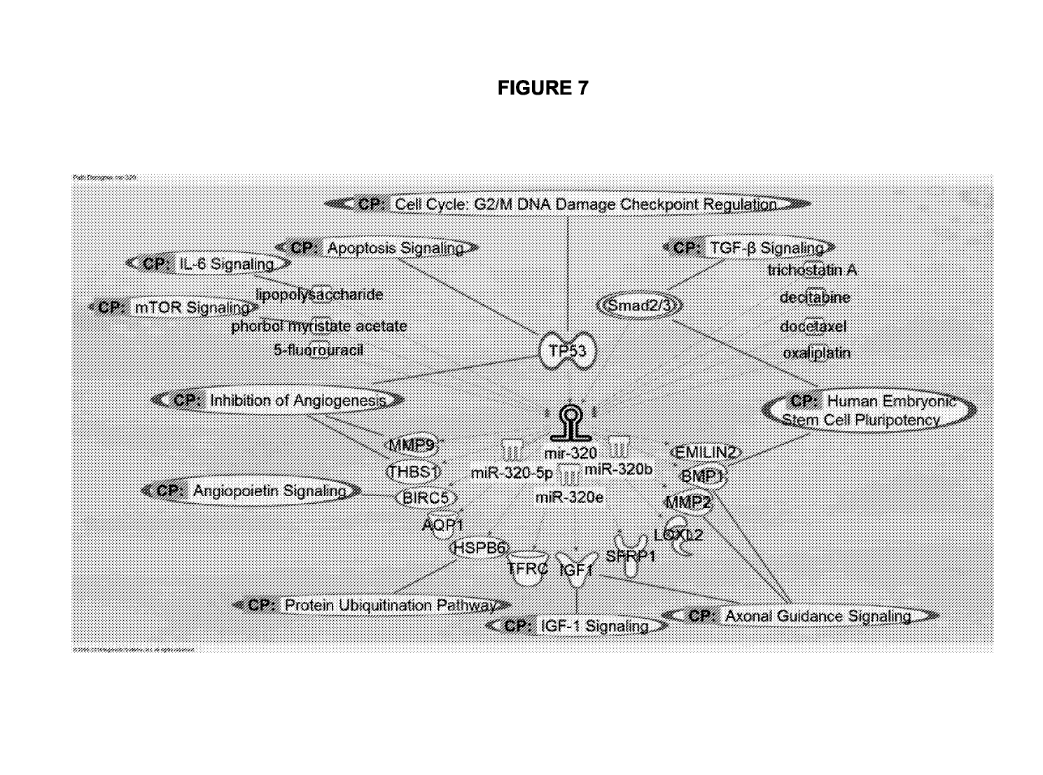

MiR-320 family has been assigned biological process by Gene Ontology (GO) classification as part of cellular response to glucose stimulus. According to IPA summary, it regulates: SFRP1, THBS1, EMILIN2, LOXL2, BMP1, MMP2, MMP9, BIRCS, IGF1, HSPB6, AQP4, AQP1, TGF beta, and it is regulated by: p53 and Smad2/3, which is a downstream from TGF-beta (FIG. 7). Several small molecules can modulate miR-320 expression: phorbol myristate acetate, docetaxel, oxaliplatin, 5-fluorouracil, decitabine, lipopolysaccharide and trichostatin A. MiR-320 binds to BIRCS, TFRC, and has the role in cell in apoptosis, migration, cell death, proliferation, and signaling in several diseases including non-insulin-dependent diabetes mellitus.

The results showed that the most abundant microRNA in both aqueous and vitreous was miR-3613-3p. IPA analysis revealed that top pathways that genes regulated by this microRNA are involved in are, p53 signaling and Human Embryonic Stem Cell Pluripotency. The results show that multiple microRNAs in both aqueous and vitreous are differentially expressed in DR, and according to IPA analysis, the main pathways targeted by miRNAs in ocular fluids are the TGF-beta pathway, Wnt/beta-catenin Signaling, p53 signaling and Human Embryonic Stem Cell Pluripotency.

### Example 2: Circulatory microRNA in Serum as Potential Biomarkers for AMD

### Materials and Methods

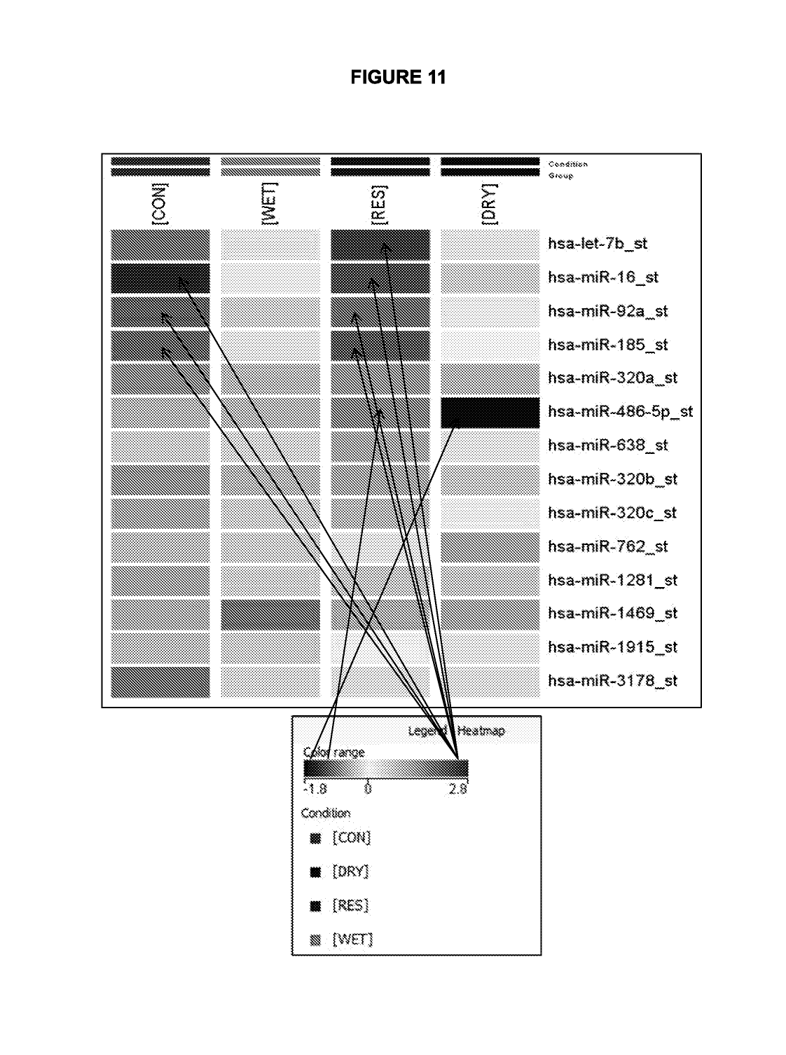

Serum samples of 40 patients from 4 different groups were collected. Group 1 were 10 age-matched normals with no ocular or systemic disease, Group 2 were 10 patients with category 3 high risk dry AMD, Group 3 were 10 new unilateral exudative AMD patients treated with Lucentis or Avastin, and Group 4 were 10 patients resistant to anti-VEGF therapy. RNA was isolated using modified Qiagen microRNeasy procedure, and quantitated on BioAnalyzer's small RNA chips. Microarray miRNA analysis of serum samples was performed on 4 samples from each group and ran on Affymetrix 2.0 miRNA arrays. The data sets were analyzed with GeneSpring software (FIG. 11). Statistical analysis was done by ANOVA. Confirmatory quantitative real-time PCR (qPCR) was used on the select set of biomarker candidates for the full sample set of each group. RNA spike-ins and melting curve analysis served as quality control, as well as serial dilutions to detect possible bias. Three microRNAs with stable expression in serum served as reference genes for data normalization. Student's t-test was used for statistical analysis.

### Results

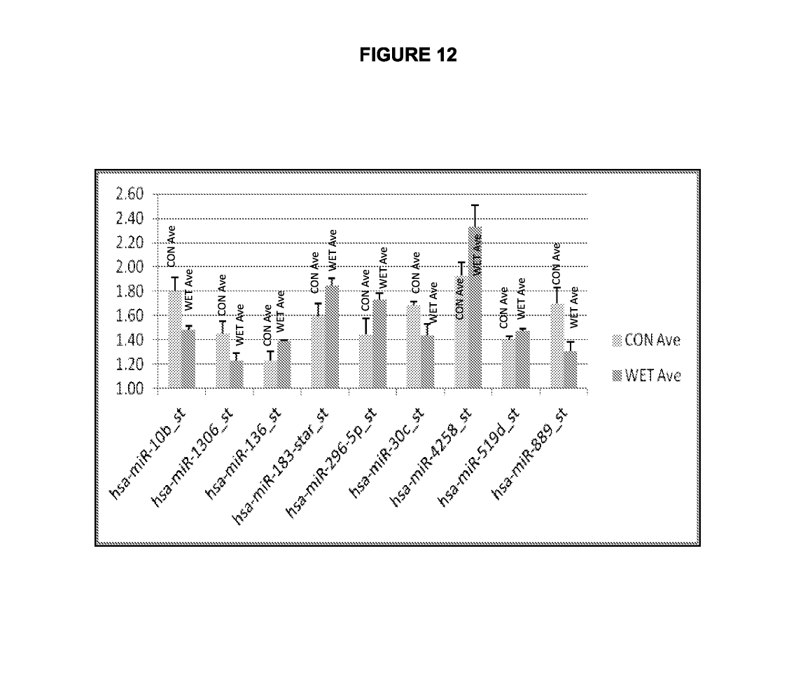

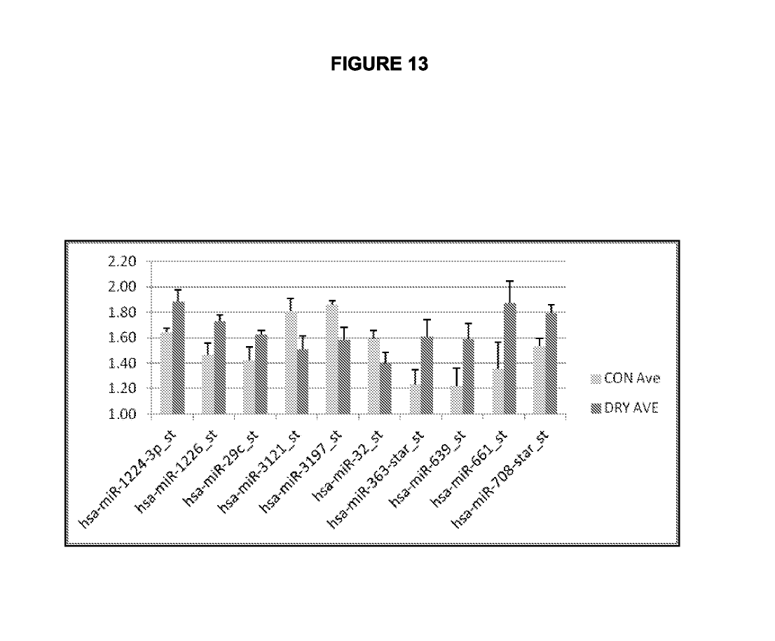

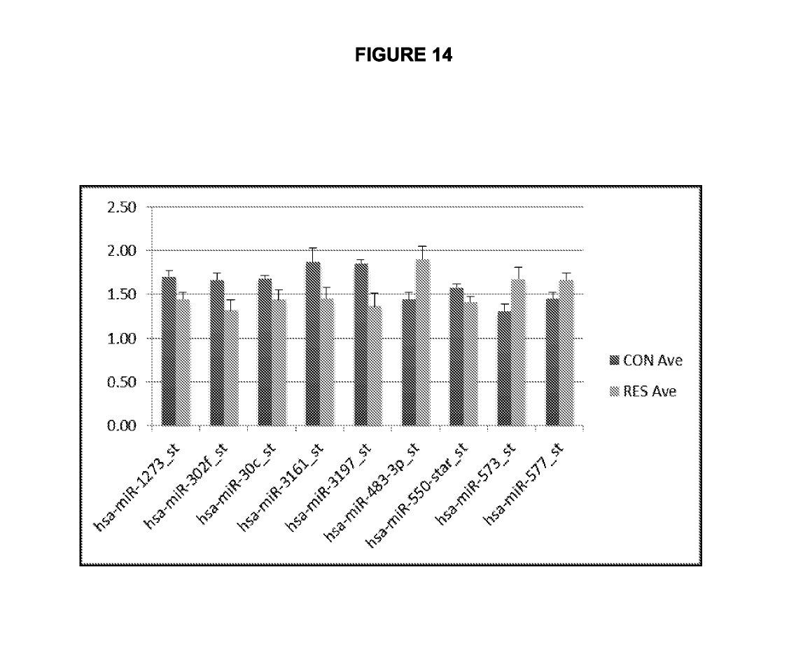

Several statistically significant differences in miRNA expression were seen when comparing the 3 AMD groups with age-matched control group (FIG. 11). Both increase and decrease in expression levels were observed. The miRNAs that exhibited statistically significant differences in expression levels are shown in FIG. 12-14. For wet AMD vs. control, out of a total of 9 dysregulated miRNAs, the miRNA showing the largest up-regulation was miR-4258, and the miRNA showing the largest down-regulation was miR-889 (FIG. 12). For Dry AMD vs. control, out of a total of 10 dysregulated miRNAs, the miRNA showing the largest up-regulation was miR-661, and the miRNA showing the largest down-regulation was miR-3121 (FIG. 13). For resistant AMD vs. control, out of a total of 9 dysregulated miRNAs, the miRNA showing the largest up-regulation was miR-483, and the miRNA showing the largest down-regulation was miR-3161 (FIG. 14). Subsequent qPCR was used to verify findings from microarray analysis.

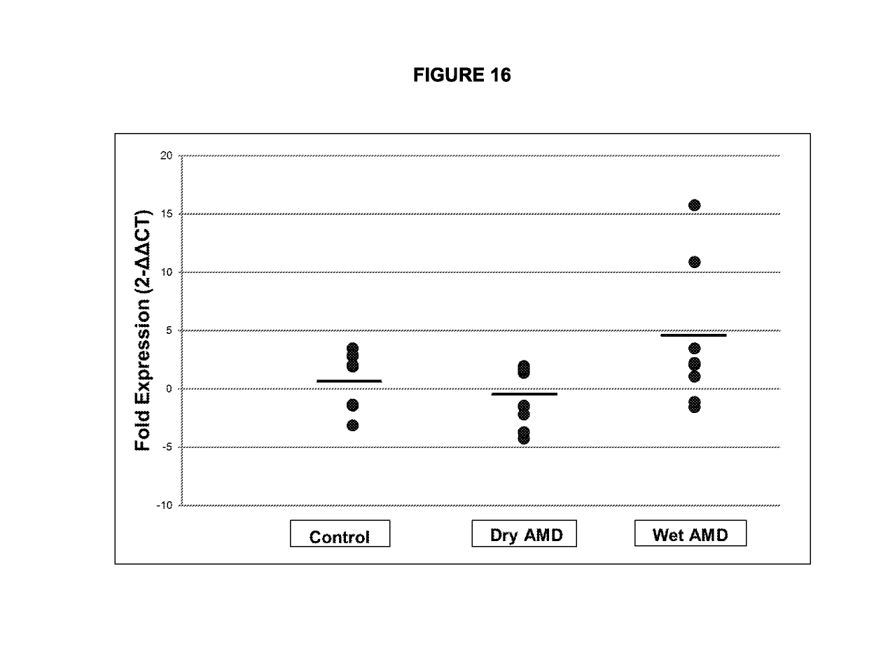

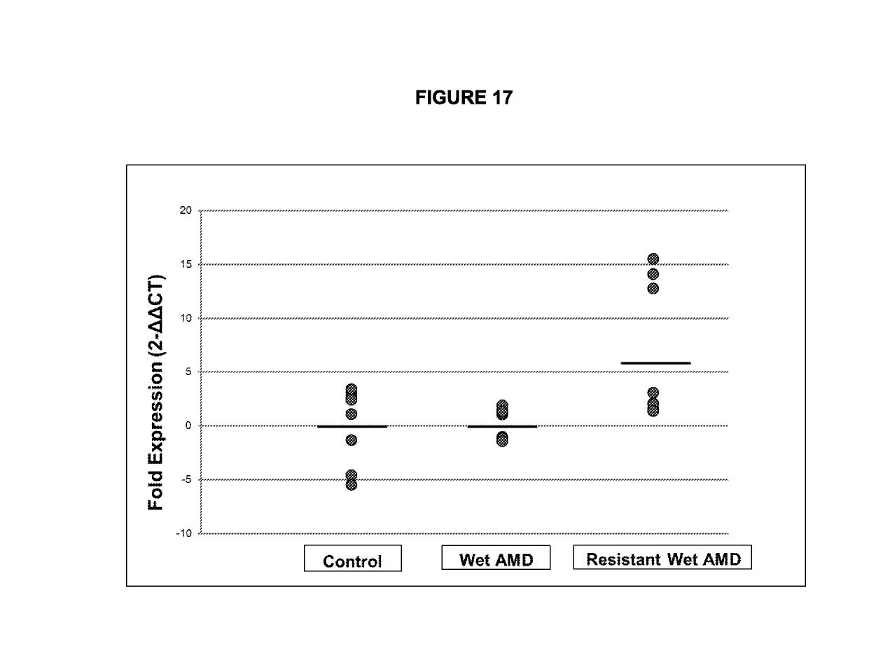

Two noteworthy miRNAs are MiR-let-7b and miR-486. MiR-let-7b was highly up-regulated in patients resistant to anti-VEGF treatment while miR-486 was down-regulated in dry AMD compared to the other groups. Quantitative Real-Time PCR was subsequently performed to verify these findings (FIGS. 16 and 17). miR-486 expression was confirmed to be down-regulated in dry AMD, while it was shown to be up-regulated in wet AMD (FIG. 16). It was also confirmed that some patients who were resistant to anti-VEGF therapy showed increased MiR-let-7b production in serum (p<0.05). MiR-let-7b expression was 6.8 fold higher in serum from patients with AMD resistant to anti-VEGF therapy than patients with wet AMD (p<0.01).

This study indicated several potential biomarkers for dry and wet AMD, as well as potential biomarkers of resistance to anti-VEGF treatment. The miRNA levels that were significantly altered between the groups tested may offer diagnostic and therapeutic benefits.

### Example 3: Comparative Expression Analysis in Plasma of Circulating microRNAs Identified in Ocular Fluid as Putative Biomarkers for Diabetic Retinopathy

The following experiments were conducted to establish whether miRNA biomarkers, which were identified in the ocular fluids as putative biomarkers for diabetic retinopathy, are also differentially expressed in plasma.

### Materials and Methods

Plasma, aqueous and vitreous samples were collected from 10 controls (macular pucker or macular hole patients) and 20 patients with diabetes undergoing vitreoretinal surgery [diabetic retinopathy, Type 1 diabetes mellitus (DR-I) (also referred to in the Figures as Diabetes Mellitus, Type I (DMI)); Diabetes Mellitus, Type II, with proliferative retinopathy (DMII-PDR) (a sub-group of diabetic retinopathy, Type 2 diabetes mellitus (DR-II)); Diabetes Mellitus, Type II, with non-proliferative retinopathy (DMII-NPDR) (a sub-group of diabetic retinopathy, Type 2 diabetes mellitus (DR-II)); and Diabetes Mellitus, Type II, with no retinopathy (DMII-noDR)]. Samples were stored at −80° C. MicroRNAs from plasma were isolated using Exiqon RNA purification kit, quantified on BioAnalyzer, labeled with FlashTag kit and profiled on Affymetrix GeneChip miRNA 3.0 microarrays. Data analysis was performed using Expression Console (EC) and Transcription Analysis Console (TAC) (Affymetrix) and for pathway analysis Ingenuity Pathway Analysis software (IPA) was used. qPCR using Taqman assays was used as an independent technique to confirm the candidate microRNAs.

Additional analysis was performed via qPCR using Taqman assays.

### Results

Comparison of circulatory microRNAs from aqueous and vitreous humor with corresponding plasma samples from the same patients showed that statistically significant dysregulation of some of the members from the let-7 and miR-320 families can be detected (p<0.05; −1.5>Fold Change (FC)>1.5).

In the DMI (DR-I) group select let-7 microRNAs are upregulated in the ocular fluids (FC=1.5−50) and downregulated in plasma (FC=−1.41 to −4.38), while miR-320 microRNAs are upregulated both in vitreous (FC=5.1) and plasma (FC=1.24).

The former DR-II group was further refined into DMII-PDR and DMII-NPDR.

In DMII-PDR, select let-7 family members are upregulated in ocular fluids, and downregulated in plasma. Members of the miR-320 family are also upregulated in ocular fluids, while not significantly dysregulated in plasma.

In DMII-NPDR, select let-7 family members are upregulated in ocular fluids, while not significantly dysregulated in plasma. MiR-320 family shows upregulation in aqueous, and downregulation in plasma.

In DMII-noDR (also referred to as DNOR) patients exhibited upregulation of let-7 family members mainly in aqueous, less in vitreous, and some in plasma. Mir-320 family members were mainly upregulated in aqueous, some in vitreous, and in plasma exhibited a downregulation trend.

Pathway analysis of the let-7 family of miRNAs showed that some of the biological pathways that they regulate are TGF-beta, Insulin Receptor Signaling, Apoptosis and VEGF Receptor Signaling. MiR-320 family has been assigned biological process by GO as cellular response to glucose stimulus and has a role in apoptosis, migration, proliferation, and signaling in diabetes mellitus.

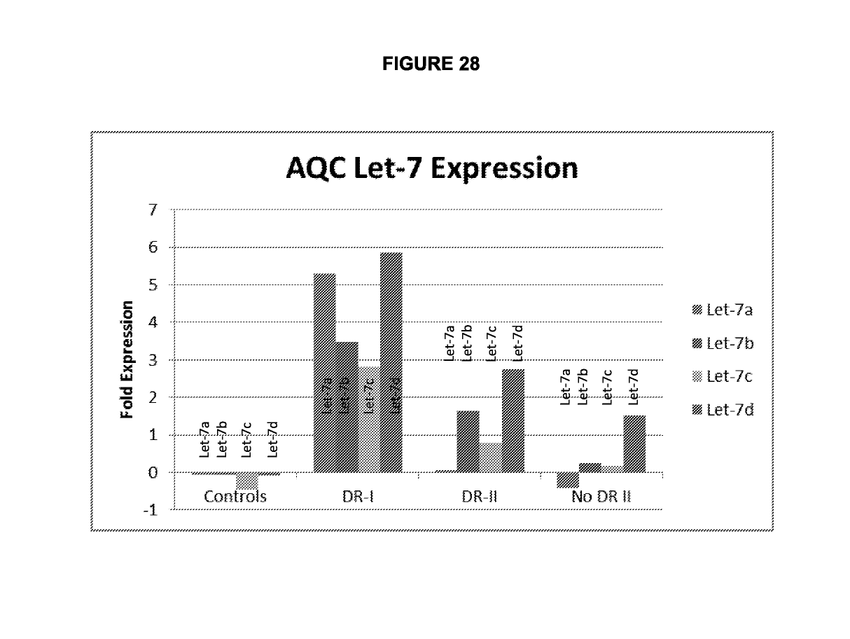

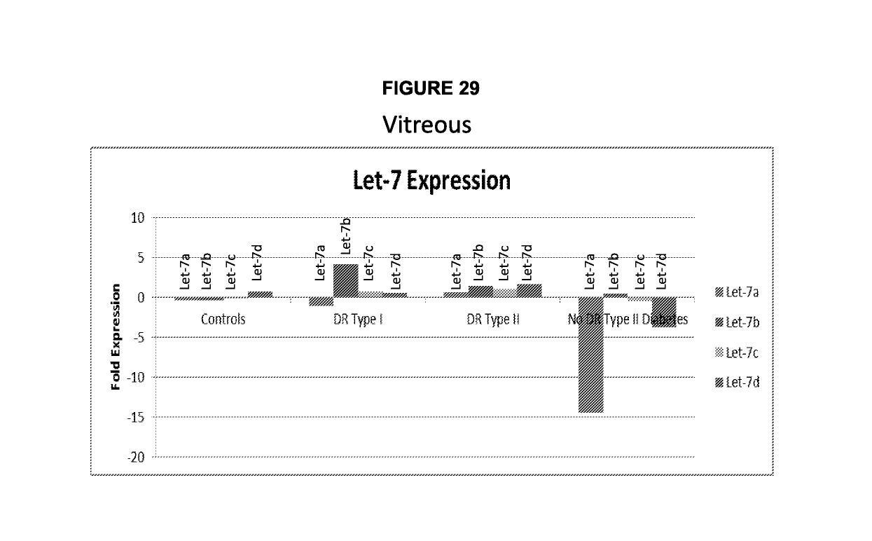

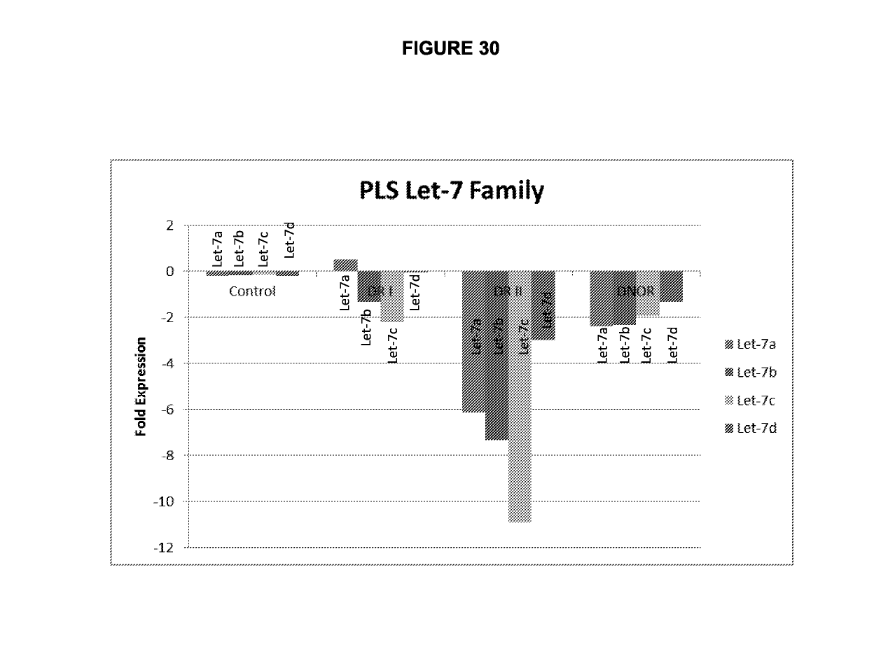

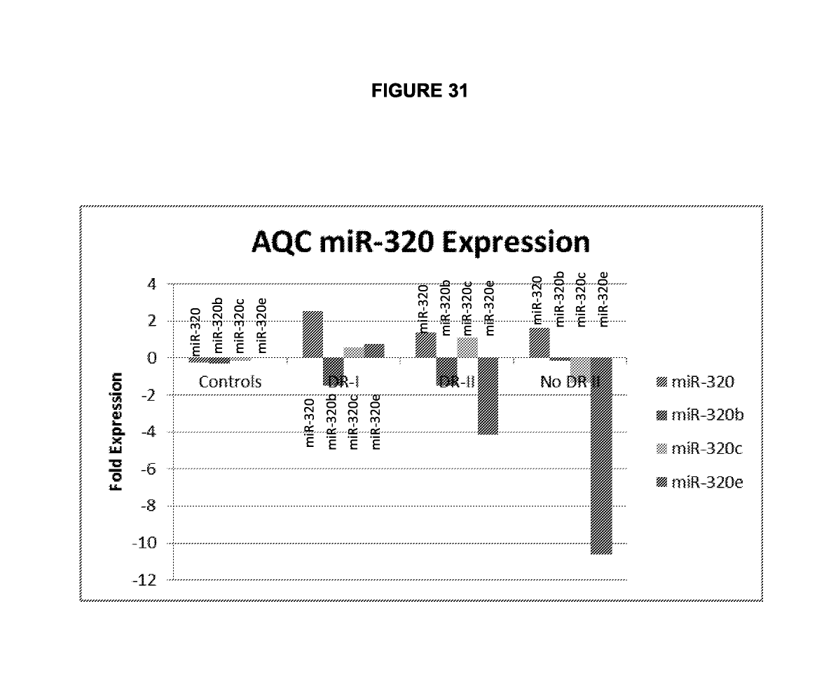

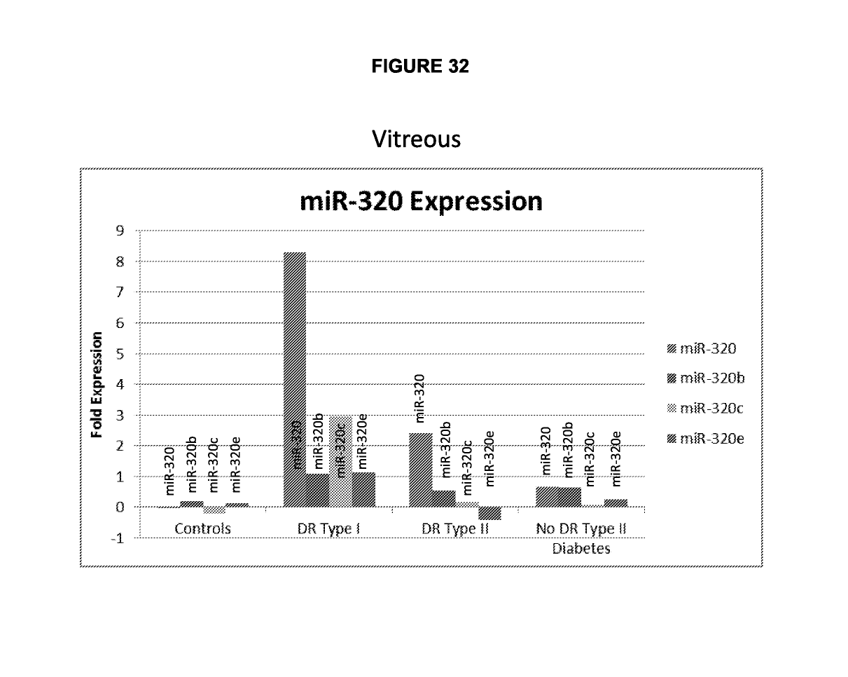

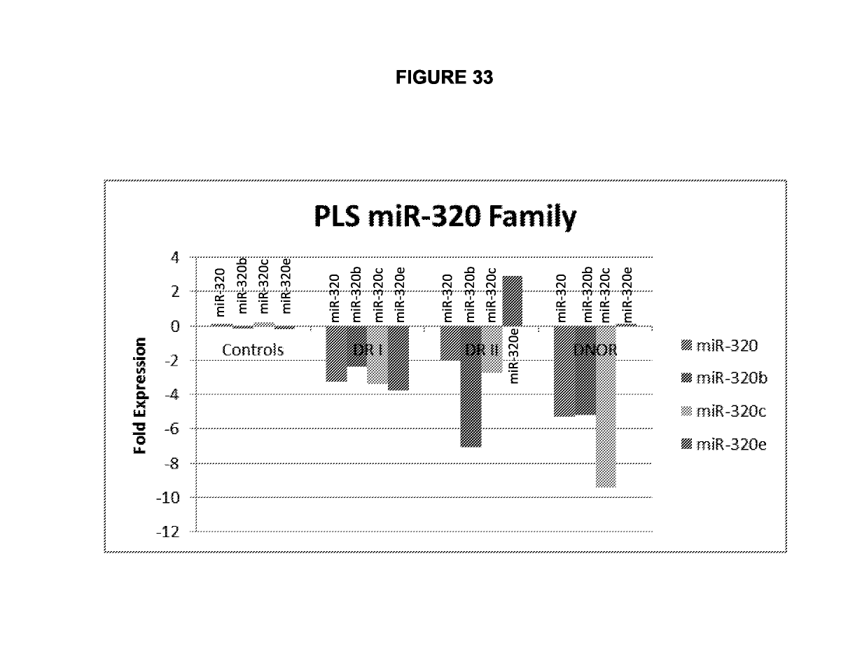

Expression levels as determined via qPCR for select let-7 and miR-320 family miRNAs are provided in FIGS. 28-33.

### Example 4: Circulating microRNAs in Plasma as Putative Biomarkers of Diabetic Retinopathy

The goal of this study was to identify plasma microRNAs (miRNA) as a prognostic tools in diabetic patients for development of diabetic retinopathy.

### Materials and Methods

Plasma samples from 28 patients undergoing vitrectomy were collected at the time of the procedure.
Group 1: Type I diabetics with retinopathy (n=4)
Group 2: Type II diabetics with retinopathy (n=10): proliferative (7) and non-proliferative (3),
Group 3: Type II diabetics without retinopathy (n=4),
Group 4: Control patients without known diabetes (n=10)

MiRNA's were isolated using the Exiqon RNA purification kit for fluid samples. Presence of miRNA was confirmed and quantified using a BioAnalyzer and small RNA chip. Samples were labeled with FlashTag procedure and hybridized to Affymetrix GeneChip 3.0. Normalization of the .cel files using RMA algorithm was performed using Affymetrix software Expression Console. ANOVA was used to determine statistical significance of differentially expressed miRNAs, using Transcriptome Analysis Console (TAC) software. Significant changes (p<0.05), and fold change (FC) increases (FC>1.5) and decreases (FC<−1.5) of miRNA expression were identified between sample groups and controls.

### Results

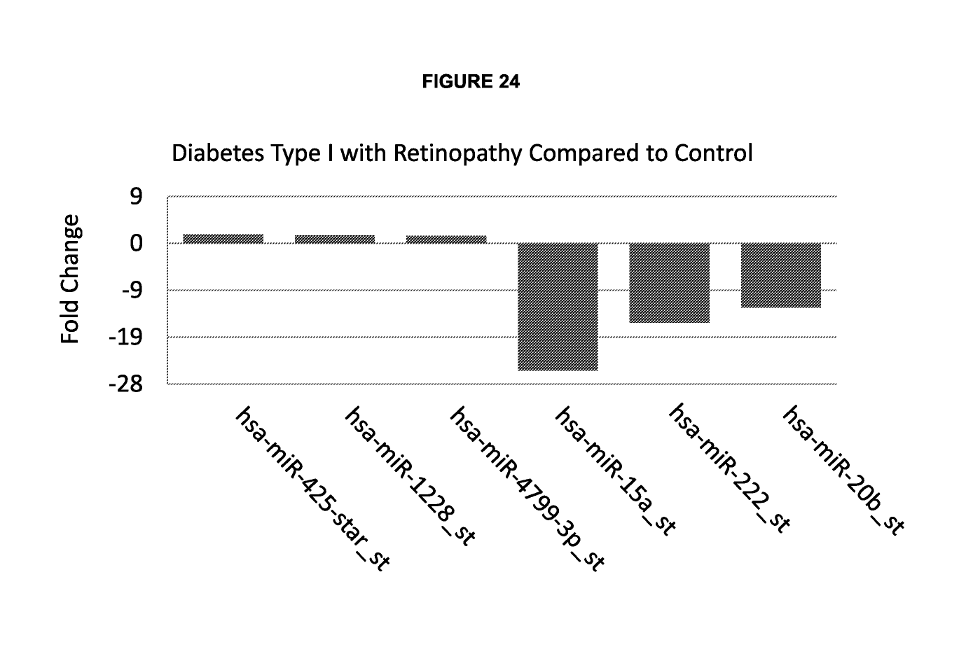

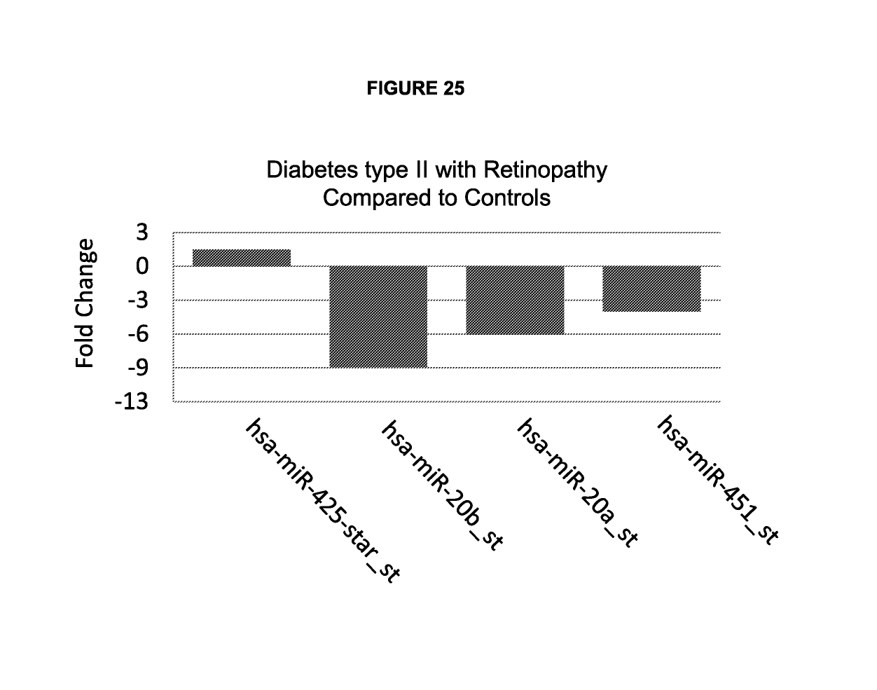

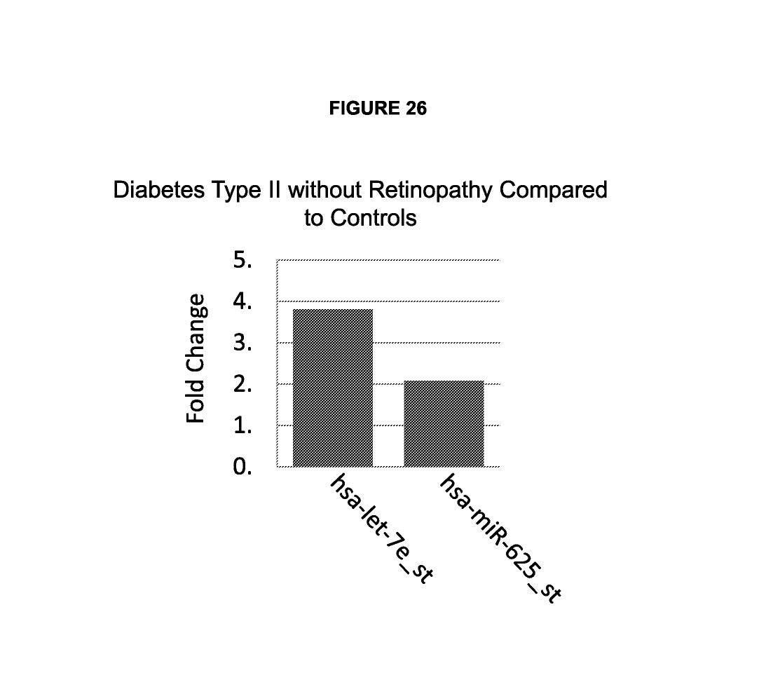

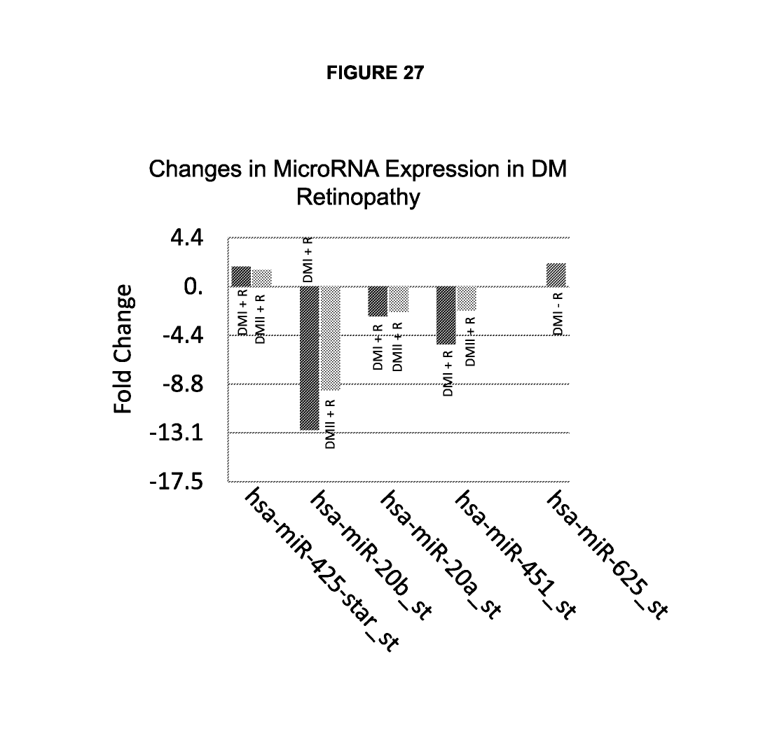

Results are provided in FIGS. 24-27. As shown, there are unique differences in the miRNA profiles of the plasma in patients with diabetic retinopathy (Type I and II), diabetics without retinopathy, and non-diabetics. Accordingly, such miRNAs may serve as prognostic plasma biomarkers of diabetic retinopathy. For example, patients with Type I diabetes with retinopathy exhibited an increase in expression of hsa-miR-425-star, hsa-miR-1228, and hsa-miR-4799-3p in plasma compared to controls; and a decrease in expression of hsa-miR-15a, hsa-miR-222, and hsa-miR-20b in plasma compared to controls (FIGS. 24 and 27). Patients with type II diabetes with retinopathy exhibited an increase in expression of hsa-miR-425-star and a decrease in expression of hsa-miR-20b, hsa-miR-20a, and hsa-miR-451 in plasma compared to controls (FIGS. 25 and 27). Patients with type II diabetes without retinopathy exhibited an increase in expression of hsa-let-7e and hsa-miR-625 in plasma compared to controls (FIGS. 26 and 27).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of determining a likelihood of having or developing diabetic retinopathy (DR) in a subject, the method comprising:
determining an amount of at least one DR diagnostic miRNA in a biological sample from the subject;
comparing the amount of the at least one DR diagnostic miRNA with a control amount of the at least one DR diagnostic miRNA; and
generating a report indicating a likelihood of having or developing DR in the subject based on results of said comparing the amount of the at least one DR diagnostic miRNA with the control amount of the at least one DR diagnostic miRNA.

2. The method according to claim 1, wherein the biological sample is aqueous humor, vitreous humor, or plasma.

3. The method according to claim 2, wherein the biological sample is aqueous humor and the at least one DR diagnostic miRNA is selected from the group consisting of: miR-let-7b, miR-let-7d, miR-320c, miR-26a, miR-4488, miR-638, miR-29a, and combinations thereof.

4. The method according to claim 2, wherein the biological sample is vitreous humor and the at least one DR diagnostic miRNA is selected from the group consisting of: miR-let-7a, miR-let-7b, miR-let-7c, miR-let-7d, miR-320a, miR-320b, miR-320c, miR-4488, miR-29a, and combinations thereof.

5.-12. (canceled)

13. The method according to claim 1, comprising obtaining the biological sample from the subject.

14. The method according to claim 1, wherein determining the amount of the at least one DR diagnostic miRNA comprises performing quantitative real-time PCR.

15. The method according to claim 14, wherein the quantitative real-time PCR is multiplexed.

16. The method according to claim 1, wherein the report comprises guidance to a clinician as to a treatment recommendation for the subject based on the likelihood of having or developing DR in the subject.

17. The method according to claim 16, wherein the treatment recommendation is control of blood glucose, blood pressure and/or blood cholesterol.

18. The method according to claim 16, wherein the treatment recommendation is administration of an anti-VEGF antibody.

19. The method according to claim 16, wherein the treatment recommendation is scatter laser treatment.

20. The method according to claim 16, wherein the treatment recommendation is focal laser treatment.

21. The method according to claim 1, comprising inputting the amount of the at least one DR diagnostic miRNA into a computer comprising a processor programmed to perform the comparing step, wherein said inputting generates a result for a report.

22.-23. (canceled)

24. A method of determining efficacy of a therapy for treatment of diabetic retinopathy (DR) in a subject, the method comprising:
determining an amount of at least one DR diagnostic miRNA in a biological sample from the subject prior to administration of the therapy;
administering the therapy;
determining an amount of the least one DR diagnostic miRNA in a biological sample from the subject following administration of the therapy;
comparing the amount of the at least one DR diagnostic miRNA in a biological sample prior to administration of the therapy with the amount of the at least one DR diagnostic miRNA in a biological sample following administration of the therapy; and
generating a report indicating efficacy of the therapy based on results of said comparing.

25.-52. (canceled)

53. A method of determining a likelihood of having or developing dry or wet age-related macular degeneration (AMD) in a subject, the method comprising:
determining an amount of at least one dry or wet AMD diagnostic miRNA in a biological sample from the subject;
comparing the amount of the at least one dry or wet AMD diagnostic miRNA with a control amount of the at least one dry or wet AMD diagnostic miRNA; and
generating a report indicating a likelihood of having or developing dry or wet AMD in the subject based on results of said comparing the amount of the at least one dry or wet AMD diagnostic miRNA with the control amount of the at least one dry or wet AMD diagnostic miRNA.

54. The method according to claim 53, wherein the biological sample is serum.

55. (canceled)

56. The method according to claim 53, wherein the at least one dry or wet AMD diagnostic miRNA is at least one wet AMD diagnostic miRNA, and wherein the at least one wet AMD diagnostic miRNA is selected from the group consisting of: miR-10b, miR-1306, miR-136, miR-183-star, miR-296-5p, miR-30c, miR-4258, miR-519d, miR-889, and combinations thereof.

57. (canceled)

58. The method according to claim 53, wherein the at least one dry or wet AMD diagnostic miRNA is at least one dry AMD diagnostic miRNA, and wherein the at least one dry AMD diagnostic miRNA is selected from the group consisting of: miR-1224-3p, miR-1226, miR-29c, miR-3121, miR-3197, miR-32, miR-363-star, miR-639, miR-661, miR-708-star, and combinations thereof.

59. (canceled)

60. The method according to any one of claim 53, wherein determining the amount of the at least one dry or wet AMD diagnostic miRNA comprises performing quantitative real-time PCR.

61. The method according to claim 60, wherein the quantitative real-time PCR is multiplexed.

62.-89. (canceled)

90. A method of determining a likelihood of having or developing a resistance to anti-VEGF therapy in a subject suffering from age related macular degeneration (AMD), the method comprising:
determining an amount of at least one anti-VEGF therapy resistance diagnostic miRNA in a biological sample from the subject;
comparing the amount of the at least one anti-VEGF therapy resistance diagnostic miRNA with a control amount of the at least one anti-VEGF therapy resistance diagnostic miRNA; and
generating a report indicating a likelihood of having or developing anti-VEGF therapy resistance in the subject based on results of said comparing the amount of the at least one anti-VEGF therapy resistance diagnostic miRNA with the control amount of the at least one anti-VEGF therapy resistance diagnostic miRNA.

91.-106. (canceled)


SEQUENCE LISTING (124 sequences)

SEQ ID NO: 1 - RNA, 80 nt, Homo sapiens
ugggaugagguaguagguuguauaguuuuagggucacacccaccacugggagauaacuauacaaucuacugucuuuccua

SEQ ID NO: 2 - RNA, 72 nt, Homo sapiens
agguugagguaguagguuguauaguuuagaauuacaucaagggagauaacuguacagccuccuagcuuuccu

SEQ ID NO: 3 - RNA, 74 nt, Homo sapiens
gggugagguaguagguuguauaguuuggggcucugcccugcuaugggauaacuauacaaucuacugucuuuccu

SEQ ID NO: 4 - RNA, 22 nt, Homo sapiens
ugagguaguagguuguauaguu

SEQ ID NO: 5 - RNA, 21 nt, Homo sapiens
cuauacaaucuacugucuuuc

SEQ ID NO: 6 - RNA, 22 nt, Homo sapiens
cuguacagccuccuagcuuucc

SEQ ID NO: 7 - RNA, 83 nt, Homo sapiens
cggggugagguaguagguugugugguuucagggcagugauguugccccucggaagauaacuauacaaccuacugccuucccug

SEQ ID NO: 8 - RNA, 22 nt, Homo sapiens
ugagguaguagguugugugguu

SEQ ID NO: 9 - RNA, 22 nt, Homo sapiens
cuauacaaccuacugccuuccc

SEQ ID NO: 10 - RNA, 84 nt, Homo sapiens
gcauccggguugagguaguagguuguaugguuuagaguuacacccugggaguuaacuguacaaccuucuagcuuuccuuggagc

SEQ ID NO: 11 - RNA, 22 nt, Homo sapiens
ugagguaguagguuguaugguu

SEQ ID NO: 12 - RNA, 22 nt, Homo sapiens
cuguacaaccuucuagcuuucc

SEQ ID NO: 13 - RNA, 87 nt, Homo sapiens
ccuaggaagagguaguagguugcauaguuuuagggcagggauuuugcccacaaggagguaacuauacgaccugcugccuuucuuagg

SEQ ID NO: 14 - RNA, 22 nt, Homo sapiens
agagguaguagguugcauaguu

SEQ ID NO: 15 - RNA, 22 nt, Homo sapiens
cuauacgaccugcugccuuucu

SEQ ID NO: 16 - RNA, 82 nt, Homo sapiens
gcuucgcuccccuccgccuucucuucccgguucuucccggagucgggaaaagcuggguugagagggcgaaaaaggaugaggu

SEQ ID NO: 17 - RNA, 22 nt, Homo sapiens
aaaagcuggguugagagggcga

SEQ ID NO: 18 - RNA, 79 nt, Homo sapiens
aauuaaucccucucuuucuaguucuuccuagagugaggaaaagcuggguugagagggcaaacaaauuaacuaauuaauu

SEQ ID NO: 19 - RNA, 137 nt, Homo sapiens
uguuauuuuuugucuucuaccuaagaauucugucucuuaggcuuucucuucccagauuucccaaaguugggaaaagcuggguugagagggcaaaaggaaaaaaaaagaauucugucucugacauaauuagauaggga

SEQ ID NO: 20 - RNA, 22 nt, Homo sapiens
aaaagcuggguugagagggcaa

SEQ ID NO: 21 - RNA, 50 nt, Homo sapiens
cuucucuuuccaguucuucccagaauugggaaaagcuggguugagagggu

SEQ ID NO: 22 - RNA, 20 nt, Homo sapiens
aaaagcuggguugagagggu

SEQ ID NO: 23 - RNA, 53 nt, Homo sapiens
gccuucucuucccaguucuuccuggagucggggaaaagcuggguugagaaggu

SEQ ID NO: 24 - RNA, 18 nt, Homo sapiens
aaagcuggguugagaagg

SEQ ID NO: 25 - RNA, 77 nt, Homo sapiens
guggccucguucaaguaauccaggauaggcugugcaggucccaaugggccuauucuugguuacuugcacggggacgc

SEQ ID NO: 26 - RNA, 84 nt, Homo sapiens
ggcuguggcuggauucaaguaauccaggauaggcuguuuccaucugugaggccuauucuugauuacuuguuucuggaggcagcu

SEQ ID NO: 27 - RNA, 22 nt, Homo sapiens
uucaaguaauccaggauaggcu

SEQ ID NO: 28 - RNA, 22 nt, Homo sapiens
ccuauucuugguuacuugcacg

SEQ ID NO: 29 - RNA, 22 nt, Homo sapiens
ccuauucuugauuacuuguuuc

SEQ ID NO: 30 - RNA, 62 nt, Homo sapiens
gguagggggcgggcuccggcgcugggaccccacuaggguggcgccuuggccccgccccgccc

SEQ ID NO: 31 - RNA, 18 nt, Homo sapiens
agggggcgggcuccggcg

SEQ ID NO: 32 - RNA, 100 nt, Homo sapiens
gugagcgggcgcggcagggaucgcgggcggguggcggccuagggcgcggagggcggaccgggaauggcgcgccgugcgccgccggcguaacugcggcgcu

SEQ ID NO: 33 - RNA, 25 nt, Homo sapiens
agggaucgcgggcggguggcggccu

SEQ ID NO: 34 - RNA, 64 nt, Homo sapiens
augacugauuucuuuugguguucagagucaauauaauuuucuagcaccaucugaaaucgguuau

SEQ ID NO: 35 - RNA, 22 nt, Homo sapiens
uagcaccaucugaaaucgguua

SEQ ID NO: 36 - RNA, 67 nt, Homo sapiens
ucuauuuagccaauuguccaucuuuagcuauucugaaugccuaaagauagacaauuggcuaaauaga

SEQ ID NO: 37 - RNA, 22 nt, Homo sapiens
uuagccaauuguccaucuuuag

SEQ ID NO: 38 - RNA, 22 nt, Homo sapiens
aaagauagacaauuggcuaaau

SEQ ID NO: 39 - RNA, 97 nt, Homo sapiens
cauuggcaucuauuagguuggugcaaaaguaauugcgguuuuugccauuacuuucaguagcaaaaaucucaauuacuuuugcaccaacuuaauacuu

SEQ ID NO: 40 - RNA, 88 nt, Homo sapiens
guauuagguuggugcaaagguaauugcaguuuuucccauuuaaaauauggaaaaaaaaaucacaauuacuuuugcaucaaccuaauaa

SEQ ID NO: 41 - RNA, 84 nt, Homo sapiens
auuagguuggugcaaaaguaaucacaguuuuugacauuacuuucaaagacaaaaacuguaauuacuuuuggaccaaccuaauag

SEQ ID NO: 42 - RNA, 97 nt, Homo sapiens
cagacuauauauuuagguuggcgcaaaaguaauugugguuuuggccuuuauuuucaauggcaagaaccucaguugcuuuugugccaaccuaauacuu

SEQ ID NO: 43 - RNA, 89 nt, Homo sapiens
aguuauuagauuagugcaaaaguaauugcaguuuuugcauuacguucuauggcaaaacuguaauuacuuuuguaccaacauaauacuuc

SEQ ID NO: 44 - RNA, 97 nt, Homo sapiens
ggucggugcaaaaguaauugcuguuuuugccauuaaaaauaauggcauuaaaaguaauggcaaaaacggcaaugacuuuuguaccaaucuaauaucu

SEQ ID NO: 45 - RNA, 22 nt, Homo sapiens
caaaaaucucaauuacuuuugc

SEQ ID NO: 46 - RNA, 22 nt, Homo sapiens
aaagguaauugcaguuuuuccc

SEQ ID NO: 47 - RNA, 22 nt, Homo sapiens
ugcaaaaguaaucacaguuuuu

SEQ ID NO: 48 - RNA, 19 nt, Homo sapiens
aaaaacuguaauuacuuuu

SEQ ID NO: 49 - RNA, 22 nt, Homo sapiens
caagaaccucaguugcuuuugu

SEQ ID NO: 50 - RNA, 23 nt, Homo sapiens
ugcaaaaguaauugcaguuuuug

SEQ ID NO: 51 - RNA, 22 nt, Homo sapiens
aaaacuguaauuacuuuuguac

SEQ ID NO: 52 - RNA, 22 nt, Homo sapiens
aacggcaaugacuuuuguacca

SEQ ID NO: 53 - RNA, 110 nt, Homo sapiens
ccagagguuguaacguugucuauauauacccuguagaaccgaauuugugugguauccguauagucacagauucgauucuaggggaauauauggucgaugcaaaaacuuca

SEQ ID NO: 54 - RNA, 23 nt, Homo sapiens
uacccuguagaaccgaauuugug

SEQ ID NO: 55 - RNA, 22 nt, Homo sapiens
acagauucgauucuaggggaau

SEQ ID NO: 56 - RNA, 85 nt, Homo sapiens
gugagcagucuccaccaccuccccugcaaacguccaguggugcagagguaauggacguuggcucugguggugauggacaguccga

SEQ ID NO: 57 - RNA, 22 nt, Homo sapiens
ccaccuccccugcaaacgucca

SEQ ID NO: 58 - RNA, 18 nt, Homo sapiens
acguuggcucugguggug

SEQ ID NO: 59 - RNA, 82 nt, Homo sapiens
ugagcccucggaggacuccauuuguuuugaugauggauucuuaugcuccaucaucgucucaaaugagucuucagaggguucu

SEQ ID NO: 60 - RNA, 23 nt, Homo sapiens
acuccauuuguuuugaugaugga

SEQ ID NO: 61 - RNA, 22 nt, Homo sapiens
caucaucgucucaaaugagucu

SEQ ID NO: 62 - RNA, 110 nt, Homo sapiens
ccgcagagugugacuccuguucuguguauggcacugguagaauucacugugaacagucucagucagugaauuaccgaagggccauaaacagagcagagacagauccacga

SEQ ID NO: 63 - RNA, 22 nt, Homo sapiens
uauggcacugguagaauucacu

SEQ ID NO: 64 - RNA, 22 nt, Homo sapiens
gugaauuaccgaagggccauaa

SEQ ID NO: 65 - RNA, 80 nt, Homo sapiens
aggacccuuccagagggcccccccucaauccuguugugccuaauucagaggguuggguggaggcucuccugaagggcucu

SEQ ID NO: 66 - RNA, 21 nt, Homo sapiens
agggcccccccucaauccugu

SEQ ID NO: 67 - RNA, 89 nt, Homo sapiens
accaugcuguaguguguguaaacauccuacacucucagcugugagcucaagguggcugggagaggguuguuuacuccuucugccaugga

SEQ ID NO: 68 - RNA, 72 nt, Homo sapiens
agauacuguaaacauccuacacucucagcuguggaaaguaagaaagcugggagaaggcuguuuacucuuucu

SEQ ID NO: 69 - RNA, 23 nt, Homo sapiens
uguaaacauccuacacucucagc

SEQ ID NO: 70 - RNA, 22 nt, Homo sapiens
cugggagaggguuguuuacucc

SEQ ID NO: 71 - RNA, 22 nt, Homo sapiens
cugggagaaggcuguuuacucu

SEQ ID NO: 72 - RNA, 91 nt, Homo sapiens
acgccccccgccccgccaccgccuuggaggcugaccucuuacuuucggucggucuucuucccugggcuugguuugggggcgggggaguguc

SEQ ID NO: 73 - RNA, 17 nt, Homo sapiens
ccccgccaccgccuugg

SEQ ID NO: 74 - RNA, 88 nt, Homo sapiens
ucccaugcugugacccuccaaagggaagcgcuuucuguuuguuuucucuuaaacaaagugccucccuuuagaguguuaccguuuggga

SEQ ID NO: 75 - RNA, 25 nt, Homo sapiens
ccuccaaagggaagcgcuuucuguu

SEQ ID NO: 76 - RNA, 22 nt, Homo sapiens
caaagugccucccuuuagagug

SEQ ID NO: 77 - RNA, 79 nt, Homo sapiens
gugcuuaaagaauggcuguccguaguauggucucuauauuuaugaugauuaauaucggacaaccauuguuuuaguaucc

SEQ ID NO: 78 - RNA, 22 nt, Homo sapiens
aauggcuguccguaguaugguc

SEQ ID NO: 79 - RNA, 21 nt, Homo sapiens
uuaauaucggacaaccauugu

SEQ ID NO: 80 - RNA, 68 nt, Homo sapiens
gcauccuguacugagcugccccgaggcccuucaugcugcccagcucggggcagcucaguacaggauac

SEQ ID NO: 81 - RNA, 22 nt, Homo sapiens
uccuguacugagcugccccgag

SEQ ID NO: 82 - RNA, 21 nt, Homo sapiens
cggggcagcucaguacaggau

SEQ ID NO: 83 - RNA, 85 nt, Homo sapiens
gugaggacucgggagguggaggguggugccgccggggccgggcgcuguuucagcucgcuucuccccccaccuccucucuccucag

SEQ ID NO: 84 - RNA, 21 nt, Homo sapiens
ccccaccuccucucuccucag

SEQ ID NO: 85 - RNA, 75 nt, Homo sapiens
gugagggcaugcaggccuggauggggcagcugggaugguccaaaaggguggccucaccagcccuguguucccuag

SEQ ID NO: 86 - RNA, 26 nt, Homo sapiens
gugagggcaugcaggccuggaugggg

SEQ ID NO: 87 - RNA, 22 nt, Homo sapiens
ucaccagcccuguguucccuag

SEQ ID NO: 88 - RNA, 88 nt, Homo sapiens
aucucuuacacaggcugaccgauuucuccugguguucagagucuguuuuugucuagcaccauuugaaaucgguuaugauguaggggga

SEQ ID NO: 89 - RNA, 22 nt, Homo sapiens
ugaccgauuucuccugguguuc

SEQ ID NO: 90 - RNA, 22 nt, Homo sapiens
uagcaccauuugaaaucgguua

SEQ ID NO: 91 - RNA, 77 nt, Homo sapiens
aaaugguuauguccuuugccuauucuauuuaagacacccuguaccuuaaauagaguaggcaaaggacagaaacauuu

SEQ ID NO: 92 - RNA, 22 nt, Homo sapiens
uccuuugccuauucuauuuaag

SEQ ID NO: 93 - RNA, 22 nt, Homo sapiens
uaaauagaguaggcaaaggaca

SEQ ID NO: 94 - RNA, 73 nt, Homo sapiens
ggcgaggggaggcgcaggcucggaaaggcgcgcgaggcuccaggcuccuucccgauccaccgcucuccucgcu

SEQ ID NO: 95 - RNA, 23 nt, Homo sapiens
ggaggcgcaggcucggaaaggcg

SEQ ID NO: 96 - RNA, 70 nt, Homo sapiens
ggagauauugcacauuacuaaguugcauguugucacggccucaaugcaauuuagugugugugauauuuuc

SEQ ID NO: 97 - RNA, 22 nt, Homo sapiens
uauugcacauuacuaaguugca

SEQ ID NO: 98 - RNA, 22 nt, Homo sapiens
caauuuagugugugugauauuu

SEQ ID NO: 99 - RNA, 75 nt, Homo sapiens
uguugucggguggaucacgaugcaauuuugaugaguaucauaggagaaaaauugcacgguauccaucuguaaacc

SEQ ID NO: 100 - RNA, 22 nt, Homo sapiens
cggguggaucacgaugcaauuu

SEQ ID NO: 101 - RNA, 22 nt, Homo sapiens
aauugcacgguauccaucugua

SEQ ID NO: 102 - RNA, 98 nt, Homo sapiens
uggccgacggggcgcgcgcggccuggaggggcggggcggacgcagagccgcguuuagucuaucgcugcgguugcgagcgcuguagggagccugugcug

SEQ ID NO: 103 - RNA, 23 nt, Homo sapiens
aucgcugcgguugcgagcgcugu

SEQ ID NO: 104 - RNA, 89 nt, Homo sapiens
ggagaggcugugcuguggggcaggcgcaggccugagcccugguuucgggcugccugggucucuggccugcgcgugacuuugggguggcu

SEQ ID NO: 105 - RNA, 24 nt, Homo sapiens
ugccugggucucuggccugcgcgu

SEQ ID NO: 106 - RNA, 88 nt, Homo sapiens
aacugcccucaaggagcuuacaaucuagcuggggguaaaugacuugcacaugaacacaacuagacugugagcuucuagagggcaggga

SEQ ID NO: 107 - RNA, 23 nt, Homo sapiens
aaggagcuuacaaucuagcuggg

SEQ ID NO: 108 - RNA, 22 nt, Homo sapiens
caacuagacugugagcuucuag

SEQ ID NO: 109 - RNA, 103 nt, Homo sapiens
ugaggcaggagaauugcuugaacccgggugguggagguugcagugagccaagauugcgccacugcacuccagccugggcgacaaagcaagacucuuucuugga

SEQ ID NO: 110 - RNA, 25 nt, Homo sapiens
gggcgacaaagcaagacucuuucuu

SEQ ID NO: 111 - RNA, 51 nt, Homo sapiens
ucuguguaaaccuggcaauuuucacuuaauugcuuccauguuuauaaaaga

SEQ ID NO: 112 - RNA, 17 nt, Homo sapiens
uaauugcuuccauguuu

SEQ ID NO: 113 - RNA, 77 nt, Homo sapiens
ccucgagagcugauaagaacagaggcccagauugaaguugaauagugcugggccuuuguuuuuaccaaguucccugg

SEQ ID NO: 114 - RNA, 23 nt, Homo sapiens
cugauaagaacagaggcccagau

SEQ ID NO: 115 - RNA, 76 nt, Homo sapiens
gagggggaagacgggaggaaagaagggagugguuccaucacgccuccucacuccucuccucccgucuucuccucuc

SEQ ID NO: 116 - RNA, 21 nt, Homo sapiens
ucacuccucuccucccgucuu

SEQ ID NO: 117 - RNA, 97 nt, Homo sapiens
ugaugcuuugcuggcuggugcagugccugagggaguaagagcccuguuguuguaagauagugucuuacucccucaggcacaucuccaacaagucucu

SEQ ID NO: 118 - RNA, 97 nt, Homo sapiens
ugaugcuuugcuggcuggugcagugccugagggaguaagagcccuguuguugucagauagugucuuacucccucaggcacaucuccagcgagucucu

SEQ ID NO: 119 - RNA, 95 nt, Homo sapiens
gaugcuuugcuggcuggugcagugccugagggaguaagaguccuguuguuguaagauagugucuuacucccucaggcacaucuccaacaagucuc

SEQ ID NO: 120 - RNA, 22 nt, Homo sapiens
ugucuuacucccucaggcacau

SEQ ID NO: 121 - RNA, 99 nt, Homo sapiens
uuuagcgguuucucccugaagugauguguaacugaucaggaucuacucaugucgucuuugguaaaguuaugucgcuugucagggugaggagaguuuuug

SEQ ID NO: 122 - RNA, 24 nt, Homo sapiens
cugaagugauguguaacugaucag

SEQ ID NO: 123 - RNA, 96 nt, Homo sapiens
ugggggagugaagaguagauaaaauauugguaccugaugaaucugaggccagguuucaauacuuuaucugcucuucauuuccccauaucuacuuac

SEQ ID NO: 124 - RNA, 21 nt, Homo sapiens
uagauaaaauauugguaccug